SEQUENCE LISTING (522 sequences)

SEQ ID NO: 1 - RNA, 22 nt, Artificial Sequence
  Artificially synthesized
gcggauguagccaaguggauca

SEQ ID NO: 2 - RNA, 19 nt, Artificial Sequence
  Artificially synthesized
gcggauguagccaagugga

SEQ ID NO: 3 - RNA, 22 nt, Artificial Sequence
  Artificially synthesized
ucaauucccgucguucgcccca

SEQ ID NO: 4 - RNA, 19 nt, Artificial Sequence
  Artificially synthesized
auucccgucguucgcccca

SEQ ID NO: 5 - RNA, 22 nt, Artificial Sequence
  Artificially synthesized
gggauuguaguucaaucgguca

SEQ ID NO: 6 - RNA, 19 nt, Artificial Sequence
  Artificially synthesized
gggauuguaguucaaucgg

SEQ ID NO: 7 - RNA, 22 nt, Artificial Sequence
  Artificially synthesized
ucgagccccgucagucccgcca

SEQ ID NO: 8 - RNA, 19 nt, Artificial Sequence
  Artificially synthesized
agccccgucagucccgcca

SEQ ID NO: 9 - RNA, 22 nt, Artificial Sequence
  Artificially synthesized
gcggauauagucgaaugguaaa

SEQ ID NO: 10 - RNA, 19 nt, Artificial Sequence
  Artificially synthesized
gcggauauagucgaauggu

SEQ ID NO: 11 - RNA, 22 nt, Artificial Sequence
  Artificially synthesized
ucgauucccgcuauccgcccca

SEQ ID NO: 12 - RNA, 19 nt, Artificial Sequence
  Artificially synthesized
auucccgcuauccgcccca

SEQ ID NO: 13 - RNA, 22 nt, Artificial Sequence
  Artificially synthesized
gccuugguggugaaaugguaga

SEQ ID NO: 14 - RNA, 19 nt, Artificial Sequence
  Artificially synthesized
gccuugguggugaaauggu

SEQ ID NO: 15 - RNA, 22 nt, Artificial Sequence
  Artificially synthesized
ucgaguccucuucaaggcacca

SEQ ID NO: 16 - RNA, 19 nt, Artificial Sequence
  Artificially synthesized
aguccucuucaaggcacca

SEQ ID NO: 17 - RNA, 22 nt, Artificial Sequence
  Artificially synthesized
cgcggaguagagcaguuuggua

SEQ ID NO: 18 - RNA, 19 nt, Artificial Sequence
  Artificially synthesized
cgcggaguagagcaguuug

SEQ ID NO: 19 - RNA, 22 nt, Artificial Sequence
  Artificially synthesized
ucaaauccugucuccgcaacca

SEQ ID NO: 20 - RNA, 19 nt, Artificial Sequence
  Artificially synthesized
aauccugucuccgcaacca

SEQ ID NO: 21 - RNA, 22 nt, Artificial Sequence
  Artificially synthesized
ggagagauggcugaguggacua

SEQ ID NO: 22 - RNA, 19 nt, Artificial Sequence
  Artificially synthesized
ggagagauggcugagugga

SEQ ID NO: 23 - RNA, 22 nt, Artificial Sequence
  Artificially synthesized
ggagagauggcugaguggacua

SEQ ID NO: 24 - RNA, 19 nt, Artificial Sequence
  Artificially synthesized
aaucccucucuuuccgcca

SEQ ID NO: 25 - RNA, 22 nt, Artificial Sequence
  Artificially synthesized
uggggcguggccaagugguaag

SEQ ID NO: 26 - RNA, 19 nt, Artificial Sequence
  Artificially synthesized
uggggcguggccaaguggu

SEQ ID NO: 27 - RNA, 22 nt, Artificial Sequence
  Artificially synthesized
ucgaauccuuccgucccagcca

SEQ ID NO: 28 - RNA, 19 nt, Artificial Sequence
  Artificially synthesized
aauccuuccgucccagcca

SEQ ID NO: 29 - RNA, 22 nt, Artificial Sequence
  Artificially synthesized
gcccccaucgucuagugguuca

SEQ ID NO: 30 - RNA, 19 nt, Artificial Sequence
  Artificially synthesized
gcccccaucgucuaguggu

SEQ ID NO: 31 - RNA, 22 nt, Artificial Sequence
  Artificially synthesized
ucgacuuccccuggggguacca

SEQ ID NO: 32 - RNA, 19 nt, Artificial Sequence
  Artificially synthesized
acuuccccuggggguacca

SEQ ID NO: 33 - RNA, 22 nt, Artificial Sequence
  Artificially synthesized
uccucaguagcucagugguaga

SEQ ID NO: 34 - RNA, 19 nt, Artificial Sequence
  Artificially synthesized
uccucaguagcucaguggu

SEQ ID NO: 35 - RNA, 22 nt, Artificial Sequence
  Artificially synthesized
ucgaauccuaccuggggagcca

SEQ ID NO: 36 - RNA, 19 nt, Artificial Sequence
  Artificially synthesized
aauccuaccuggggagcca

SEQ ID NO: 37 - RNA, 22 nt, Artificial Sequence
  Artificially synthesized
agggauguagcgcagcuuggua

SEQ ID NO: 38 - RNA, 19 nt, Artificial Sequence
  Artificially synthesized
agggauguagcgcagcuug

SEQ ID NO: 39 - RNA, 22 nt, Artificial Sequence
  Artificially synthesized
gguucaaauccugucaucccua

SEQ ID NO: 40 - RNA, 19 nt, Artificial Sequence
  Artificially synthesized
ucaaauccugucaucccua

SEQ ID NO: 41 - RNA, 22 nt, Artificial Sequence
  Artificially synthesized
gguuccauggucuaguggucag

SEQ ID NO: 42 - RNA, 19 nt, Artificial Sequence
  Artificially synthesized
gguuccauggucuaguggu

SEQ ID NO: 43 - RNA, 22 nt, Artificial Sequence
  Artificially synthesized
ucaggucucgguggaaccucca

SEQ ID NO: 44 - RNA, 19 nt, Artificial Sequence
  Artificially synthesized
ggucucgguggaaccucca

SEQ ID NO: 45 - RNA, 22 nt, Artificial Sequence
  Artificially synthesized
uccguugucguccagcgguuag

SEQ ID NO: 46 - RNA, 19 nt, Artificial Sequence
  Artificially synthesized
uccguugucguccagcggu

SEQ ID NO: 47 - RNA, 22 nt, Artificial Sequence
  Artificially synthesized
ucguuucccggcaacggaacca

SEQ ID NO: 48 - RNA, 19 nt, Artificial Sequence
  Artificially synthesized
uuucccggcaacggaacca

SEQ ID NO: 49 - RNA, 22 nt, Artificial Sequence
  Artificially synthesized
ggcuagguaacauaauggaaau

SEQ ID NO: 50 - RNA, 19 nt, Artificial Sequence
  Artificially synthesized
ggcuagguaacauaaugga

SEQ ID NO: 51 - RNA, 22 nt, Artificial Sequence
  Artificially synthesized
ucgaccccguccuuggccucca

SEQ ID NO: 52 - RNA, 19 nt, Artificial Sequence
  Artificially synthesized
accccguccuuggccucca

SEQ ID NO: 53 - RNA, 22 nt, Artificial Sequence
  Artificially synthesized
agcgggguagaguaauggucaa

SEQ ID NO: 54 - RNA, 19 nt, Artificial Sequence
  Artificially synthesized
agcgggguagaguaauggu

SEQ ID NO: 55 - RNA, 22 nt, Artificial Sequence
  Artificially synthesized
ucgaauccuguccccgccucca

SEQ ID NO: 56 - RNA, 19 nt, Artificial Sequence
  Artificially synthesized
aauccuguccccgccucca

SEQ ID NO: 57 - RNA, 22 nt, Artificial Sequence
  Artificially synthesized
cgagguguagcgcagucugguc

SEQ ID NO: 58 - RNA, 19 nt, Artificial Sequence
  Artificially synthesized
cgagguguagcgcagucug

SEQ ID NO: 59 - RNA, 22 nt, Artificial Sequence
  Artificially synthesized
ucgaauccugucaccuugacca

SEQ ID NO: 60 - RNA, 19 nt, Artificial Sequence
  Artificially synthesized
aauccugucaccuugacca

SEQ ID NO: 61 - RNA, 22 nt, Artificial Sequence
  Artificially synthesized
gcaccaguggucuagugguaga

SEQ ID NO: 62 - RNA, 19 nt, Artificial Sequence
  Artificially synthesized
gcaccaguggucuaguggu

SEQ ID NO: 63 - RNA, 22 nt, Artificial Sequence
  Artificially synthesized
ucguuucccggcuggugcacca

SEQ ID NO: 64 - RNA, 19 nt, Artificial Sequence
  Artificially synthesized
uuucccggcuggugcacca

SEQ ID NO: 65 - RNA, 22 nt, Artificial Sequence
  Artificially synthesized
gucguuguaguauagugguaag

SEQ ID NO: 66 - RNA, 19 nt, Artificial Sequence
  Artificially synthesized
gucguuguaguauaguggu

SEQ ID NO: 67 - RNA, 22 nt, Artificial Sequence
  Artificially synthesized
ucgauccccggcaacggcgcca

SEQ ID NO: 68 - RNA, 19 nt, Artificial Sequence
  Artificially synthesized
auccccggcaacggcgcca

SEQ ID NO: 69 - RNA, 22 nt, Artificial Sequence
  Artificially synthesized
ccgaccuuagcucaguugguag

SEQ ID NO: 70 - RNA, 19 nt, Artificial Sequence
  Artificially synthesized
ccgaccuuagcucaguugg

SEQ ID NO: 71 - RNA, 22 nt, Artificial Sequence
  Artificially synthesized
ucgaauccggcuggucggacca

SEQ ID NO: 72 - RNA, 19 nt, Artificial Sequence
  Artificially synthesized
aauccggcuggucggacca

SEQ ID NO: 73 - RNA, 22 nt, Artificial Sequence
  Artificially synthesized
ggggauguagcucagaugguag

SEQ ID NO: 74 - RNA, 19 nt, Artificial Sequence
  Artificially synthesized
ggggauguagcucagaugg

SEQ ID NO: 75 - RNA, 22 nt, Artificial Sequence
  Artificially synthesized
ucgauaccccgcaucuccacca

SEQ ID NO: 76 - RNA, 19 nt, Artificial Sequence
  Artificially synthesized
auaccccgcaucuccacca

SEQ ID NO: 77 - RNA, 22 nt, Artificial Sequence
  Artificially synthesized
uccguuguagucuaguugguca

SEQ ID NO: 78 - RNA, 19 nt, Artificial Sequence
  Artificially synthesized
uccguuguagucuaguugg

SEQ ID NO: 79 - RNA, 22 nt, Artificial Sequence
  Artificially synthesized
ucaagucccggcaacggaacca

SEQ ID NO: 80 - RNA, 19 nt, Artificial Sequence
  Artificially synthesized
agucccggcaacggaacca

SEQ ID NO: 81 - RNA, 22 nt, Artificial Sequence
  Artificially synthesized
gucccuuucguccagugguuag

SEQ ID NO: 82 - RNA, 19 nt, Artificial Sequence
  Artificially synthesized
gucccuuucguccaguggu

SEQ ID NO: 83 - RNA, 22 nt, Artificial Sequence
  Artificially synthesized
ucgauucccguaagggguacca

SEQ ID NO: 84 - RNA, 19 nt, Artificial Sequence
  Artificially synthesized
auucccguaagggguacca

SEQ ID NO: 85 - RNA, 22 nt, Artificial Sequence
  Artificially synthesized
gcgccuguagcucaguggauag

SEQ ID NO: 86 - RNA, 19 nt, Artificial Sequence
  Artificially synthesized
gcgccuguagcucagugga

SEQ ID NO: 87 - RNA, 22 nt, Artificial Sequence
  Artificially synthesized
ucgaccccuaccuggcgcgcca

SEQ ID NO: 88 - RNA, 19 nt, Artificial Sequence
  Artificially synthesized
accccuaccuggcgcgcca

SEQ ID NO: 89 - RNA, 22 nt, Artificial Sequence
  Artificially synthesized
gguuucgugguguaguugguua

SEQ ID NO: 90 - RNA, 19 nt, Artificial Sequence
  Artificially synthesized
gguuucgugguguaguugg

SEQ ID NO: 91 - RNA, 22 nt, Artificial Sequence
  Artificially synthesized
ucgaacccgggcgaagccacca

SEQ ID NO: 92 - RNA, 19 nt, Artificial Sequence
  Artificially synthesized
aacccgggcgaagccacca

SEQ ID NO: 93 - RNA, 22 nt, Artificial Sequence
  Artificially synthesized
gucugggugguguagucgguua

SEQ ID NO: 94 - RNA, 19 nt, Artificial Sequence
  Artificially synthesized
gucugggugguguagucgg

SEQ ID NO: 95 - RNA, 22 nt, Artificial Sequence
  Artificially synthesized
ucgaacccgggcucagacacca

SEQ ID NO: 96 - RNA, 19 nt, Artificial Sequence
  Artificially synthesized
aacccgggcucagacacca

SEQ ID NO: 97 - RNA, 22 nt, Artificial Sequence
  Artificially synthesized
ggauggaugucugagcgguugg

SEQ ID NO: 98 - RNA, 19 nt, Artificial Sequence
  Artificially synthesized
ggauggaugucugagcggu

SEQ ID NO: 99 - RNA, 22 nt, Artificial Sequence
  Artificially synthesized
ucgaaucccucuccauccgcca

SEQ ID NO: 100 - RNA, 19 nt, Artificial Sequence
  Artificially synthesized
aaucccucuccauccgcca

SEQ ID NO: 101 - RNA, 22 nt, Artificial Sequence
  Artificially synthesized
gcggggauagcucaguugggag

SEQ ID NO: 102 - RNA, 19 nt, Artificial Sequence
  Artificially synthesized
gcggggauagcucaguugg

SEQ ID NO: 103 - RNA, 22 nt, Artificial Sequence
  Artificially synthesized
uugauccacguucaccgcacca

SEQ ID NO: 104 - RNA, 19 nt, Artificial Sequence
  Artificially synthesized
auccacguucaccgcacca

SEQ ID NO: 105 - RNA, 22 nt, Artificial Sequence
  Artificially synthesized
gcauccauggcugaaugguuaa

SEQ ID NO: 106 - RNA, 19 nt, Artificial Sequence
  Artificially synthesized
gcauccauggcugaauggu

SEQ ID NO: 107 - RNA, 22 nt, Artificial Sequence
  Artificially synthesized
ucaauuccuacuggaugcacca

SEQ ID NO: 108 - RNA, 19 nt, Artificial Sequence
  Artificially synthesized
auuccuacuggaugcacca

SEQ ID NO: 109 - RNA, 22 nt, Artificial Sequence
  Artificially synthesized
ggguugcuaacucaacgguaga

SEQ ID NO: 110 - RNA, 19 nt, Artificial Sequence
  Artificially synthesized
ggguugcuaacucaacggu

SEQ ID NO: 111 - RNA, 22 nt, Artificial Sequence
  Artificially synthesized
ucgaaucccgggcaacccacca

SEQ ID NO: 112 - RNA, 19 nt, Artificial Sequence
  Artificially synthesized
aaucccgggcaacccacca

SEQ ID NO: 113 - RNA, 22 nt, Artificial Sequence
  Artificially synthesized
ggagagauggcugagugguuga

SEQ ID NO: 114 - RNA, 19 nt, Artificial Sequence
  Artificially synthesized
ggagagauggcugaguggu

SEQ ID NO: 115 - RNA, 22 nt, Artificial Sequence
  Artificially synthesized
ucgaaucccucucucuccucca

SEQ ID NO: 116 - RNA, 19 nt, Artificial Sequence
  Artificially synthesized
aaucccucucucuccucca

SEQ ID NO: 117 - RNA, 22 nt, Artificial Sequence
  Artificially synthesized
aggagagauggccgagugguug

SEQ ID NO: 118 - RNA, 19 nt, Artificial Sequence
  Artificially synthesized
aggagagauggccgagugg

SEQ ID NO: 119 - RNA, 22 nt, Artificial Sequence
  Artificially synthesized
ucgaaucccucucuuuccgcca

SEQ ID NO: 120 - RNA, 19 nt, Artificial Sequence
  Artificially synthesized
aaucccucucuuuccgcca

SEQ ID NO: 121 - RNA, 22 nt, Artificial Sequence
  Artificially synthesized
gcggguauaguuuagugguaaa

SEQ ID NO: 122 - RNA, 19 nt, Artificial Sequence
  Artificially synthesized
gcggguauaguuuaguggu

SEQ ID NO: 123 - RNA, 22 nt, Artificial Sequence
  Artificially synthesized
ucgauucccgcuacccgcucca

SEQ ID NO: 124 - RNA, 19 nt, Artificial Sequence
  Artificially synthesized
auucccgcuacccgcucca

SEQ ID NO: 125 - RNA, 22 nt, Artificial Sequence
  Artificially synthesized
gcguccauugucuaauggauag

SEQ ID NO: 126 - RNA, 19 nt, Artificial Sequence
  Artificially synthesized
gcguccauugucuaaugga

SEQ ID NO: 127 - RNA, 22 nt, Artificial Sequence
  Artificially synthesized
ucaaauccuauuggacgcacca

SEQ ID NO: 128 - RNA, 19 nt, Artificial Sequence
  Artificially synthesized
aauccuauuggacgcacca

SEQ ID NO: 129 - RNA, 22 nt, Artificial Sequence
  Artificially synthesized
gggccuguagcucagaggauua

SEQ ID NO: 130 - RNA, 19 nt, Artificial Sequence
  Artificially synthesized
gggccuguagcucagagga

SEQ ID NO: 131 - RNA, 22 nt, Artificial Sequence
  Artificially synthesized
ucgaaucccuccucgcccacca

SEQ ID NO: 132 - RNA, 19 nt, Artificial Sequence
  Artificially synthesized
aaucccuccucgcccacca

SEQ ID NO: 133 - RNA, 22 nt, Artificial Sequence
  Artificially synthesized
ggcgauauggccgagugguaag

SEQ ID NO: 134 - RNA, 19 nt, Artificial Sequence
  Artificially synthesized
ggcgauauggccgaguggu

SEQ ID NO: 135 - RNA, 22 nt, Artificial Sequence
  Artificially synthesized
ucaaauccgggugucgccucca

SEQ ID NO: 136 - RNA, 19 nt, Artificial Sequence
  Artificially synthesized
aauccgggugucgccucca

SEQ ID NO: 137 - RNA, 22 nt, Artificial Sequence
  Artificially synthesized
gggucgaugcccgagcgguuaa

SEQ ID NO: 138 - RNA, 19 nt, Artificial Sequence
  Artificially synthesized
gggucgaugcccgagcggu

SEQ ID NO: 139 - RNA, 22 nt, Artificial Sequence
  Artificially synthesized
ucaaauccagcucggcccacca

SEQ ID NO: 140 - RNA, 19 nt, Artificial Sequence
  Artificially synthesized
aauccagcucggcccacca

SEQ ID NO: 141 - RNA, 22 nt, Artificial Sequence
  Artificially synthesized
gcccuuuuaacucagcgguaga

SEQ ID NO: 142 - RNA, 19 nt, Artificial Sequence
  Artificially synthesized
gcccuuuuaacucagcggu

SEQ ID NO: 143 - RNA, 22 nt, Artificial Sequence
  Artificially synthesized
ucaaauccgauaaggggcucca

SEQ ID NO: 144 - RNA, 19 nt, Artificial Sequence
  Artificially synthesized
aauccgauaaggggcucca

SEQ ID NO: 145 - RNA, 22 nt, Artificial Sequence
  Artificially synthesized
gccugcuuagcucagagguuag

SEQ ID NO: 146 - RNA, 19 nt, Artificial Sequence
  Artificially synthesized
gccugcuuagcucagaggu

SEQ ID NO: 147 - RNA, 22 nt, Artificial Sequence
  Artificially synthesized
ucgauuccgauagccggcucca

SEQ ID NO: 148 - RNA, 19 nt, Artificial Sequence
  Artificially synthesized
auuccgauagccggcucca

SEQ ID NO: 149 - RNA, 22 nt, Artificial Sequence
  Artificially synthesized
accuacuuaacucagugguuag

SEQ ID NO: 150 - RNA, 19 nt, Artificial Sequence
  Artificially synthesized
accuacuuaacucaguggu

SEQ ID NO: 151 - RNA, 22 nt, Artificial Sequence
  Artificially synthesized
ucaaauccaauaguagguacca

SEQ ID NO: 152 - RNA, 19 nt, Artificial Sequence
  Artificially synthesized
aauccaauaguagguacca

SEQ ID NO: 153 - RNA, 22 nt, Artificial Sequence
  Artificially synthesized
ggggauauggcggaauugguag

SEQ ID NO: 154 - RNA, 19 nt, Artificial Sequence
  Artificially synthesized
ggggauauggcggaauugg

SEQ ID NO: 155 - RNA, 22 nt, Artificial Sequence
  Artificially synthesized
ucaagucccucuauccccacca

SEQ ID NO: 156 - RNA, 19 nt, Artificial Sequence
  Artificially synthesized
agucccucuauccccacca

SEQ ID NO: 157 - RNA, 22 nt, Artificial Sequence
  Artificially synthesized
gccgcuauggugaaaucgguag

SEQ ID NO: 158 - RNA, 19 nt, Artificial Sequence
  Artificially synthesized
gccgcuauggugaaaucgg

SEQ ID NO: 159 - RNA, 22 nt, Artificial Sequence
  Artificially synthesized
ucgaguccgaguggcggcacca

SEQ ID NO: 160 - RNA, 19 nt, Artificial Sequence
  Artificially synthesized
aguccgaguggcggcacca

SEQ ID NO: 161 - RNA, 22 nt, Artificial Sequence
  Artificially synthesized
gucgggauagcucagcugguag

SEQ ID NO: 162 - RNA, 19 nt, Artificial Sequence
  Artificially synthesized
gucgggauagcucagcugg

SEQ ID NO: 163 - RNA, 22 nt, Artificial Sequence
  Artificially synthesized
ucaaaucugguuccuggcacca

SEQ ID NO: 164 - RNA, 19 nt, Artificial Sequence
  Artificially synthesized
aaucugguuccuggcacca

SEQ ID NO: 165 - RNA, 22 nt, Artificial Sequence
  Artificially synthesized
agggcuauagcucaguuaggua

SEQ ID NO: 166 - RNA, 19 nt, Artificial Sequence
  Artificially synthesized
agggcuauagcucaguuag

SEQ ID NO: 167 - RNA, 22 nt, Artificial Sequence
  Artificially synthesized
ccgaguccguauagcccuacca

SEQ ID NO: 168 - RNA, 19 nt, Artificial Sequence
  Artificially synthesized
aguccguauagcccuacca

SEQ ID NO: 169 - RNA, 22 nt, Artificial Sequence
  Artificially synthesized
agggauauaacucagcgguaga

SEQ ID NO: 170 - RNA, 19 nt, Artificial Sequence
  Artificially synthesized
agggauauaacucagcggu

SEQ ID NO: 171 - RNA, 22 nt, Artificial Sequence
  Artificially synthesized
ucgagccugauuaucccuacca

SEQ ID NO: 172 - RNA, 19 nt, Artificial Sequence
  Artificially synthesized
agccugauuaucccuacca

SEQ ID NO: 173 - RNA, 22 nt, Artificial Sequence
  Artificially synthesized
gcgcucuuaguucaguucggua

SEQ ID NO: 174 - RNA, 19 nt, Artificial Sequence
  Artificially synthesized
gcgcucuuaguucaguucg

SEQ ID NO: 175 - RNA, 22 nt, Artificial Sequence
  Artificially synthesized
ucaaauccuacagagcgugcca

SEQ ID NO: 176 - RNA, 19 nt, Artificial Sequence
  Artificially synthesized
aauccuacagagcgugcca

SEQ ID NO: 177 - RNA, 22 nt, Artificial Sequence
  Artificially synthesized
gggcuauuagcucagugguaga

SEQ ID NO: 178 - RNA, 19 nt, Artificial Sequence
  Artificially synthesized
gggcuauuagcucaguggu

SEQ ID NO: 179 - RNA, 22 nt, Artificial Sequence
  Artificially synthesized
ucaaguccaggauggcccacca

SEQ ID NO: 180 - RNA, 19 nt, Artificial Sequence
  Artificially synthesized
aguccaggauggcccacca

SEQ ID NO: 181 - RNA, 22 nt, Artificial Sequence
  Artificially synthesized
ggggauauagcucaguugguag

SEQ ID NO: 182 - RNA, 19 nt, Artificial Sequence
  Artificially synthesized
ggggauauagcucaguugg

SEQ ID NO: 183 - RNA, 22 nt, Artificial Sequence
  Artificially synthesized
ucgaguccgcuuaucuccacca

SEQ ID NO: 184 - RNA, 19 nt, Artificial Sequence
  Artificially synthesized
aguccgcuuaucuccacca

SEQ ID NO: 185 - RNA, 22 nt, Artificial Sequence
  Artificially synthesized
ggguguauagcucaguugguag

SEQ ID NO: 186 - RNA, 19 nt, Artificial Sequence
  Artificially synthesized
ggguguauagcucaguugg

SEQ ID NO: 187 - RNA, 22 nt, Artificial Sequence
  Artificially synthesized
ucaaguccugcuauacccacca

SEQ ID NO: 188 - RNA, 19 nt, Artificial Sequence
  Artificially synthesized
aguccugcuauacccacca

SEQ ID NO: 189 - RNA, 22 nt, Artificial Sequence
  Artificially synthesized
cacccuguagcucagaggaaga

SEQ ID NO: 190 - RNA, 19 nt, Artificial Sequence
  Artificially synthesized
cacccuguagcucagagga

SEQ ID NO: 191 - RNA, 22 nt, Artificial Sequence
  Artificially synthesized
ucaaguccuaccagguuaccca

SEQ ID NO: 192 - RNA, 19 nt, Artificial Sequence
  Artificially synthesized
aguccuaccagguuaccca

SEQ ID NO: 193 - RNA, 22 nt, Artificial Sequence
  Artificially synthesized
uggaguauagccaagugguaag

SEQ ID NO: 194 - RNA, 19 nt, Artificial Sequence
  Artificially synthesized
uggaguauagccaaguggu

SEQ ID NO: 195 - RNA, 22 nt, Artificial Sequence
  Artificially synthesized
ucgaauccuuuuacuccagcca

SEQ ID NO: 196 - RNA, 19 nt, Artificial Sequence
  Artificially synthesized
aauccuuuuacuccagcca

SEQ ID NO: 197 - RNA, 22 nt, Artificial Sequence
  Artificially synthesized
gggcuuauaguuuaauugguug

SEQ ID NO: 198 - RNA, 19 nt, Artificial Sequence
  Artificially synthesized
gggcuuauaguuuaauugg

SEQ ID NO: 199 - RNA, 22 nt, Artificial Sequence
  Artificially synthesized
ucgagcccuacuaagccuacca

SEQ ID NO: 200 - RNA, 19 nt, Artificial Sequence
  Artificially synthesized
agcccuacuaagccuacca

SEQ ID NO: 201 - RNA, 22 nt, Artificial Sequence
  Artificially synthesized
gcauccauggcugaaugguuaa

SEQ ID NO: 202 - RNA, 19 nt, Artificial Sequence
  Artificially synthesized
gcauccauggcugaauggu

SEQ ID NO: 203 - RNA, 22 nt, Artificial Sequence
  Artificially synthesized
ucaauuccuacuggaugcacca

SEQ ID NO: 204 - RNA, 19 nt, Artificial Sequence
  Artificially synthesized
auuccuacuggaugcacca

SEQ ID NO: 205 - RNA, 22 nt, Artificial Sequence
  Artificially synthesized
gggagaguggccgaguggucaa

SEQ ID NO: 206 - RNA, 19 nt, Artificial Sequence
  Artificially synthesized
gggagaguggccgaguggu

SEQ ID NO: 207 - RNA, 22 nt, Artificial Sequence
  Artificially synthesized
ucgaauccugccucucccacca

SEQ ID NO: 208 - RNA, 19 nt, Artificial Sequence
  Artificially synthesized
aauccugccucucccacca

SEQ ID NO: 209 - RNA, 22 nt, Artificial Sequence
  Artificially synthesized
ggagguauggcugaguggcuua

SEQ ID NO: 210 - RNA, 19 nt, Artificial Sequence
  Artificially synthesized
ggagguauggcugaguggc

SEQ ID NO: 211 - RNA, 22 nt, Artificial Sequence
  Artificially synthesized
ucgaaucccauuuccuccgcca

SEQ ID NO: 212 - RNA, 19 nt, Artificial Sequence
  Artificially synthesized
aaucccauuuccuccgcca

SEQ ID NO: 213 - RNA, 22 nt, Artificial Sequence
  Artificially synthesized
guucagguagcucagcugguua

SEQ ID NO: 214 - RNA, 19 nt, Artificial Sequence
  Artificially synthesized
guucagguagcucagcugg

SEQ ID NO: 215 - RNA, 22 nt, Artificial Sequence
  Artificially synthesized
ucgaauccacuucuaagcgcca

SEQ ID NO: 216 - RNA, 19 nt, Artificial Sequence
  Artificially synthesized
aauccacuucuaagcgcca

SEQ ID NO: 217 - RNA, 22 nt, Artificial Sequence
  Artificially synthesized
guaacgaucgaauaauggaagu

SEQ ID NO: 218 - RNA, 19 nt, Artificial Sequence
  Artificially synthesized
guaacgaucgaauaaugga

SEQ ID NO: 219 - RNA, 22 nt, Artificial Sequence
  Artificially synthesized
ucaaauccaauucguuacucca

SEQ ID NO: 220 - RNA, 19 nt, Artificial Sequence
  Artificially synthesized
aauccaauucguuacucca

SEQ ID NO: 221 - RNA, 22 nt, Artificial Sequence
  Artificially synthesized
agggauguagcgcagcuuggua

SEQ ID NO: 222 - RNA, 19 nt, Artificial Sequence
  Artificially synthesized
agggauguagcgcagcuug

SEQ ID NO: 223 - RNA, 22 nt, Artificial Sequence
  Artificially synthesized
uccaauccugucaucccuacca

SEQ ID NO: 224 - RNA, 19 nt, Artificial Sequence
  Artificially synthesized
aauccugucaucccuacca

SEQ ID NO: 225 - RNA, 22 nt, Artificial Sequence
  Artificially synthesized
gggcuauuagcucagugguaga

SEQ ID NO: 226 - RNA, 19 nt, Artificial Sequence
  Artificially synthesized
gggcuauuagcucaguggu

SEQ ID NO: 227 - RNA, 22 nt, Artificial Sequence
  Artificially synthesized
ucaaguccaggauggcccacca

SEQ ID NO: 228 - RNA, 19 nt, Artificial Sequence
  Artificially synthesized
aguccaggauggcccacca

SEQ ID NO: 229 - RNA, 22 nt, Artificial Sequence
  Artificially synthesized
gcggaaauagcuuaaugguaga

SEQ ID NO: 230 - RNA, 19 nt, Artificial Sequence
  Artificially synthesized
gcggaaauagcuuaauggu

SEQ ID NO: 231 - RNA, 22 nt, Artificial Sequence
  Artificially synthesized
ucaagucccuccuuccgcucca

SEQ ID NO: 232 - RNA, 19 nt, Artificial Sequence
  Artificially synthesized
agucccuccuuccgcucca

SEQ ID NO: 233 - RNA, 22 nt, Artificial Sequence
  Artificially synthesized
ugauccacuuggcuacauccgc

SEQ ID NO: 234 - RNA, 19 nt, Artificial Sequence
  Artificially synthesized
uccacuuggcuacauccgc

SEQ ID NO: 235 - RNA, 22 nt, Artificial Sequence
  Artificially synthesized
uggggcgaacgacgggaauuga

SEQ ID NO: 236 - RNA, 19 nt, Artificial Sequence
  Artificially synthesized
uggggcgaacgacgggaau

SEQ ID NO: 237 - RNA, 22 nt, Artificial Sequence
  Artificially synthesized
ugaccgauugaacuacaauccc

SEQ ID NO: 238 - RNA, 19 nt, Artificial Sequence
  Artificially synthesized
ccgauugaacuacaauccc

SEQ ID NO: 239 - RNA, 22 nt, Artificial Sequence
  Artificially synthesized
uggcgggacugacggggcucga

SEQ ID NO: 240 - RNA, 19 nt, Artificial Sequence
  Artificially synthesized
uggcgggacugacggggcu

SEQ ID NO: 241 - RNA, 22 nt, Artificial Sequence
  Artificially synthesized
uuuaccauucgacuauauccgc

SEQ ID NO: 242 - RNA, 19 nt, Artificial Sequence
  Artificially synthesized
accauucgacuauauccgc

SEQ ID NO: 243 - RNA, 22 nt, Artificial Sequence
  Artificially synthesized
uggggcggauagcgggaaucga

SEQ ID NO: 244 - RNA, 19 nt, Artificial Sequence
  Artificially synthesized
uggggcggauagcgggaau

SEQ ID NO: 245 - RNA, 22 nt, Artificial Sequence
  Artificially synthesized
ucuaccauuucaccaccaaggc

SEQ ID NO: 246 - RNA, 19 nt, Artificial Sequence
  Artificially synthesized
accauuucaccaccaaggc

SEQ ID NO: 247 - RNA, 22 nt, Artificial Sequence
  Artificially synthesized
uggugccuugaagaggacucga

SEQ ID NO: 248 - RNA, 19 nt, Artificial Sequence
  Artificially synthesized
uggugccuugaagaggacu

SEQ ID NO: 249 - RNA, 22 nt, Artificial Sequence
  Artificially synthesized
uaccaaacugcucuacuccgcg

SEQ ID NO: 250 - RNA, 19 nt, Artificial Sequence
  Artificially synthesized
caaacugcucuacuccgcg

SEQ ID NO: 251 - RNA, 22 nt, Artificial Sequence
  Artificially synthesized
ugguugcggagacaggauuuga

SEQ ID NO: 252 - RNA, 19 nt, Artificial Sequence
  Artificially synthesized
ugguugcggagacaggauu

SEQ ID NO: 253 - RNA, 22 nt, Artificial Sequence
  Artificially synthesized
uaguccacucagccaucucucc

SEQ ID NO: 254 - RNA, 19 nt, Artificial Sequence
  Artificially synthesized
uccacucagccaucucucc

SEQ ID NO: 255 - RNA, 22 nt, Artificial Sequence
  Artificially synthesized
uggcggaaagagagggauucga

SEQ ID NO: 256 - RNA, 19 nt, Artificial Sequence
  Artificially synthesized
uggcggaaagagagggauu

SEQ ID NO: 257 - RNA, 22 nt, Artificial Sequence
  Artificially synthesized
cuuaccacuuggccacgcccca

SEQ ID NO: 258 - RNA, 19 nt, Artificial Sequence
  Artificially synthesized
accacuuggccacgcccca

SEQ ID NO: 259 - RNA, 22 nt, Artificial Sequence
  Artificially synthesized
uggcugggacggaaggauucga

SEQ ID NO: 260 - RNA, 19 nt, Artificial Sequence
  Artificially synthesized
uggcugggacggaaggauu

SEQ ID NO: 261 - RNA, 22 nt, Artificial Sequence
  Artificially synthesized
ugaaccacuagacgaugggggc

SEQ ID NO: 262 - RNA, 19 nt, Artificial Sequence
  Artificially synthesized
accacuagacgaugggggc

SEQ ID NO: 263 - RNA, 22 nt, Artificial Sequence
  Artificially synthesized
ugguacccccaggggaagucga

SEQ ID NO: 264 - RNA, 19 nt, Artificial Sequence
  Artificially synthesized
ugguacccccaggggaagu

SEQ ID NO: 265 - RNA, 22 nt, Artificial Sequence
  Artificially synthesized
ucuaccacugagcuacugagga

SEQ ID NO: 266 - RNA, 19 nt, Artificial Sequence
  Artificially synthesized
accacugagcuacugagga

SEQ ID NO: 267 - RNA, 22 nt, Artificial Sequence
  Artificially synthesized
uggcuccccagguaggauucga

SEQ ID NO: 268 - RNA, 19 nt, Artificial Sequence
  Artificially synthesized
uggcuccccagguaggauu

SEQ ID NO: 269 - RNA, 22 nt, Artificial Sequence
  Artificially synthesized
uaccaagcugcgcuacaucccu

SEQ ID NO: 270 - RNA, 19 nt, Artificial Sequence
  Artificially synthesized
caagcugcgcuacaucccu

SEQ ID NO: 271 - RNA, 22 nt, Artificial Sequence
  Artificially synthesized
uagggaugacaggauuugaacc

SEQ ID NO: 272 - RNA, 19 nt, Artificial Sequence
  Artificially synthesized
uagggaugacaggauuuga

SEQ ID NO: 273 - RNA, 22 nt, Artificial Sequence
  Artificially synthesized
cugaccacuagaccauggaacc

SEQ ID NO: 274 - RNA, 19 nt, Artificial Sequence
  Artificially synthesized
accacuagaccauggaacc

SEQ ID NO: 275 - RNA, 22 nt, Artificial Sequence
  Artificially synthesized
uggagguuccaccgagaccuga

SEQ ID NO: 276 - RNA, 19 nt, Artificial Sequence
  Artificially synthesized
uggagguuccaccgagacc

SEQ ID NO: 277 - RNA, 22 nt, Artificial Sequence
  Artificially synthesized
cuaaccgcuggacgacaacgga

SEQ ID NO: 278 - RNA, 19 nt, Artificial Sequence
  Artificially synthesized
accgcuggacgacaacgga

SEQ ID NO: 279 - RNA, 22 nt, Artificial Sequence
  Artificially synthesized
ugguuccguugccgggaaacga

SEQ ID NO: 280 - RNA, 19 nt, Artificial Sequence
  Artificially synthesized
ugguuccguugccgggaaa

SEQ ID NO: 281 - RNA, 22 nt, Artificial Sequence
  Artificially synthesized
auuuccauuauguuaccuagcc

SEQ ID NO: 282 - RNA, 19 nt, Artificial Sequence
  Artificially synthesized
uccauuauguuaccuagcc

SEQ ID NO: 283 - RNA, 22 nt, Artificial Sequence
  Artificially synthesized
uggaggccaaggacggggucga

SEQ ID NO: 284 - RNA, 19 nt, Artificial Sequence
  Artificially synthesized
uggaggccaaggacggggu

SEQ ID NO: 285 - RNA, 22 nt, Artificial Sequence
  Artificially synthesized
uugaccauuacucuaccccgcu

SEQ ID NO: 286 - RNA, 19 nt, Artificial Sequence
  Artificially synthesized
accauuacucuaccccgcu

SEQ ID NO: 287 - RNA, 22 nt, Artificial Sequence
  Artificially synthesized
uggaggcggggacaggauucga

SEQ ID NO: 288 - RNA, 19 nt, Artificial Sequence
  Artificially synthesized
uggaggcggggacaggauu

SEQ ID NO: 289 - RNA, 22 nt, Artificial Sequence
  Artificially synthesized
gaccagacugcgcuacaccucg

SEQ ID NO: 290 - RNA, 19 nt, Artificial Sequence
  Artificially synthesized
cagacugcgcuacaccucg

SEQ ID NO: 291 - RNA, 22 nt, Artificial Sequence
  Artificially synthesized
uggucaaggugacaggauucga

SEQ ID NO: 292 - RNA, 19 nt, Artificial Sequence
  Artificially synthesized
uggucaaggugacaggauu

SEQ ID NO: 293 - RNA, 22 nt, Artificial Sequence
  Artificially synthesized
ucuaccacuagaccacuggugc

SEQ ID NO: 294 - RNA, 19 nt, Artificial Sequence
  Artificially synthesized
accacuagaccacuggugc

SEQ ID NO: 295 - RNA, 22 nt, Artificial Sequence
  Artificially synthesized
uggugcaccagccgggaaacga

SEQ ID NO: 296 - RNA, 19 nt, Artificial Sequence
  Artificially synthesized
uggugcaccagccgggaaa

SEQ ID NO: 297 - RNA, 22 nt, Artificial Sequence
  Artificially synthesized
cuuaccacuauacuacaacgac

SEQ ID NO: 298 - RNA, 19 nt, Artificial Sequence
  Artificially synthesized
accacuauacuacaacgac

SEQ ID NO: 299 - RNA, 22 nt, Artificial Sequence
  Artificially synthesized
uggcgccguugccggggaucga

SEQ ID NO: 300 - RNA, 19 nt, Artificial Sequence
  Artificially synthesized
uggcgccguugccggggau

SEQ ID NO: 301 - RNA, 22 nt, Artificial Sequence
  Artificially synthesized
cuaccaacugagcuaaggucgg

SEQ ID NO: 302 - RNA, 19 nt, Artificial Sequence
  Artificially synthesized
ccaacugagcuaaggucgg

SEQ ID NO: 303 - RNA, 22 nt, Artificial Sequence
  Artificially synthesized
ugguccgaccagccggauucga

SEQ ID NO: 304 - RNA, 19 nt, Artificial Sequence
  Artificially synthesized
ugguccgaccagccggauu

SEQ ID NO: 305 - RNA, 22 nt, Artificial Sequence
  Artificially synthesized
cuaccaucugagcuacaucccc

SEQ ID NO: 306 - RNA, 19 nt, Artificial Sequence
  Artificially synthesized
ccaucugagcuacaucccc

SEQ ID NO: 307 - RNA, 22 nt, Artificial Sequence
  Artificially synthesized
ugguggagaugcgggguaucga

SEQ ID NO: 308 - RNA, 19 nt, Artificial Sequence
  Artificially synthesized
ugguggagaugcgggguau

SEQ ID NO: 309 - RNA, 22 nt, Artificial Sequence
  Artificially synthesized
ugaccaacuagacuacaacgga

SEQ ID NO: 310 - RNA, 19 nt, Artificial Sequence
  Artificially synthesized
ccaacuagacuacaacgga

SEQ ID NO: 311 - RNA, 22 nt, Artificial Sequence
  Artificially synthesized
ugguuccguugccgggacuuga

SEQ ID NO: 312 - RNA, 19 nt, Artificial Sequence
  Artificially synthesized
ugguuccguugccgggacu

SEQ ID NO: 313 - RNA, 22 nt, Artificial Sequence
  Artificially synthesized
cuaaccacuggacgaaagggac

SEQ ID NO: 314 - RNA, 19 nt, Artificial Sequence
  Artificially synthesized
accacuggacgaaagggac

SEQ ID NO: 315 - RNA, 22 nt, Artificial Sequence
  Artificially synthesized
ugguaccccuuacgggaaucga

SEQ ID NO: 316 - RNA, 19 nt, Artificial Sequence
  Artificially synthesized
ugguaccccuuacgggaau

SEQ ID NO: 317 - RNA, 22 nt, Artificial Sequence
  Artificially synthesized
cuauccacugagcuacaggcgc

SEQ ID NO: 318 - RNA, 19 nt, Artificial Sequence
  Artificially synthesized
uccacugagcuacaggcgc

SEQ ID NO: 319 - RNA, 22 nt, Artificial Sequence
  Artificially synthesized
uggcgcgccagguaggggucga

SEQ ID NO: 320 - RNA, 19 nt, Artificial Sequence
  Artificially synthesized
uggcgcgccagguaggggu

SEQ ID NO: 321 - RNA, 22 nt, Artificial Sequence
  Artificially synthesized
uaaccaacuacaccacgaaacc

SEQ ID NO: 322 - RNA, 19 nt, Artificial Sequence
  Artificially synthesized
ccaacuacaccacgaaacc

SEQ ID NO: 323 - RNA, 22 nt, Artificial Sequence
  Artificially synthesized
ugguggcuucgcccggguucga

SEQ ID NO: 324 - RNA, 19 nt, Artificial Sequence
  Artificially synthesized
ugguggcuucgcccggguu

SEQ ID NO: 325 - RNA, 22 nt, Artificial Sequence
  Artificially synthesized
uaaccgacuacaccacccagac

SEQ ID NO: 326 - RNA, 19 nt, Artificial Sequence
  Artificially synthesized
ccgacuacaccacccagac

SEQ ID NO: 327 - RNA, 22 nt, Artificial Sequence
  Artificially synthesized
uggugucugagcccggguucga

SEQ ID NO: 328 - RNA, 19 nt, Artificial Sequence
  Artificially synthesized
uggugucugagcccggguu

SEQ ID NO: 329 - RNA, 22 nt, Artificial Sequence
  Artificially synthesized
ccaaccgcucagacauccaucc

SEQ ID NO: 330 - RNA, 19 nt, Artificial Sequence
  Artificially synthesized
accgcucagacauccaucc

SEQ ID NO: 331 - RNA, 22 nt, Artificial Sequence
  Artificially synthesized
uggcggauggagagggauucga

SEQ ID NO: 332 - RNA, 19 nt, Artificial Sequence
  Artificially synthesized
uggcggauggagagggauu

SEQ ID NO: 333 - RNA, 22 nt, Artificial Sequence
  Artificially synthesized
cucccaacugagcuauccccgc

SEQ ID NO: 334 - RNA, 19 nt, Artificial Sequence
  Artificially synthesized
ccaacugagcuauccccgc

SEQ ID NO: 335 - RNA, 22 nt, Artificial Sequence
  Artificially synthesized
uggugcggugaacguggaucaa

SEQ ID NO: 336 - RNA, 19 nt, Artificial Sequence
  Artificially synthesized
uggugcggugaacguggau

SEQ ID NO: 337 - RNA, 22 nt, Artificial Sequence
  Artificially synthesized
uuaaccauucagccauggaugc

SEQ ID NO: 338 - RNA, 19 nt, Artificial Sequence
  Artificially synthesized
accauucagccauggaugc

SEQ ID NO: 339 - RNA, 22 nt, Artificial Sequence
  Artificially synthesized
uggugcauccaguaggaauuga

SEQ ID NO: 340 - RNA, 19 nt, Artificial Sequence
  Artificially synthesized
uggugcauccaguaggaau

SEQ ID NO: 341 - RNA, 22 nt, Artificial Sequence
  Artificially synthesized
ucuaccguugaguuagcaaccc

SEQ ID NO: 342 - RNA, 19 nt, Artificial Sequence
  Artificially synthesized
accguugaguuagcaaccc

SEQ ID NO: 343 - RNA, 22 nt, Artificial Sequence
  Artificially synthesized
ugguggguugcccgggauucga

SEQ ID NO: 344 - RNA, 19 nt, Artificial Sequence
  Artificially synthesized
ugguggguugcccgggauu

SEQ ID NO: 345 - RNA, 22 nt, Artificial Sequence
  Artificially synthesized
ucaaccacucagccaucucucc

SEQ ID NO: 346 - RNA, 19 nt, Artificial Sequence
  Artificially synthesized
accacucagccaucucucc

SEQ ID NO: 347 - RNA, 22 nt, Artificial Sequence
  Artificially synthesized
uggaggagagagagggauucga

SEQ ID NO: 348 - RNA, 19 nt, Artificial Sequence
  Artificially synthesized
uggaggagagagagggauu

SEQ ID NO: 349 - RNA, 22 nt, Artificial Sequence
  Artificially synthesized
caaccacucggccaucucuccu

SEQ ID NO: 350 - RNA, 19 nt, Artificial Sequence
  Artificially synthesized
ccacucggccaucucuccu

SEQ ID NO: 351 - RNA, 22 nt, Artificial Sequence
  Artificially synthesized
uggcggaaagagagggauucga

SEQ ID NO: 352 - RNA, 19 nt, Artificial Sequence
  Artificially synthesized
uggcggaaagagagggauu

SEQ ID NO: 353 - RNA, 22 nt, Artificial Sequence
  Artificially synthesized
uuuaccacuaaacuauacccgc

SEQ ID NO: 354 - RNA, 19 nt, Artificial Sequence
  Artificially synthesized
accacuaaacuauacccgc

SEQ ID NO: 355 - RNA, 22 nt, Artificial Sequence
  Artificially synthesized
uggagcggguagcgggaaucga

SEQ ID NO: 356 - RNA, 19 nt, Artificial Sequence
  Artificially synthesized
uggagcggguagcgggaau

SEQ ID NO: 357 - RNA, 22 nt, Artificial Sequence
  Artificially synthesized
cuauccauuagacaauggacgc

SEQ ID NO: 358 - RNA, 19 nt, Artificial Sequence
  Artificially synthesized
uccauuagacaauggacgc

SEQ ID NO: 359 - RNA, 22 nt, Artificial Sequence
  Artificially synthesized
uggugcguccaauaggauuuga

SEQ ID NO: 360 - RNA, 19 nt, Artificial Sequence
  Artificially synthesized
uggugcguccaauaggauu

SEQ ID NO: 361 - RNA, 22 nt, Artificial Sequence
  Artificially synthesized
uaauccucugagcuacaggccc

SEQ ID NO: 362 - RNA, 19 nt, Artificial Sequence
  Artificially synthesized
uccucugagcuacaggccc

SEQ ID NO: 363 - RNA, 22 nt, Artificial Sequence
  Artificially synthesized
uggugggcgaggagggauucga

SEQ ID NO: 364 - RNA, 19 nt, Artificial Sequence
  Artificially synthesized
uggugggcgaggagggauu

SEQ ID NO: 365 - RNA, 22 nt, Artificial Sequence
  Artificially synthesized
cuuaccacucggccauaucgcc

SEQ ID NO: 366 - RNA, 19 nt, Artificial Sequence
  Artificially synthesized
accacucggccauaucgcc

SEQ ID NO: 367 - RNA, 22 nt, Artificial Sequence
  Artificially synthesized
uggaggcgacacccggauuuga

SEQ ID NO: 368 - RNA, 19 nt, Artificial Sequence
  Artificially synthesized
uggaggcgacacccggauu

SEQ ID NO: 369 - RNA, 22 nt, Artificial Sequence
  Artificially synthesized
uuaaccgcucgggcaucgaccc

SEQ ID NO: 370 - RNA, 19 nt, Artificial Sequence
  Artificially synthesized
accgcucgggcaucgaccc

SEQ ID NO: 371 - RNA, 22 nt, Artificial Sequence
  Artificially synthesized
uggugggccgagcuggauuuga

SEQ ID NO: 372 - RNA, 19 nt, Artificial Sequence
  Artificially synthesized
uggugggccgagcuggauu

SEQ ID NO: 373 - RNA, 22 nt, Artificial Sequence
  Artificially synthesized
ucuaccgcugaguuaaaagggc

SEQ ID NO: 374 - RNA, 19 nt, Artificial Sequence
  Artificially synthesized
accgcugaguuaaaagggc

SEQ ID NO: 375 - RNA, 22 nt, Artificial Sequence
  Artificially synthesized
uggagccccuuaucggauuuga

SEQ ID NO: 376 - RNA, 19 nt, Artificial Sequence
  Artificially synthesized
uggagccccuuaucggauu

SEQ ID NO: 377 - RNA, 22 nt, Artificial Sequence
  Artificially synthesized
cuaaccucugagcuaagcaggc

SEQ ID NO: 378 - RNA, 19 nt, Artificial Sequence
  Artificially synthesized
accucugagcuaagcaggc

SEQ ID NO: 379 - RNA, 22 nt, Artificial Sequence
  Artificially synthesized
uggagccggcuaucggaaucga

SEQ ID NO: 380 - RNA, 19 nt, Artificial Sequence
  Artificially synthesized
uggagccggcuaucggaau

SEQ ID NO: 381 - RNA, 22 nt, Artificial Sequence
  Artificially synthesized
cuaaccacugaguuaaguaggu

SEQ ID NO: 382 - RNA, 19 nt, Artificial Sequence
  Artificially synthesized
accacugaguuaaguaggu

SEQ ID NO: 383 - RNA, 22 nt, Artificial Sequence
  Artificially synthesized
ugguaccuacuauuggauuuga

SEQ ID NO: 384 - RNA, 19 nt, Artificial Sequence
  Artificially synthesized
ugguaccuacuauuggauu

SEQ ID NO: 385 - RNA, 22 nt, Artificial Sequence
  Artificially synthesized
cuaccaauuccgccauaucccc

SEQ ID NO: 386 - RNA, 19 nt, Artificial Sequence
  Artificially synthesized
ccaauuccgccauaucccc

SEQ ID NO: 387 - RNA, 22 nt, Artificial Sequence
  Artificially synthesized
ugguggggauagagggacuuga

SEQ ID NO: 388 - RNA, 19 nt, Artificial Sequence
  Artificially synthesized
ugguggggauagagggacu

SEQ ID NO: 389 - RNA, 22 nt, Artificial Sequence
  Artificially synthesized
cuaccgauuucaccauagcggc

SEQ ID NO: 390 - RNA, 19 nt, Artificial Sequence
  Artificially synthesized
ccgauuucaccauagcggc

SEQ ID NO: 391 - RNA, 22 nt, Artificial Sequence
  Artificially synthesized
uggugccgccacucggacucga

SEQ ID NO: 392 - RNA, 19 nt, Artificial Sequence
  Artificially synthesized
uggugccgccacucggacu

SEQ ID NO: 393 - RNA, 22 nt, Artificial Sequence
  Artificially synthesized
cuaccagcugagcuaucccgac

SEQ ID NO: 394 - RNA, 19 nt, Artificial Sequence
  Artificially synthesized
ccagcugagcuaucccgac

SEQ ID NO: 395 - RNA, 22 nt, Artificial Sequence
  Artificially synthesized
uggugccaggaaccagauuuga

SEQ ID NO: 396 - RNA, 19 nt, Artificial Sequence
  Artificially synthesized
uggugccaggaaccagauu

SEQ ID NO: 397 - RNA, 22 nt, Artificial Sequence
  Artificially synthesized
uaccuaacugagcuauagcccu

SEQ ID NO: 398 - RNA, 19 nt, Artificial Sequence
  Artificially synthesized
cuaacugagcuauagcccu

SEQ ID NO: 399 - RNA, 22 nt, Artificial Sequence
  Artificially synthesized
ugguagggcuauacggacucgg

SEQ ID NO: 400 - RNA, 19 nt, Artificial Sequence
  Artificially synthesized
ugguagggcuauacggacu

SEQ ID NO: 401 - RNA, 22 nt, Artificial Sequence
  Artificially synthesized
ucuaccgcugaguuauaucccu

SEQ ID NO: 402 - RNA, 19 nt, Artificial Sequence
  Artificially synthesized
accgcugaguuauaucccu

SEQ ID NO: 403 - RNA, 22 nt, Artificial Sequence
  Artificially synthesized
ugguagggauaaucaggcucga

SEQ ID NO: 404 - RNA, 19 nt, Artificial Sequence
  Artificially synthesized
ugguagggauaaucaggcu

SEQ ID NO: 405 - RNA, 22 nt, Artificial Sequence
  Artificially synthesized
uaccgaacugaacuaagagcgc

SEQ ID NO: 406 - RNA, 19 nt, Artificial Sequence
  Artificially synthesized
cgaacugaacuaagagcgc

SEQ ID NO: 407 - RNA, 22 nt, Artificial Sequence
  Artificially synthesized
uggcacgcucuguaggauuuga

SEQ ID NO: 408 - RNA, 19 nt, Artificial Sequence
  Artificially synthesized
uggcacgcucuguaggauu

SEQ ID NO: 409 - RNA, 22 nt, Artificial Sequence
  Artificially synthesized
ucuaccacugagcuaauagccc

SEQ ID NO: 410 - RNA, 19 nt, Artificial Sequence
  Artificially synthesized
accacugagcuaauagccc

SEQ ID NO: 411 - RNA, 22 nt, Artificial Sequence
  Artificially synthesized
uggugggccauccuggacuuga

SEQ ID NO: 412 - RNA, 19 nt, Artificial Sequence
  Artificially synthesized
uggugggccauccuggacu

SEQ ID NO: 413 - RNA, 22 nt, Artificial Sequence
  Artificially synthesized
cuaccaacugagcuauaucccc

SEQ ID NO: 414 - RNA, 19 nt, Artificial Sequence
  Artificially synthesized
ccaacugagcuauaucccc

SEQ ID NO: 415 - RNA, 22 nt, Artificial Sequence
  Artificially synthesized
ugguggagauaagcggacucga

SEQ ID NO: 416 - RNA, 19 nt, Artificial Sequence
  Artificially synthesized
ugguggagauaagcggacu

SEQ ID NO: 417 - RNA, 22 nt, Artificial Sequence
  Artificially synthesized
cuaccaacugagcuauacaccc

SEQ ID NO: 418 - RNA, 19 nt, Artificial Sequence
  Artificially synthesized
ccaacugagcuauacaccc

SEQ ID NO: 419 - RNA, 22 nt, Artificial Sequence
  Artificially synthesized
ugguggguauagcaggacuuga

SEQ ID NO: 420 - RNA, 19 nt, Artificial Sequence
  Artificially synthesized
ugguggguauagcaggacu

SEQ ID NO: 421 - RNA, 22 nt, Artificial Sequence
  Artificially synthesized
ucuuccucugagcuacagggug

SEQ ID NO: 422 - RNA, 19 nt, Artificial Sequence
  Artificially synthesized
uccucugagcuacagggug

SEQ ID NO: 423 - RNA, 22 nt, Artificial Sequence
  Artificially synthesized
uggguaaccugguaggacuuga

SEQ ID NO: 424 - RNA, 19 nt, Artificial Sequence
  Artificially synthesized
uggguaaccugguaggacu

SEQ ID NO: 425 - RNA, 22 nt, Artificial Sequence
  Artificially synthesized
cuuaccacuuggcuauacucca

SEQ ID NO: 426 - RNA, 19 nt, Artificial Sequence
  Artificially synthesized
accacuuggcuauacucca

SEQ ID NO: 427 - RNA, 22 nt, Artificial Sequence
  Artificially synthesized
uggcuggaguaaaaggauucga

SEQ ID NO: 428 - RNA, 19 nt, Artificial Sequence
  Artificially synthesized
uggcuggaguaaaaggauu

SEQ ID NO: 429 - RNA, 22 nt, Artificial Sequence
  Artificially synthesized
caaccaauuaaacuauaagccc

SEQ ID NO: 430 - RNA, 19 nt, Artificial Sequence
  Artificially synthesized
ccaauuaaacuauaagccc

SEQ ID NO: 431 - RNA, 22 nt, Artificial Sequence
  Artificially synthesized
ugguaggcuuaguagggcucga

SEQ ID NO: 432 - RNA, 19 nt, Artificial Sequence
  Artificially synthesized
ugguaggcuuaguagggcu

SEQ ID NO: 433 - RNA, 22 nt, Artificial Sequence
  Artificially synthesized
uuaaccauucagccauggaugc

SEQ ID NO: 434 - RNA, 19 nt, Artificial Sequence
  Artificially synthesized
accauucagccauggaugc

SEQ ID NO: 435 - RNA, 22 nt, Artificial Sequence
  Artificially synthesized
uggugcauccaguaggaauuga

SEQ ID NO: 436 - RNA, 19 nt, Artificial Sequence
  Artificially synthesized
uggugcauccaguaggaau

SEQ ID NO: 437 - RNA, 22 nt, Artificial Sequence
  Artificially synthesized
uugaccacucggccacucuccc

SEQ ID NO: 438 - RNA, 19 nt, Artificial Sequence
  Artificially synthesized
accacucggccacucuccc

SEQ ID NO: 439 - RNA, 22 nt, Artificial Sequence
  Artificially synthesized
uggugggagaggcaggauucga

SEQ ID NO: 440 - RNA, 19 nt, Artificial Sequence
  Artificially synthesized
uggugggagaggcaggauu

SEQ ID NO: 441 - RNA, 22 nt, Artificial Sequence
  Artificially synthesized
uaagccacucagccauaccucc

SEQ ID NO: 442 - RNA, 19 nt, Artificial Sequence
  Artificially synthesized
gccacucagccauaccucc

SEQ ID NO: 443 - RNA, 22 nt, Artificial Sequence
  Artificially synthesized
uggcggaggaaaugggauucga

SEQ ID NO: 444 - RNA, 19 nt, Artificial Sequence
  Artificially synthesized
uggcggaggaaaugggauu

SEQ ID NO: 445 - RNA, 22 nt, Artificial Sequence
  Artificially synthesized
uaaccagcugagcuaccugaac

SEQ ID NO: 446 - RNA, 19 nt, Artificial Sequence
  Artificially synthesized
ccagcugagcuaccugaac

SEQ ID NO: 447 - RNA, 22 nt, Artificial Sequence
  Artificially synthesized
uggcgcuuagaaguggauucga

SEQ ID NO: 448 - RNA, 19 nt, Artificial Sequence
  Artificially synthesized
uggcgcuuagaaguggauu

SEQ ID NO: 449 - RNA, 22 nt, Artificial Sequence
  Artificially synthesized
acuuccauuauucgaucguuac

SEQ ID NO: 450 - RNA, 19 nt, Artificial Sequence
  Artificially synthesized
uccauuauucgaucguuac

SEQ ID NO: 451 - RNA, 22 nt, Artificial Sequence
  Artificially synthesized
uggaguaacgaauuggauuuga

SEQ ID NO: 452 - RNA, 19 nt, Artificial Sequence
  Artificially synthesized
uggaguaacgaauuggauu

SEQ ID NO: 453 - RNA, 22 nt, Artificial Sequence
  Artificially synthesized
uaccaagcugcgcuacaucccu

SEQ ID NO: 454 - RNA, 19 nt, Artificial Sequence
  Artificially synthesized
caagcugcgcuacaucccu

SEQ ID NO: 455 - RNA, 22 nt, Artificial Sequence
  Artificially synthesized
ugguagggaugacaggauugga

SEQ ID NO: 456 - RNA, 19 nt, Artificial Sequence
  Artificially synthesized
ugguagggaugacaggauu

SEQ ID NO: 457 - RNA, 22 nt, Artificial Sequence
  Artificially synthesized
ucuaccacugagcuaauagccc

SEQ ID NO: 458 - RNA, 19 nt, Artificial Sequence
  Artificially synthesized
accacugagcuaauagccc

SEQ ID NO: 459 - RNA, 22 nt, Artificial Sequence
  Artificially synthesized
uggugggccauccuggacuuga

SEQ ID NO: 460 - RNA, 19 nt, Artificial Sequence
  Artificially synthesized
uggugggccauccuggacu

SEQ ID NO: 461 - RNA, 22 nt, Artificial Sequence
  Artificially synthesized
ucuaccauuaagcuauuuccgc

SEQ ID NO: 462 - RNA, 19 nt, Artificial Sequence
  Artificially synthesized
accauuaagcuauuuccgc

SEQ ID NO: 463 - RNA, 22 nt, Artificial Sequence
  Artificially synthesized
uggagcggaaggagggacuuga

SEQ ID NO: 464 - RNA, 19 nt, Artificial Sequence
  Artificially synthesized
uggagcggaaggagggacu

SEQ ID NO: 465 - RNA, 77 nt, Panax ginseng
gcggauguagccaaguggaucaaggcaguggauugugaauccaccaugcgcggguucaauucccgucguucgcccca

SEQ ID NO: 466 - RNA, 74 nt, Panax ginseng
gcggauauagucgaaugguaaaauuucucuuugccaaggagaagacgcggguucgauucccgcuauccgcccca

SEQ ID NO: 467 - RNA, 84 nt, Panax ginseng
gccuugguggugaaaugguagacacgcgagacucaaaaucucgugcuaaagagcguggagguucgaguccucuucaaggcacca

SEQ ID NO: 468 - RNA, 77 nt, Panax ginseng
cgcggaguagagcaguuugguagcucgcaaggcucauaaccuugaggucacggguucaaauccugucuccgcaacca

SEQ ID NO: 469 - RNA, 77 nt, Panax ginseng
gggauuguaguucaaucggucagagcaccgcccugucaaggcggaagcugcggguucgagccccgucagucccgcca

SEQ ID NO: 470 - RNA, 91 nt, Panax ginseng
ggagagauggcugaguggacuaaagcggcggauugcuaauccgcuguacgaguuauucguaccgaggguucgaaucccucucuuuccgcca

SEQ ID NO: 471 - RNA, 75 nt, Panax ginseng
uggggcguggccaagugguaaggcaacggguuuuggucccgcuauucggagguucgaauccuuccgucccagcca

SEQ ID NO: 472 - RNA, 76 nt, Panax ginseng
gcccccaucgucuagugguucaggacaucucucuuucaaggaggcagcggggauucgacuuccccuggggguacca

SEQ ID NO: 473 - RNA, 75 nt, Panax ginseng
uccucaguagcucagugguagagcggucggcuguuaacugacuggucguagguucgaauccuaccuggggagcca

SEQ ID NO: 474 - RNA, 77 nt, Panax ginseng
agggauguagcgcagcuugguagcgcuuuuguuuuggguacaaaaugucacggguucaaauccugucaucccuacca

SEQ ID NO: 475 - RNA, 75 nt, Panax ginseng
gguuccauggucuaguggucaggacauuggacucugaauccaguaacccgaguucaggucucgguggaaccucca

SEQ ID NO: 476 - RNA, 75 nt, Panax ginseng
uccguugucguccagcgguuaggauaucuggcuuucacccaggagacccggguucguuucccggcaacggaacca

SEQ ID NO: 477 - RNA, 74 nt, Panax ginseng
ggcuagguaacauaauggaaauguauuggacugcaaauccuggaaugacgguucgaccccguccuuggccucca

SEQ ID NO: 478 - RNA, 76 nt, Panax ginseng
agcgggguagaguaauggucaacucaucagucucauuaucugaagacuacagguucgaauccuguccccgccucca

SEQ ID NO: 479 - RNA, 78 nt, Panax ginseng
cgagguguagcgcagucuggucagcgcaucuguuuuggguacagagggccauagguucgaauccugucaccuugacca

SEQ ID NO: 480 - RNA, 74 nt, Panax ginseng
gcaccaguggucuagugguagaauaguacccugccacgguacagacccggguucguuucccggcuggugcacca

SEQ ID NO: 481 - RNA, 75 nt, Panax ginseng
gucguuguaguauagugguaaguauucccgccugucacgcgggugacccggguucgauccccggcaacggcgcca

SEQ ID NO: 482 - RNA, 89 nt, Panax ginseng
ccgaccuuagcucaguugguagagcggaggacuguagugugcucguagcuauccuuaggucgcugguucgaauccggcuggucggacca

SEQ ID NO: 483 - RNA, 76 nt, Panax ginseng
ggggauguagcucagaugguagagcgcucgcuuagcaugcgagagguacggggaucgauaccccgcaucuccacca

SEQ ID NO: 484 - RNA, 76 nt, Panax ginseng
uccguuguagucuaguuggucaggauacucggcucucacccgagagacccggguucaagucccggcaacggaacca

SEQ ID NO: 485 - RNA, 75 nt, Panax ginseng
gucccuuucguccagugguuaggacaucgucuuuucaugucgaagacacggguucgauucccguaagggguacca

SEQ ID NO: 486 - RNA, 76 nt, Panax ginseng
gcgccuguagcucaguggauagagcgucuguuuccuaagcagaaagucguagguucgaccccuaccuggcgcgcca

SEQ ID NO: 487 - RNA, 77 nt, Panax ginseng
gguuucgugguguaguugguuaucacgucagccuaacacacugaaggucuccgguucgaacccgggcgaagccacca

SEQ ID NO: 488 - RNA, 77 nt, Panax ginseng
gucugggugguguagucgguuaucaugcuagucucacacacuagagguccccgguucgaacccgggcucagacacca

SEQ ID NO: 489 - RNA, 90 nt, Panax ginseng
ggauggaugucugagcgguuggaagagucggucuugaaaaccgaaguauugauaggaauaccggggguucgaaucccucuccauccgcca

SEQ ID NO: 490 - RNA, 76 nt, Panax ginseng
gcggggauagcucaguugggagagugucagacugaagaucuaaaggucacguguuugauccacguucaccgcacca

SEQ ID NO: 491 - RNA, 77 nt, Panax ginseng
gcauccauggcugaaugguuaaagcgcccaacucauaauuggcgaauucguagguucaauuccuacuggaugcacca

SEQ ID NO: 492 - RNA, 75 nt, Panax ginseng
ggguugcuaacucaacgguagaguacucggcuuuuaaccgacuaguuccggguucgaaucccgggcaacccacca

SEQ ID NO: 493 - RNA, 95 nt, Panax ginseng
ggagagauggcugagugguugauagcuccggucuugaaaaccggcauaguuuuaacaaagaacuaucgaggguucgaaucccucucucuccucca

SEQ ID NO: 494 - RNA, 91 nt, Panax ginseng
aggagagauggccgagugguugaaggcguagcauuggaacugcuauguaggcuuuuguuuaccgaggguucgaaucccucucuuuccgcca

SEQ ID NO: 495 - RNA, 74 nt, Panax ginseng
gcggguauaguuuagugguaaaacccuagccuuccaagcuaacgaugcggguucgauucccgcuacccgcucca

SEQ ID NO: 496 - RNA, 75 nt, Panax ginseng
gcguccauugucuaauggauaggacagaggucuucuaaaccuuugguauagguucaaauccuauuggacgcacca

SEQ ID NO: 497 - RNA, 77 nt, Panax ginseng
gggccuguagcucagaggauuagagcacguggcuacgaaccacggugucggggguucgaaucccuccucgcccacca

SEQ ID NO: 498 - RNA, 75 nt, Panax ginseng
ggcgauauggccgagugguaaggcgggggacugcaaauccuuuuuuccccaguucaaauccgggugucgccucca

SEQ ID NO: 499 - RNA, 87 nt, Panax ginseng
gggucgaugcccgagcgguuaauggggacggacuguaaauucguuggcaauaugucuacgcugguucaaauccagcucggcccacca

SEQ ID NO: 500 - RNA, 75 nt, Panax ginseng
gcccuuuuaacucagcgguagaguaacgccaugguaaggcguaagucaucgguucaaauccgauaaggggcucca

SEQ ID NO: 501 - RNA, 76 nt, Panax ginseng
gccugcuuagcucagagguuagagcaucgcauuuguaaugcgauggucaucgguucgauuccgauagccggcucca

SEQ ID NO: 502 - RNA, 76 nt, Panax ginseng
accuacuuaacucagugguuagaguauugcuuucauacggcgggagucauugguucaaauccaauaguagguacca

SEQ ID NO: 503 - RNA, 87 nt, Panax ginseng
ggggauauggcggaauugguagacgcuacggacuuaaaauccgucgacuuuaaaaucgugaggguucaagucccucuauccccacca

SEQ ID NO: 504 - RNA, 83 nt, Panax ginseng
gccgcuauggugaaaucgguagacacgcugcucuuaggaagcagugcuagagcaucucgguucgaguccgaguggcggcacca

SEQ ID NO: 505 - RNA, 76 nt, Panax ginseng
gucgggauagcucagcugguagagcagaggacugaaaauccucgugucaccaguucaaaucugguuccuggcacca

SEQ ID NO: 506 - RNA, 77 nt, Panax ginseng
agggcuauagcucaguuagguagagcaccucguuuacaccgagaaggucuacgguccgaguccguauagcccuacca

SEQ ID NO: 507 - RNA, 75 nt, Panax ginseng
agggauauaacucagcgguagagugucaccuugacgugguggaagucaucaguucgagccugauuaucccuacca

SEQ ID NO: 508 - RNA, 77 nt, Panax ginseng
gcgcucuuaguucaguucgguagaacgugggucuccaaaacccaaugucguagguucaaauccuacagagcgugcca

SEQ ID NO: 509 - RNA, 75 nt, Panax ginseng
gggcuauuagcucagugguagagcgcgccccugauaagggcgaggucucugguucaaguccaggauggcccacca

SEQ ID NO: 510 - RNA, 76 nt, Panax ginseng
ggggauauagcucaguugguagagcuccgcucuugcaaggcggaugucagcgguucgaguccgcuuaucuccacca

SEQ ID NO: 511 - RNA, 76 nt, Panax ginseng
ggguguauagcucaguugguagagcauugggcuuuuaaccuaauggucgcagguucaaguccugcuauacccacca

SEQ ID NO: 512 - RNA, 75 nt, Panax ginseng
cacccuguagcucagaggaagaguggucgucucuuagcugacaggucguagguucaaguccuaccagguuaccca

SEQ ID NO: 513 - RNA, 67 nt, Panax ginseng
uggaguauagccaagugguaaggcaccgguuuuugguaccgagguucgaauccuuuuacuccagcca

SEQ ID NO: 514 - RNA, 77 nt, Panax ginseng
gggcuuauaguuuaauugguugaaacguaccgcucauaacgguuauauuguagguucgagcccuacuaagccuacca

SEQ ID NO: 515 - RNA, 77 nt, Panax ginseng; identical to SEQ ID NO: 491

SEQ ID NO: 516 - RNA, 86 nt, Panax ginseng
gggagaguggccgaguggucaaaagcgacagacuguaaaucuguugaaguuuuucuacguagguucgaauccugccucucccacca

SEQ ID NO: 517 - RNA, 91 nt, Panax ginseng
ggagguauggcugaguggcuuaaggcauugguuugcuaaaucgacauacaagaagauuguaucauggguucgaaucccauuuccuccgcca

SEQ ID NO: 518 - RNA, 77 nt, Panax ginseng
guucagguagcucagcugguuagagcaaaggacugaaaauccuugugucagugguucgaauccacuucuaagcgcca

SEQ ID NO: 519 - RNA, 78 nt, Panax ginseng
guaacgaucgaauaauggaaguucacggggaaagucacuagacccgaagcauugguucaaauccaauucguuacucca

SEQ ID NO: 520 - RNA, 82 nt, Panax ginseng
agggauguagcgcagcuugguagcgccuuuguuuuggguaaaaaaugucacggguuccaauccaauccugucaucccuacca

SEQ ID NO: 521 - RNA, 75 nt, Panax ginseng; identical to SEQ ID NO: 509

SEQ ID NO: 522 - RNA, 75 nt, Panax ginseng
gcggaaauagcuuaaugguagagcauagccuugccaaggcugagguugaggguucaagucccuccuuccgcucca

(19) United States
(12) Patent Application Publication
(10) Pub. No.: US 2021/0052630 A1
(43) Pub. Date: Feb. 25, 2021

(54) Methods and Compositions for Preventing or Treating Heart Disease

(71) Applicant: Macau University of Science and Technology, Macau (CN)

(72) Inventors: Zhi-Hong JIANG, Macau (CN); Kua HU, Macau (CN); Tong-Meng YAN, Macau (CN)

(21) Appl. No.: 16/999,051

(22) Filed: Aug. 20, 2020

(30) Foreign Application Priority Data
Aug. 23, 2019 (CN) 201910784150.9

(51) Int. Cl. A61K 31/7105; A61K 47/54; A61P 9/00

(52) U.S. Cl. CPC A61K 31/7105; A61P 9/00; A61K 47/543

(57) ABSTRACT

Application of transfer RNA Molecules and their derived fragments for prevention or treatment of heart disease. The present invention provides a method of preventing or treating a subject suffering from heart diseases comprising administration of transfer RNA molecules and fragments derived from transfer RNA molecules or its functional variants or homologous to the subject, wherein the RNA molecules isolated from or derived from a plant of the genus Panax. The present invention also provides a pharmaceutical composition for the prevention or treatment of heart diseases comprising said effective amount of RNA molecule and a pharmaceutically tolerable vector, virus or excipient. The present invention provides a method for the prevention or treatment of a subject suffering from a heart disease. It is found that transfer RNA molecules from ginseng are particularly effective in the treatment of heart diseases, and also have a restorative effect on the myocardial cytoskeleton after ischemia-reperfusion injury.

### CROSS-REFERENCE TO RELATED APPLICATION

This application claims priority to, and the benefit of, Chinese Patent Application No. 20190784150.9 filed on Aug. 23, 2019. The entire contents of the foregoing application are hereby incorporated by reference for all purposes.

### REFERENCE TO SEQUENCE LISTING

This application contains a sequence listing which has been submitted electronically in ASCII format and is hereby incorporated by reference in its entirety. Said ASCII copy, created on Oct. 13, 2020, is named “M006_091_NPRUS_Sequence_list_revised.txt” and is 104468 bytes in size.

### TECHNICAL FIELD

The present invention belongs to the field of biomedicine, relating to a method of preventing or treating a subject suffering from heart disease comprising administration of transfer RNA molecules isolated from or derived from a plant of the genus Panax to the subject. The invention further relates to a pharmaceutical composition comprising a nucleic acid for the treatment and use thereof.

### BACKGROUND OF THE INVENTION

Coronary heart disease (CHD) has become the top leading cause of mortality and morbidity worldwide. Traditional Chinese medicines (TCMs) have been widely applied for preventing or treating CHD whereas lots of research efforts have been contributed to investigate the effectiveness of isolated small molecules such as saponins, terpenoids, flavonoids or the like in treating CHD. Some ginsenosides have been found to have effect in protecting cardiomyocytes exposed to hypoxia/reoxygenation in vitro. However, most of them are often toxic to human. Also, macromolecules such as DNAs, RNAs, and proteins are generally considered unstable and have poor effect in living human body and therefore have not been widely considered as suitable in said treatment.

Currently, some studies show that non-coding RNAs (ncRNAs) such as microRNAs have diverse regulatory roles through targeting different aspects of RNA transcription or post-transcription process in nearly all eukaryotic organisms. Lin Zhang et al. (Cell research 2012, 22, 107-126) suggested that exogenous plant microRNAs in foods could be taken up by the mammalian gastrointestinal (GI) tract and entering into the circulation to various organs, where they are capable of regulating the expression of mammalian genes. Goodarzi, H. et al. (Cell 2015, 161 (4), 790-802) revealed that endogenous tRNA derived fragments could suppress the stability of multiple oncogenic transcripts in breast cancer cells through binding and antagonizing activities of pathogenesis-related RNA-binding proteins. Nevertheless, there still remains a need to derive effective molecules from various sources such as plants for treatments.

Panax ginseng C. A. Mey, a species from the family of Araliaceae, is considered to be the most precious herbs distributed mountainous regions of China and Korea. The roots of P. ginseng have been a famous traditional Chinese medicine used worldwide for thousands of years to be a tonic to invigorate weak bodies. In addition, the main component of P. ginseng such as ginsenosides and polysaccharides had been proved to show significant effects on cardioprotection. However, the dosage of these components is massive, which may cause toxicity to human bodies. Therefore, there remains a continuing need for new and improved treatments for patients with CHD and/or associated with different complications.

### SUMMARY OF THE INVENTION

Therefore, in view of the inadequacy of existing technology, the purpose of the present invention is to provide transfer RNA molecules isolated from or derived from plant of genus Panax in the preparation of drugs for the prevention or treatment of heart diseases. Specifically, the purpose of the present invention is to identify or discover the key role of transfer RNA molecules isolated from or derived from plant of genus Panax in treatment of myocardial ischemia reperfusion, myocardial infarction, coronary heart disease, myocardial fibrosis and other cardiac diseases, and further application of diagnosis and treatment of these heart diseases.

The purpose of the invention is realized through the following technical scheme.

In a first aspect, the invention provides transfer RNA molecules and fragments derived from transfer RNA or its functional variants or homologous in the preparation of drugs for the prevention or treatment of a subject suffering from heart diseases, wherein said RNA molecule isolated from or derived from a plant of the genus Panax.

In an embodiment, the plant of the genus Panax comprises Panax ginseng C. A. Mey, Panax notoginseng (Burkill) F. H. Chen or Panax quinquefolius Linn. Preferably, said plant of the genus Panax is Panax ginseng C. A. Mey.

Preferably, the transfer RNA molecule comprises a sequence selected from SEQ ID NO: 465 to SEQ ID NO: 522.

In an alternative embodiment, the fragments derived from transfer RNA molecule is a double-stranded RNA molecule comprising a sense sequence selected from SEQ ID NO: 1 to SEQ ID NO: 232 or a functional variant or homologue therefore, and a complementary antisense sequence.

Preferably, the fragments derived from transfer RNA molecule is a double-stranded RNA molecule comprising a sense sequence selected from SEQ ID NO: 1 to SEQ ID NO: 40 or a functional variant or homologue therefore, and a complementary antisense sequence.

Wherein, said complementary antisense sequences of nucleotide sequences shown in any of SEQ ID NO:1 to SEQ ID NO:232 are showed in any of SEQ ID NO:233 to SEQ ID NO:464.

Preferably, the transfer RNA molecules and fragments derived from transfer RNA or its functional variants or homologous contains a 2 mer of 3′ overhang.

Preferably, the transfer RNA molecules and fragments derived from transfer RNA or its functional variants or homologous contains a 3′ cholesterol conjugation.

Preferably, the double-stranded RNA molecule comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

In an embodiment, said heart diseases are selected from one or more of angina pectoris, myocardial infarction, myocardial ischemic injury, coronary heart disease, cardiac hypertrophy, and myocardial fibrosis.

In an embodiment, the RNA molecule of the invention is a non-coding molecule has a sequence length of from about 50 to 200 nucleotides or 10 to 30 base pairs.

In another aspect, the invention provides a pharmaceutical composition for preventing or treating heart diseases comprising an effective amount of transfer RNA molecule, fragments derived from transfer RNA molecules or its functional variants or homologous and a pharmaceutically tolerable vector, virus or excipient, wherein the RNA molecule is isolated or derived from a plant of the genus Panax.

In an embodiment, the pharmaceutically tolerable vector selected from one or more of the gene delivery vectors, chitosan, cholesterol, liposomes and nanoparticles.

Preferably, transfer RNA molecules, fragments derived from transfer RNA molecule or its functional variants or homologous are provided as composition containing a gene delivery vector.

Preferably, the pharmaceutical composition is provided by intravenous, intramuscular, intracoronary or direct myocardial injection.

In an embodiment, the pharmaceutical composition comprising the RNA molecule isolated or derived from the plant of the genus Panax comprises Panax ginseng C. A. Mey, Panax notoginseng (Burkill) F. H. Chen or Panax quinquefolius Linn. Preferably, said plant of the genus Panax is Panax ginseng C. A. Mey.

In an embodiment, wherein the transfer RNA molecule comprises a sequence selected from SEQ ID NO: 465 to SEQ ID NO: 522.

In an alternative embodiment, the fragments derived from transfer RNA molecule is a double-stranded RNA molecule comprising a sense sequence selected from SEQ ID NO: 1 to SEQ ID NO: 232 or a functional variant or homologue therefore, and a complementary antisense sequence.

Preferably, the fragments derived from transfer RNA molecule is a double-stranded RNA molecule comprising a sense sequence selected from SEQ ID NO: 1 to SEQ ID NO: 40 or a functional variant or homologue therefore, and a complementary antisense sequence.

Wherein, said complementary antisense sequences of nucleotide sequences shown in any of SEQ ID NO:1 to SEQ ID NO:232 are showed in any of SEQ ID NO:233 to SEQ ID NO:464.

Preferably, the transfer RNA molecules and fragments derived from transfer RNA or its functional variants or homologous comprises a 2 mer of 3′ overhang.

Preferably, the transfer RNA molecules and fragments derived from transfer RNA or its functional variants or homologous comprises a 3′ cholesterol conjugation.

Preferably, the transfer RNA molecules and fragments derived from transfer RNA or its functional variants or homologous comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

In an embodiment, said heart diseases are selected from one or more of angina pectoris, myocardial infarction, myocardial ischemic injury, coronary heart disease, cardiac hypertrophy, and myocardial fibrosis.

In an embodiment, the RNA molecule of the invention is a non-coding molecule has a sequence length of from about 50 to 200 nucleotides or 10 to 30 base pairs.

In a further aspect, the invention provides a method of preventing or treating a subject suffering from heart diseases, said method comprises the step of administering of an effective amount of transfer RNA molecules and fragments derived from transfer RNA or its functional variants or homologous thereof.

In an embodiment, said method comprising a step of contacting said cardiomyocytes with an effective amount of transfer RNA molecules and fragments derived from transfer RNA or its functional variants or homologous which are isolated or derived from a plant of the genus Panax.

In an embodiment, said plant of the genus Panax comprises Panax ginseng C. A. Mey, Panax notoginseng (Burkill) F. H. Chen or Panax quinquefolius Linn. Preferably, said plant of the genus Panax is Panax ginseng C. A. Mey.

Preferably, the transfer RNA molecule comprises a sequence selected from SEQ ID NO: 465 to SEQ ID NO: 522.

In an alternative embodiment, the fragments derived from transfer RNA molecule is a double-stranded RNA molecule comprising a sense sequence selected from SEQ ID NO: 1 to SEQ ID NO: 232 or a functional variant or homologue therefore, and a complementary antisense sequence.

Preferably, the fragments derived from transfer RNA molecule is a double-stranded RNA molecule comprising a sense sequence selected from SEQ ID NO: 1 to SEQ ID NO: 40 or a functional variant or homologue therefore, and a complementary antisense sequence.

Wherein, said complementary antisense sequences of nucleotide sequences shown in any of SEQ ID NO:1 to SEQ ID NO:232 are showed in any of SEQ ID NO:233 to SEQ ID NO:464.

Preferably, the transfer RNA molecules and fragments derived from transfer RNA or its functional variants or homologous contains a 2 mer of 3′ overhang.

Preferably, the transfer RNA molecules and fragments derived from transfer RNA or its functional variants or homologous contains a 3′ cholesterol conjugation.

Preferably, the double-stranded RNA molecule comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

In an embodiment, said heart diseases are selected from one or more of angina pectoris, myocardial infarction, myocardial ischemic injury, coronary heart disease, cardiac hypertrophy, and myocardial fibrosis.

In an embodiment, the RNA molecule of the invention is a non-coding molecule has a sequence length of from about 50 to 200 nucleotides or 10 to 30 base pairs.

Still further, the invention provides a recombinant vector comprising the double-stranded RNA molecule, wherein the double-stranded RNA molecule comprising a sense sequence selected from SEQ ID NO: 1 to SEQ ID NO: 232 or a functional variant or homologue therefore, and a complementary antisense sequence.

Preferably, the double-stranded RNA molecule comprising a sense sequence selected from SEQ ID NO: 1 to SEQ ID NO: 40 or a functional variant or homologue therefore, and a complementary antisense sequence.

Preferably, the double-stranded RNA molecule comprises a 2 mer of 3′ overhang.

Preferably, the double-stranded RNA molecule comprises a 3′ cholesterol conjugation.

Preferably, the double-stranded RNA molecule comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

Further, the invention provides the application in the preparation of drugs for the prevention or treatment of heart disease, wherein the drug comprises the transfer RNA molecule, fragments derived from transfer RNA molecule or its functional variant or homologous, the pharmaceutical composition and the recombinant vector.

The invention provides a novel and effective approach for treating heart diseases by administration of RNA molecules that are isolated or derived from a plant of the genus Panax, or in particular double-stranded RNA molecules comprising a sequence selected from SEQ ID NO: 1 to 232. Administration of said RNA molecules is also suitable for promoting the growth and proliferation of cardiomyocytes.

The inventors have found that non-coding RNA molecules isolated from a plant of the genus Panax, particularly transfer RNA molecules, and RNA molecules derived from Panax are particularly useful in treatment of heart diseases. The RNA molecules with a sequence length of about 10 to 200 nucleotides are highly effective at promoting the growth and proliferation of cardiomyocytes. Besides, said RNA molecules have restorative effects on the myocardial cytoskeleton after ischemia-reperfusion injury.

Those skilled in the art will appreciate that the invention described herein is susceptible to variations and modifications other than those specifically described. The invention includes all such variations and modifications. The invention also includes all steps and features referred to or indicated in the specification, individually or collectively, and any and all combinations of the steps or features.

Other features and aspects of the invention will become apparent by consideration of the following detailed description and accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The details about the implementation plan of the invention are elaborated in combination with the attached figures.

FIG. 1 shows gel electrophoresis profiles of RNA molecules from Panax ginseng C. A. Mey, including low range ssRNA Ladder (denoted as “L”), small RNA molecules (denoted as “S”), transfer RNAs (denoted as “T”), and individual transfer RNA including tRNA^{Gly(GCC)}, tRNA^{His(GUG)}, tRNA^{Met(CAU)} (denoted as “Gly, His, Met” respectively), in accordance with an example embodiment.

FIG. 2 is a bar chart showing read length distribution of transfer RNAs from Panax ginseng C. A. Mey in accordance with an example embodiment.

FIG. 3A is a bar chart showing the cardiomyocytes proliferation of 300 nM RNA molecules, tRNA^{Gly(GCC)}, tRNA^{His(GUG)}, tRNA^{Met(CAU)} and tRNA^{Leu(CAA)} from Panax ginseng C. A. Mey on H9C2 cell line exposed to hypoxia injury, compared to a control group, a hypoxia group in accordance with an example embodiment (mean±SD n=2; ****, p<0.0001 vs. vehicle hypoxia; ####, p<0.0001 vs. vehicle control).

FIG. 3B is a bar chart showing the cardiomyocytes proliferation of 50 nM RNA molecules tRNA^{Gly(GCC)}, tRNA^{His(GUG)}, tRNA^{Met(CAU)} and tRNA^{Leu(CAA)} from Panax ginseng C. A. Mey on H9C2 cell line exposed to hypoxia/reoxygenation (H/R) injury, compared to a control group, a H/R group in accordance with an example embodiment (mean±SD n=3; *, p<0.05 vs. vehicle H/R; ####, p<0.0001 vs. vehicle control).

FIG. 4A is a bar chart showing the cell viability of H9C2 cells after treatment with a RNA molecule tRNA^{His(GUG)} at different concentrations, i.e. 100 nM, 50 nM, 25 nM, and 12.5 nM, compared to a control group, a H/R group in accordance with an example embodiment (mean±SD n=3; **, p<0.01, ***, p<0.001, ****, p<0.001 vs. vehicle H/R; ####, p<0.0001 vs. vehicle control).

FIG. 4B is a bar chart showing the cell viability of H9C2 cells after treatment with a RNA molecule tRNA^{Gly(GCC)} at different concentrations, i.e. 100 nM, 50 nM, 25 nM, and 12.5 nM, compared to a control group, a H/R group in accordance with an example embodiment (mean±SD n=3; *, p<0.05, **, p<0.01, ****, p<0.001 vs. vehicle H/R; ####, p<0.0001 vs. vehicle control).

FIG. 4C is a bar chart showing the cell viability of H9C2 cells after treatment with Ginsenosides Rg1 at different concentrations, i.e. 100 μM, 25 μM, 6.25 μM, and 1.56 μM, compared to a control group, a H/R group in accordance with an example embodiment (mean±SD n=3; ****, p<0.0001 vs. vehicle H/R; ####, p<0.0001 vs. vehicle control).

FIG. 5A is a bar chart showing the cell viability of H9C2 cells exposed to hypoxia/reoxygenation (H/R) injury after treatment with different RNA molecules derived from Panax ginseng C. A. Mey with a sequence length of 22 bp at a dose of 300 nM, compared to a control group, a H/R group in accordance with an example embodiment (mean±SD n=6; *, p<0.05 vs. vehicle H/R; ####, p<0.0001 vs. vehicle control).

FIG. 5B is a bar chart showing the cell viability of H9C2 cells exposed to hypoxia/reoxygenation (H/R) injury after treatment with different RNA molecules derived from Panax ginseng C. A. Mey with a sequence length of 19 bp at a dose of 300 nM, compared to a control group, a H/R group in accordance with an example embodiment (mean±SD n=6; *, p<0.05 vs. vehicle H/R; ####, p<0.0001 vs. vehicle control).

FIG. 6A is a bar chart showing the cell viability of H9C2 cells exposed to hypoxia/reoxygenation (H/R) injury after treatment with RNA molecule HC50 at different concentrations, i.e. 900 nM, 300 nM, 30 nM, 3 nM and 0.3 nM, compared to a control group, a H/R group in accordance with an example embodiment (mean±SD n=3; *, p<0.05, ****, p<0.0001 vs. vehicle H/R; ####, p<0.0001 vs. vehicle control).

FIG. 6B is a bar chart showing the cell viability of H9C2 cells exposed to hypoxia/reoxygenation (H/R) injury after treatment with RNA molecule HC83 at different concentrations, i.e. 900 nM, 300 nM, 30 nM, 3 nM and 0.3 nM, compared to a control group, a H/R group in accordance with an example embodiment (mean±SD n=3; **, p<0.01, ****, p<0.0001 vs. vehicle H/R; ####, p<0.0001 vs. vehicle control).

FIG. 7A is a bar chart showing the mitochondrial viability of H9C2 cells exposed to hypoxia/reoxygenation (H/R) injury after treatment with RNA molecule HC50 at different concentrations, i.e. 300 nM, 100 nM, 30 nM, and 3 nM, compared to a control group, a H/R group in accordance with an example embodiment (mean±SD n=3; *, p<0.05 vs. vehicle H/R; ####, p<0.0001 vs. vehicle control).

FIG. 7B is a bar chart showing the mitochondrial viability of H9C2 cells exposed to hypoxia/reoxygenation (H/R) injury after treatment with RNA molecule HC83 at different concentrations, i.e. 300 nM, 100 nM, 30 nM, and 3 nM, compared to a control group, a H/R group in accordance with an example embodiment (mean±SD n=3; ***, p<0.001, vs. vehicle H/R; ####, p<0.0001 vs. vehicle control).

FIG. 8A is a cytoskeleton image showing protective effects on cytoskeleton destruction of H9C2 cells caused by hypoxia/reoxygenation (H/R) injury after treatment with RNA molecule HC50 at different concentrations, i.e. 900 nM, 300 nM, 100 nM, 30 nM, 10 nM, 3 nM, and 0.3 nM compared to a control group, a H/R group in accordance with an example embodiment.

FIG. 8B is a cytoskeleton image showing protective effects on cytoskeleton destruction of H9C2 cells caused by hypoxia/reoxygenation (H/R) injury after treatment with RNA molecule HC83 at different concentrations, i.e. 900 nM, 300 nM, 100 nM, 30 nM, 10 nM, 3 nM, and 0.3 nM compared to a control group, a H/R group in accordance with an example embodiment.

FIG. 9A is a bar chart showing the cell viability of H9C2 cells exposed to hypoxia/reoxygenation (H/R) injury after treatment with cholesterol-conjugated RNA molecule HC50 at different concentrations, i.e. 900 nM, 300 nM, 30 nM, 3 nM and 0.3 nM, compared to a control group, a H/R group in accordance with an example embodiment (mean±SD n=3; *, p<0.05, ****, p<0.0001 vs. vehicle H/R; ####, p<0.0001 vs. vehicle control).

FIG. 9B is a bar chart showing the cell viability of H9C2 cells exposed to hypoxia/reoxygenation (H/R) injury after treatment with cholesterol-conjugated RNA molecule HC83 at different concentrations, i.e. 900 nM, 300 nM, 30 nM, 3 nM and 0.3 nM, compared to a control group, a H/R group in accordance with an example embodiment (mean±SD n=3; *, p<0.05, ***, p<0.001, ****, p<0.0001 vs. vehicle H/R; ####, p<0.0001 vs. vehicle control).

FIG. 10A is a cytoskeleton image showing protective effects on cytoskeleton destruction of H9C2 cells caused by hypoxia/reoxygenation (H/R) injury after treatment with cholesterol-conjugated RNA molecule HC50 at different concentrations, i.e. 900 nM, 300 nM, 100 nM, 30 nM, 10 nM and 3 nM compared to a control group, a H/R group in accordance with an example embodiment.

FIG. 10B is a cytoskeleton image showing protective effects on cytoskeleton destruction of H9C2 cells caused by hypoxia/reoxygenation (H/R) injury after treatment with cholesterol-conjugated RNA molecule HC83 at different concentrations, i.e. 900 nM, 300 nM, 100 nM, 30 nM, 10 nM and 3 nM compared to a control group, a H/R group in accordance with an example embodiment.

FIG. 11A is a bar chart showing the cell viability of H9C2 cells exposed to hypoxia/reoxygenation (H/R) injury after treatment with RNA molecule HC50 at different concentrations, i.e. 300 nM, 100 nM and 30 nM, by transfected with DharmaFECT4 transfection reagent, compared to a control group, a H/R along with DharmaFECT4 treated group in accordance with an example embodiment (mean±SD n=3; **, p<0.01, vs. vehicle H/R+ DharmaFECT4; ####, p<0.0001 vs. vehicle control).

FIG. 11B is a bar chart showing the cell viability of H9C2 cells exposed to hypoxia/reoxygenation (H/R) injury after treatment with RNA molecule HC83 at different concentrations, i.e. 300 nM, 100 nM and 30 nM, by transfected with DharmaFECT4 transfection reagent, compared to a control group, a H/R along with DharmaFECT4 treated group in accordance with an example embodiment (mean±SD n=3; *, p<0.05, vs. vehicle H/R+ DharmaFECT4; ####, p<0.0001 vs. vehicle control).

### DETAILED DESCRIPTION OF THE EMBODIMENTS

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usually and unavoidable impurities such as side products and components usually resulting from the respective preparation or method for obtaining the material such as traces of further components or solvents. “Consisting of” means that the material solely consists of, i.e. is formed by the respective element. As used herein, the forms “a,” “an,” and “the,” are intended to include the singular and plural forms unless the context clearly indicates otherwise.

The present invention in the first aspect provides a method of preventing or treating a subject suffering from heart disease comprising administration of transfer RNA molecules and fragments derived from transfer RNA molecules or its functional variants or homologous to the subject, wherein the RNA molecules isolated from or derived from a plant of the genus Panax. The RNA molecule administered according to the present invention may be naturally present, modified or artificially synthesized according to the sequences disclosed in the present invention, and preferably the RNA molecule is isolated or derived from a plant of the genus Panax. The RNA molecule of the present invention is not provided in the form of boiled extract obtained from the plant such as decoction, as it would be appreciated that RNA molecule is susceptible to spontaneous degradation at elevated temperature, alkaline pH, and the presence of nucleases or divalent metal ions.

The RNA molecule of the present invention has a sequence length of from about 10 to 200 nucleotides which can be regarded as a small RNA molecule. Preferably, the RNA molecule has a sequence length of from about 50 to about 200 nucleotides, from about 60 to about 150 nucleotides, in particular from about 70 to about 100 nucleotides.

The RNA molecule of the present invention comprises a sequence selected from SEQ ID NO: 1 to SEQ ID NO: 232 or a functional variant or homologue thereof. The term “functional variant” of the RNA molecule refers to a molecule substantially similar to said RNA molecule with one or more sequence alterations that do not affect the biological activity or function of the RNA molecule. The alterations in sequence that do not affect the functional properties of the resultant RNA molecules are well known in the art. For example, nucleotide changes which result in alteration of the -5′-terminal and -3′-terminal portions of the molecules would not be expected to alter the activity of the polynucleotides. In an embodiment, the RNA molecule of the present invention comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

In particular, the functional variant of the RNA molecule has at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the non-variant RNA molecule according to the present invention.

The term “homologue” used herein refers to nucleotides having a sequence identity of at least 50%, at least 60%, at least 70%, at least 80%, at least 90% or at least 95% to the RNA molecules according to the present invention. In an embodiment, the homologue of the RNA molecule has at least 90%, 91%, 92%, 93%, 94%, 95%, 96%, 97%, 98%, or 99% overall sequence identity to the RNA molecule.

In an embodiment, the RNA molecule is a non-coding molecule preferably selected from a transfer RNA molecule, a micro RNA molecule or a siRNA molecule; and more preferably is a transfer RNA molecule. tRNA molecules are highly conserved RNAs with function in various cellular processes such as reverse transcription, porphyrin biosynthesis or the like. In a particular embodiment, the RNA molecule of the invention comprises a sequence selected from SEQ ID NO: 465 to SEQ ID NO: 522 or a functional variant or homologue thereof; or the RNA molecule comprises SEQ ID NO: 465 to SEQ ID NO: 468 or a functional variant or homologue thereof; or the RNA molecule consists of a sequence selected from SEQ ID NO: 465 to SEQ ID NO: 522 or SEQ ID NO: 465 to SEQ ID NO: 468 or a functional variant or homologue thereof.

In an alternative embodiment where the RNA molecule is a small RNA molecule having a sequence length of from about 10 to about 30 base pairs, from about 15 to about 25 base pairs, from about 19 to about 22 base pairs, 19 base pairs or 22 base pairs. The RNA molecule comprises a sequence selected from SEQ ID NO: 1 to SEQ ID NO: 232 or a functional variant or homologue thereof, in particular SEQ ID NO: 1 to SEQ ID NO: 40 or a functional variant or homologue thereof; or consists of a sequence selected from SEQ ID NO: 1 to SEQ ID NO: 232, in particular SEQ ID NO: 1 to SEQ ID NO: 40 or a functional variant or homologue thereof.

Preferably, the RNA molecule is a double-stranded RNA molecule having a sense sequence selected from SEQ ID NO: 1 to SEQ ID NO: 232 or a functional variant or homologue thereof, and a complementary antisense sequence.

The antisense sequence is complementary to the sense sequence and therefore the antisense sequence is preferably selected from SEQ ID NO: 233 to 464 or functional variant or homologue thereof. In a particular embodiment, the double-stranded RNA molecule of the present invention has a sense sequence selected from SEQ ID NO: 1 to SEQ ID NO: 40 or a functional variant or homologue thereof, and a complementary antisense sequence selected from SEQ ID NO: 233 to SEQ ID NO: 272 or a functional variant or homologue thereof. The inventors unexpectedly found that the double-stranded RNA molecules of the present invention are particularly useful in treatment of heart diseases as described in detail below.

The RNA molecule of the present invention is preferably isolated or derived from the plant of the genus Panax. The plant of the genus Panax includes but is not limited to Panax ginseng C. A. Mey, Panax quinquefolius Linn., Panax notoginseng (Burkill) F. H. Chen, Panax pseudoginseng Wall, Panax zingiberensis C. Y. Wu et K. M. Feng. The plant of the genus Panax may be the source of Ginsenosides Rg1. In an embodiment, the RNA molecule is isolated or derived from Panax ginseng C. A. Mey.

In more detail, the RNA molecule of the present invention is preferably isolated or derived from the different plant organs of the genus Panax. The plant organs of the genus Panax includes but is not limited to leaves, roots, and fruits. In an embodiment, the RNA molecule is isolated or derived from the roots of Panax ginseng C. A. Mey.

In more detail, the preferred sequences of the RNA molecules of the present invention are listed in Tables 1 and 2 below. In an embodiment, RNA molecules of SEQ ID NO: 465 to SEQ ID NO: 522 as shown in Table 1 are isolated from a plant of genus Panax in particular from Panax ginseng C. A. Mey. These sequences are obtained by extraction, RNA isolation and purification of the plant. The inventors determined these RNA molecules are associated with chloroplasts, cytoplast and mitochondria. One possible approach to obtain the RNA molecules from a particular plant Panax ginseng C. A. Mey is illustrated in Example 1. It would be appreciated that other suitable methods for obtaining the isolated and purified RNA molecules of the present invention according to the disclosure herein can be applied, and the methods can be subject to appropriate modification to obtain an improved yield of the RNA molecules, without departing from the scope of the present invention.

TABLE 1
RNA molecules in particular tRNAs isolated from Panax ginseng C. A. Mey
according to the present invention.

| SEQ ID | | | Length |
|---|---|---|---|
| NO. | tRNA(s) | Sequence (5′ to 3′) | (mer) |
| 465 | tRNA^{His(GUG)} | GCGGAUGUAGCCAAGUGGAUCAAGGCAGUGGAUUGUGAA | 77 |
| | | UCCACCAUGCGCGGGUUCAAUUCCCGUCGUUCGCCCCA | |
| 466 | tRNA^{Gly(GCC)}_1 | GCGGAUAUAGUCGAAUGGUAAAAUUUCUCUUUGCCAAGGA | 74 |
| | | GAAGACGCGGGUUCGAUUCCCGCUAUCCGCCCCA | |
| 467 | tRNA^{Leu(CAA)} | GCCUUGGUGGUGAAAUGGUAGACACGCGAGACUCAAAAU | 84 |
| | | CUCGUGCUAAAGAGCGUGGAGGUUCGAGUCCUCUUCAAG | |
| | | GCACCA | |
| 468 | tRNA^{Met(CAU)}_1 | CGCGGAGUAGAGCAGUUUGGUAGCUCGCAAGGCUCAUAA | 77 |
| | | CCUUGAGGUCACGGGUUCAAAUCCUGUCUCCGCAACCA | |
| 469 | tRNA^{Asp(GUC)} | GGGAUUGUAGUUCAAUCGGUCAGAGCACCGCCCUGUCAA | 77 |
| | | GGCGGAAGCUGCGGGUUCGAGCCCCGUCAGUCCCGCCA | |
| 470 | tRNA^{Ser(GCU)}_1 | GGAGAGAUGGCUGAGUGGACUAAAGCGGCGGAUUGCUAA | 91 |
| | | UCCGCUGUACGAGUUAUUCGUACCGAGGGUUCGAAUCCC | |
| | | UCUCUUUCCGCCA | |
| 471 | tRNA^{Gln(UUG)}_1 | UGGGGCGUGGCCAAGUGGUAAGGCAACGGGUUUUGGUCC | 75 |
| | | CGCUAUUCGGAGGUUCGAAUCCUUCCGUCCCAGCCA | |
| 472 | tRNA^{Glu(UUC)}_1 | GCCCCCAUCGUCUAGUGGUUCAGGACAUCUCUCUUUCAA | 76 |
| | | GGAGGCAGCGGGGAUUCGACUUCCCCUGGGGGUACCA | |
| 473 | tRNA^{Asn(GUU)} | UCCUCAGUAGCUCAGUGGUAGAGCGGUCGGCUGUUAACU | 75 |
| | | GACUGGUCGUAGGUUCGAAUCCUACCUGGGGAGCCA | |
| 474 | tRNA^{Pro(UGG)}_1 | AGGGAUGUAGCGCAGCUUGGUAGCGCUUUUGUUUUGGGU | 74 |
| | | ACAAAAUGUCACGGGUUCAAAUCCUGUCAUCCCUACCA | |
| 475 | tRNA^{Gln(CUG)} | GGUUCCAUGGUCUAGUGGUCAGGACAUUGGACUCUGAAU | 75 |
| | | CCAGUAACCCGAGUUCAGGUCUC GGUGGAACCUCCA | |
| 476 | tRNA^{Glu(UUG)} | UCCGUUGUCGUCCAGCGGUUAGGAUAUCUGGCUUUCACC | 75 |
| | | CAGGAGACCCGGGUUCGUUUCCCGGCAACGGAACCA | |
| 477 | tRNA^{Cys(GCA)} | GGCUAGGUAACAUAAUGGAAAUGUAUUGGACUGCAAAUCC | 74 |
| | | UGGAAUGACGGUUCGACCCCGUCCUUGGCCUCCA | |
| 478 | tRNA^{Met(CAU)} | AGCGGGGUAGAGUAAUGGUCAACUCAUCAGUCUCAUUAU | 76 |
| | | CUGAAGACUACAGGUUCGAAUCCUGUCCCCGCCUCCA | |
| 479 | tRNA^{Pro(UGG)}_2 | CGAGGUGUAGCGCAGUCUGGUCAGCGCAUCUGUUUUGGG | 78 |
| | | UACAGAGGGCCAUAGGUUCGAAUCCUGUCACCUUGACCA | |
| 480 | tRNA^{Gly(GCC)}_2 | GCACCAGUGGUCUAGUGGUAGAAUAGUACCCUGCCACGG | 74 |
| | | UACAGACCCGGGUUCGUUUCCCGGCUGGUGCACCA | |
| 481 | tRNA^{Asp(GUC)} | GUCGUUGUAGUAUAGUGGUAAGUAUUCCCGCCUGUCACG | 75 |
| | | CGGGUGACCCGGGUUCGAUCCCCGGCAACGGCGCCA | |
| 482 | tRNA^{Try(GCA)} | CCGACCUUAGCUCAGUUGGUAGAGCGGAGGACUGUAGUG | 89 |
| | | UGCUCGUAGCUAUCCUUAGGUCGCUGGUUCGAAUCCGGC | |
| | | UGGUCGGACCA | |
| 483 | tRNA^{Ala(AGC)} | GGGGAUGUAGCUCAGAUGGUAGAGCGCUCGCUUAGCAUG | 76 |
| | | CGAGAGGUACGGGGAUCGAUACCCCGCAUCUCCACCA | |
| 484 | tRNA^{Glu(CUC)} | UCCGUUGUAGUCUAGUUGGUCAGGAUACUCGGCUCUCAC | 76 |
| | | CCGAGAGACCCGGGUUCAAGUCCCGGCAACGGAACCA | |
| 485 | tRNA^{Glu(UUC)}_2 | GUCCCUUUCGUCCAGUGGUUAGGACAUCGUCUUUUCAUG | 75 |
| | | UCGAAGACACGGGUUCGAUUCCCGUAAGGGGUACCA | |
| 486 | tRNA^{Arg(CCU)} | GCGCCUGUAGCUCAGUGGAUAGAGCGUCUGUUUCCUAAG | 76 |
| | | CAGAAAGUCGUAGGUUCGACCCCUACCUGGCGCGCCA | |
| 487 | tRNA^{Val(AAC)} | GGUUUCGUGGUGUAGUUGGUUAUCACGUCAGCCUAACAC | 77 |
| | | ACUGAAGGUCUCCGGUUCGAACCCGGGCGAAGCCACCA | |
| 488 | tRNA^{Val(CAC)} | GUCUGGGUGGUGUAGUCGGUUAUCAUGCUAGUCUCACAC | 77 |
| | | ACUAGAGGUCCCCGGUUCGAACCCGGGCUCAGACACCA | |
| 489 | tRNA^{Ser(UGA)} | GGAUGGAUGUCUGAGCGGUUGGAAGAGUCGGUCUUGAAA | 90 |
| | | ACCGAAGUAUUGAUAGGAAUACCGGGGGUUCGAAUCCCU | |
| | | CUCCAUCCGCCA | |
| 490 | tRNA^{Phe(GAA)}_1 | GCGGGGAUAGCUCAGUUGGGAGAGUGUCAGACUGAAGAU | 76 |
| | | CUAAAGGUCACGUGUUUGAUCCACGUUCACCGCACCA | |
| 491 | tRNA^{His(CAU)} | GCAUCCAUGGCUGAAUGGUUAAAGCGCCCAACUCAUAAUU | 77 |
| | | GGCGAAUUCGUAGGUUCAAUUCCUACUGGAUGCACCA | |
| 492 | tRNA^{Lys(UUU)}_1 | GGGUUGCUAACUCAACGGUAGAGUACUCGGCUUUUAACC | 75 |
| | | GACUAGUUCCGGGUUCGAAUCCCGGGCAACCCACCA | |
| 493 | tRNA^{Ser(UGA)} | GGAGAGAUGGCUGAGUGGUUGAUAGCUCCGGUCUUGAAA | 95 |
| | | ACCGGCAUAGUUUUAACAAAGAACUAUCGAGGGUUCGAAU | |
| | | CCCUCUCUCUCCUCCA | |
| 494 | tRNA^{Ser(GGA)} | AGGAGAGAUGGCCGAGUGGUUGAAGGCGUAGCAUUGGAA | 91 |
| | | CUGCUAUGUAGGCUUUUGUUUACCGAGGGUUCGAAUCCC | |
| | | UCUCUUUCCGCCA | |
| 495 | tRNA^{Gly(UCC)} | GCGGGUAUAGUUUAGUGGUAAAACCCUAGCCUUCCAAGC | 74 |
| | | UAACGAUGCGGGUUCGAUUCCCGCUACCCGCUCCA | |
| 496 | tRNA^{Arg(UCU)} | GCGUCCAUUGUCUAAUGGAUAGGACAGAGGUCUUCUAAA | 75 |
| | | CCUUUGGUAUAGGUUCAAAUCCUAUUGGACGCACCA | |
| 497 | tRNA^{Arg(ACG)} | GGGCCUGUAGCUCAGAGGAUUAGAGCACGUGGCUACGAA | 77 |
| | | CCACGGUGUCGGGGGUUCGAAUCCCUCCUCGCCCACCA | |
| 498 | tRNA^{Cys(GCA)} | GGCGAUAUGGCCGAGUGGUAAGGCGGGGGACUGCAAAUC | 75 |
| | | CUUUUUUCCCCAGUUCAAAUCCGGGUGUCGCCUCCA | |
| 499 | tRNA^{Tyr(GUA)}_1 | GGGUCGAUGCCCGAGCGGUUAAUGGGGACGGACUGUAAA | 87 |
| | | UUCGUUGGCAAUAUGUCUACGCUGGUUCAAAUCCAGCUC | |
| | | GGCCCACCA | |
| 500 | tRNA^{Thr(GGU)} | GCCCUUUUAACUCAGCGGUAGAGUAACGCCAUGGUAAGG | 75 |
| | | CGUAAGUCAUCGGUUCAAAUCCGAUAAGGGGCUCCA | |
| 501 | tRNAT^{hr(UGU)} | GCCUGCUUAGCUCAGAGGUUAGAGCAUCGCAUUUGUAAU | 76 |
| | | GCGAUGGUCAUCGGUUCGAUUCCGAUAGCCGGCUCCA | |
| 502 | tRNA^{Met(CAU)}_2 | ACCUACUUAACUCAGUGGUUAGAGUAUUGCUUUCAUACGG | 76 |
| | | CGGGAGUCAUUGGUUCAAAUCCAAUAGUAGGUACCA | |
| 503 | tRNA^{Leu(UAA)} | GGGGAUAUGGCGGAAUUGGUAGACGCUACGGACUUAAAA | 87 |
| | | UCCGUCGACUUUAAAAUCGUGAGGGUUCAAGUCCCUCUA | |
| | | UCCCCACCA | |
| 504 | tRNA^{Leu(UAG)} | GCCGCUAUGGUGAAAUCGGUAGACACGCUGCUCUUAGGA | 83 |
| | | AGCAGUGCUAGAGCAUCUCGGUUCGAGUCCGAGUGGCGG | |
| | | CACCA | |
| 505 | tRNA^{Phe(GAA)}_2 | GUCGGGAUAGCUCAGCUGGUAGAGCAGAGGACUGAAAAU | 76 |
| | | CCUCGUGUCACCAGUUCAAAUCUGGUUCCUGGCACCA | |
| 506 | tRN^{AVal(UAC)} | AGGGCUAUAGCUCAGUUAGGUAGAGCACCUCGUUUACAC | 77 |
| | | CGAGAAGGUCUACGGUCCGAGUCCGUAUAGCCCUACCA | |
| 507 | tRNA^{Val(GAC)} | AGGGAUAUAACUCAGCGGUAGAGUGUCACCUUGACGUGG | 75 |
| | | UGGAAGUCAUCAGUUCGAGCCUGAUUAUCCCUACCA | |
| 508 | tRNA^{Trp(CCA)} | GCGCUCUUAGUUCAGUUCGGUAGAACGUGGGUCUCCAAA | 77 |
| | | ACCCAAUGUCGUAGGUUCAAAUCCUACAGAGCGUGCCA | |
| 509 | tRNA^{Ile(GAU)} | GGGCUAUUAGCUCAGUGGUAGAGCGCGCCCCUGAUAAGG | 77 |
| | | GCGAGGUCUCUGGUUCAAGUCCAGGAUGGCCCACCA | |
| 510 | tRNA^{Ala(UGC)} | GGGGAUAUAGCUCAGUUGGUAGAGCUCCGCUCUUGCAAG | 76 |
| | | GCGGAUGUCAGCGGUUCGAGUCCGCUUAUCUCCACCA | |
| 511 | tRNA^{Lys(UUU)}_2 | GGGUGUAUAGCUCAGUUGGUAGAGCAUUGGGCUUUUAAC | 76 |
| | | CUAAUGGUCGCAGGUUCAAGUCCUGCUAUACCCACCA | |
| 512 | tRNA^{Lys(CUU)} | CACCCUGUAGCUCAGAGGAAGAGUGGUCGUCUCUUAGCU | 75 |
| | | GACAGGUCGUAGGUUCAAGUCCUACCAGGUUACCCA | |
| 513 | tRNA^{Gln(UUG)}_2 | UGGAGUAUAGCCAAGUGGUAAGGCACCGGUUUUUGGUAC | 67 |
| | | CGAGGUUCGAAUCCUUUUACUCCAGCCA | |
| 514 | tRNA^{Met(CAU)}_3 | GGGCUUAUAGUUUAAUUGGUUGAAACGUACCGCUCAUAAC | 77 |
| | | GGUUAUAUUGUAGGUUCGAGCCCUACUAAGCCUACCA | |
| 515 | tRNA^{Met(CAU)}_4 | GCAUCCAUGGCUGAAUGGUUAAAGCGCCCAACUCAUAAUU | 77 |
| | | GGCGAAUUCGUAGGUUCAAUUCCUACUGGAUGCACCA | |
| 516 | tRNA^{Tyr(GUA)}_2 | GGGAGAGUGGCCGAGUGGUCAAAAGCGACAGACUGUAAA | 86 |
| | | UCUGUUGAAGUUUUUCUACGUAGGUUCGAAUCCUGCCUC | |
| | | UCCCACCA | |
| 517 | tRNA^{Ser(GCU)}_2 | GGAGGUAUGGCUGAGUGGCUUAAGGCAUUGGUUUGCUAA | 91 |
| | | AUCGACAUACAAGAAGAUUGUAUCAUGGGUUCGAAUCCCA | |
| | | UUUCCUCCGCCA | |
| 518 | tRNA^{Phe(GAA)}_3 | GUUCAGGUAGCUCAGCUGGUUAGAGCAAAGGACUGAAAA | 77 |
| | | UCCUUGUGUCAGUGGUUCGAAUCCACUUCUAAGCGCCA | |
| 519 | tRNA^{Phe(AAA)} | GUAACGAUCGAAUAAUGGAAGUUCACGGGGAAAGUCACUA | 78 |
| | | GACCCGAAGCAUUGGUUCAAAUCCAAUUCGUUACUCCA | |
| 520 | tRNA^{Pro(UGG)}_3 | AGGGAUGUAGCGCAGCUUGGUAGCGCCUUUGUUUUGGGU | 82 |
| | | AAAAAAUGUCACGGGUUCCAAUCCAAUCCUGUCAUCCCUA | |
| | | CCA | |
| 521 | tRNA^{Ile(CAU)} | GGGCUAUUAGCUCAGUGGUAGAGCGCGCCCCUGAUAAGG | 75 |
| | | GCGAGGUCUCUGGUUCAAGUCCAGGAUGGCCCACCA | |
| 522 | tRNA^{Gly(GCC)}_3 | GCGGAAAUAGCUUAAUGGUAGAGCAUAGCCUUGCCAAGG | 75 |
| | | CUGAGGUUGAGGGUUCAAGUCCCUCCUUCCGCUCCA | |

The sense sequences of SEQ ID NO: 1 to SEQ ID NO: 232 and the antisense sequences of SEQ ID NO: 233 to SEQ ID NO: 464 as shown in Table 2 are artificially synthesized in accordance with the present invention. In particular, these sequences are derived sequence fragments prepared according to the sequences in Table 1 isolated from Panax ginseng C. A. Mey. The double-stranded RNA molecules are classified into 3 groups: the first group is 5′-terminal group (5′-t) containing a 5′ terminal portion of the corresponding full-length mature tRNA molecules, forming segments of 2-35 nucleotides in length that are cut off in the D-ring, D-arm, anti-codon ring, or anti-codon ring arm. The second group is 3′-terminal group (3′-t) containing a 3′ terminal portion with CCA tail of the corresponding full-length mature tRNA molecules, forming segments of 2-35 nucleotides in length that are cut off in the T-ring, T-arm, anti-codon ring, or anti-codon ring arm. The third group is anticodon group RNA molecules containing the anticodon loop portion of the corresponding full-length mature tRNA molecules, forming segments of 2-24 nucleotides in length that are cut off in anti-codon ring, or anti-codon ring arm. In the embodiment, tRFs derived from tRNA^{His(GUG)} comprises 5′-tRFs “GCGGAUGUAGCCAAGUGGAUCA” that belongs to the family of 5′-tRFs with a length of 22 mer, 3′-tRFs “UCAAUUCCCGUCGUUCGCCCCA” that belongs to the family of 3′-tRFs with a length of 22 mer, 5′-tRFs “GCGGAUGUAGCCAAGUGGA” that belongs to the family of 5′-tRFs with a length of 19 mer, and 3′-tRFs “AUUCCCGUCGUUCGCCCCA” that belongs to the family of 3′-tRFs with a length of 19 mer, and anti-codon-tRFs “GUGGAUUGUGAAUCCAC” belongs to the family of anti-codon-tRFs with length of 17 mer.

Each of the sense sequences together with the corresponding antisense sequence form a double-stranded RNA molecule. As shown in Table 2, the sense sequence of SEQ ID NO: 1 and the antisense sequence of SEQ ID NO: 233 form a double-stranded RNA molecule with a length of 22 base pairs, and the resultant RNA molecule is denoted as HC70 for easy reference. Similarly, the sense sequence of SEQ ID NO: 2 and the antisense sequence of SEQ ID NO: 234 form a double-stranded RNA molecule with a length of 19 base pairs, and the resultant RNA molecule is denoted as HC50. Other RNA molecules of the present invention are presented in the Table 2.

The double-stranded RNA molecules are classified into 2 groups, namely a 5′-terminal group (5′-t), and a 3′-terminal group (3′-t). The 5′-t group RNA molecules contain a 5′ terminal portion of the corresponding full-length RNA molecules isolated from the plant; and the 3′-t group RNA molecules contain a 3′ terminal portion of the corresponding full-length RNA molecules isolated from the plant.

In another embodiment, RNA molecules may contain the anticodon loop portion of the corresponding full-length RNA molecules isolated from the plant and referred as anticodon group RNA molecules. The sense sequences of SEQ ID NO: 1 to SEQ ID NO: 232 can be generated by cleavage at different sites on the full-length RNA molecules SEQ ID NO: 465 to 522.

In addition, the RNA molecule of the present invention may comprise a 3′ overhang, preferably comprise 2 mer of 3′ overhangs. The provision of the 3′ overhang improves the stability of the RNA molecules.

TABLE 2
RNA molecules derived from the sequences in Table 1 through artificial
synthesis according to the present invention.

| | | SEQ | | SEQ | | | |
|---|---|---|---|---|---|---|---|
| | | ID | Sense sequence | ID | Antisense sequence | Length | |
| Source | Code | NO. | (5′ to 3′) | NO. | (5′ to 3′) | (bp) | Group |
| tRNA^{His(GUG)} | HC70 | 1 | GCGGAUGUAGCC | 233 | UGAUCCACUUGGC | 22 | 5′-t |
| | | | AAGUGGAUCA | | UACAUCCGC | | |
| | HC50 | 2 | GCGGAUGUAGCC | 234 | UCCACUUGGCUAC | 19 | |
| | | | AAGUGGA | | AUCCGC | | |
| | HC71 | 3 | UCAAUUCCCGUC | 235 | UGGGGCGAACGA | 22 | 3'-t |
| | | | GUUCGCCCCA | | CGGGAAUUGA | | |
| | HC51 | 4 | AUUCCCGUCGUU | 236 | UGGGGCGAACGA | 19 | |
| | | | CGCCCCA | | CGGGAAU | | |
| tRNA^{Asp(GUC)} | HC72 | 5 | GGGAUUGUAGUU | 237 | UGACCGAUUGAAC | 22 | 5'-t |
| | | | CAAUCGGUCA | | UACAAUCCC | | |
| | HC52 | 6 | GGGAUUGUAGUU | 238 | CCGAUUGAACUAC | 19 | |
| | | | CAAUCGG | | AAUCCC | | |
| | HC73 | 7 | UCGAGCCCCGUC | 239 | UGGCGGGACUGA | 22 | 3'-t |
| | | | AGUCCCGCCA | | CGGGGCUCGA | | |
| | HC53 | 8 | AGCCCCGUCAGU | 240 | UGGCGGGACUGA | 19 | |
| | | | CCCGCCA | | CGGGGCU | | |
| tRNA^{Gly(GCC)}_1 | HC74 | 9 | GCGGAUAUAGUC | 241 | UUUACCAUUCGAC | 22 | 5'-t |
| | | | GAAUGGUAAA | | UAUAUCCGC | | |
| | HC54 | 10 | GCGGAUAUAGUC | 242 | ACCAUUCGACUAU | 19 | |
| | | | GAAUGGU | | AUCCGC | | |
| | HC75 | 11 | UCGAUUCCCGCU | 243 | UGGGGCGGAUAG | 22 | 3'-t |
| | | | AUCCGCCCCA | | CGGGAAUCGA | | |
| | HC55 | 12 | AUUCCCGCUAUC | 244 | UGGGGCGGAUAG | 19 | |
| | | | CGCCCCA | | CGGGAAU | | |
| tRNA^{Leu(CAA)} | HC76 | 13 | GCCUUGGUGGUG | 245 | UCUACCAUUUCAC | 22 | 5'-t |
| | | | AAAUGGUAGA | | CACCAAGGC | | |
| | HC56 | 14 | GCCUUGGUGGUG | 246 | ACCAUUUCACCAC | 19 | |
| | | | AAAUGGU | | CAAGGC | | |
| | HC77 | 15 | UCGAGUCCUCUU | 247 | UGGUGCCUUGAA | 22 | 3'-t |
| | | | CAAGGCACCA | | GAGGACUCGA | | |
| | HC57 | 16 | AGUCCUCUUCAA | 248 | UGGUGCCUUGAA | 19 | |
| | | | GGCACCA | | GAGGACU | | |
| tRNA^{Met(CAU)}_1 | HC78 | 17 | CGCGGAGUAGAG | 249 | UACCAAACUGCUC | 22 | 5'-t |
| | | | CAGUUUGGUA | | UACUCCGCG | | |
| | HC58 | 18 | CGCGGAGUAGAG | 250 | CAAACUGCUCUAC | 19 | |
| | | | CAGUUUG | | UCCGCG | | |
| | HC79 | 19 | UCAAAUCCUGUC | 251 | UGGUUGCGGAGA | 22 | 3'-t |
| | | | UCCGCAACCA | | CAGGAUUUGA | | |
| | HC59 | 20 | AAUCCUGUCUCC | 252 | UGGUUGCGGAGA | 19 | |
| | | | GCAACCA | | CAGGAUU | | |
| tRNA^{Ser(GCU)}_1 | HC80 | 21 | GGAGAGAUGGCU | 253 | UAGUCCACUCAGC | 22 | 5'-t |
| | | | GAGUGGACUA | | CAUCUCUCC | | |
| | HC60 | 22 | GGAGAGAUGGCU | 254 | UCCACUCAGCCAU | 19 | |
| | | | GAGUGGA | | CUCUCC | | |
| | HC81 | 23 | GGAGAGAUGGCU | 255 | UGGCGGAAAGAG | 22 | 3'-t |
| | | | GAGUGGACUA | | AGGGAUUCGA | | |
| | HC61 | 24 | AAUCCCUCUCUU | 256 | UGGCGGAAAGAG | 19 | |
| | | | UCCGCCA | | AGGGAUU | | |
| tRNA^{Gln(UUG)}_1 | HC82 | 25 | UGGGGCGUGGC | 257 | CUUACCACUUGG | 22 | 5'-t |
| | | | CAAGUGGUAAG | | CCACGCCCCA | | |
| | HC62 | 26 | UGGGGCGUGGC | 258 | ACCACUUGGCCAC | 19 | |
| | | | CAAGUGGU | | GCCCCA | | |
| | HC83 | 27 | UCGAAUCCUUCC | 259 | UGGCUGGGACGG | 22 | 3'-t |
| | | | GUCCCAGCCA | | AAGGAUUCGA | | |
| | HC63 | 28 | AAUCCUUCCGUC | 260 | UGGCUGGGACGG | 19 | |
| | | | CCAGCCA | | AAGGAUU | | |
| tRNA^{Glu(UUC)}_1 | HC84 | 29 | GCCCCCAUCGUC | 261 | UGAACCACUAGAC | 22 | 5'-t |
| | | | UAGUGGUUCA | | GAUGGGGGC | | |
| | HC64 | 30 | GCCCCCAUCGUC | 262 | ACCACUAGACGAU | 19 | |
| | | | UAGUGGU | | GGGGGC | | |
| | HC85 | 31 | UCGACUUCCCCU | 263 | UGGUACCCCCAG | 22 | 3'-t |
| | | | GGGGGUACCA | | GGGAAGUCGA | | |
| | HC65 | 32 | ACUUCCCCUGGG | 264 | UGGUACCCCCAG | 19 | |
| | | | GGUACCA | | GGGAAGU | | |
| tRNA^{Asn(GUU)} | HC86 | 33 | UCCUCAGUAGCU | 265 | UCUACCACUGAGC | 22 | 5'-t |
| | | | CAGUGGUAGA | | UACUGAGGA | | |
| | HC66 | 34 | UCCUCAGUAGCU | 266 | ACCACUGAGCUAC | 19 | |
| | | | CAGUGGU | | UGAGGA | | |
| | HC87 | 35 | UCGAAUCCUACC | 267 | UGGCUCCCCAGG | 22 | 3'-t |
| | | | UGGGGAGCCA | | UAGGAUUCGA | | |
| | HC67 | 36 | AAUCCUACCUGG | 268 | UGGCUCCCCAGG | 19 | |
| | | | GGAGCCA | | UAGGAUU | | |
| tRNA^{Pro(UGG)}_1 | HC88 | 37 | AGGGAUGUAGCG | 269 | UACCAAGCUGCG | 22 | 5'-t |
| | | | CAGCUUGGUA | | CUACAUCCCU | | |
| | HC68 | 38 | AGGGAUGUAGCG | 270 | CAAGCUGCGCUA | 19 | |
| | | | CAGCUUG | | CAUCCCU | | |
| | HC89 | 39 | GGUUCAAAUCCU | 271 | UAGGGAUGACAG | 22 | 3'-t |
| | | | GUCAUCCCUA | | GAUUUGAACC | | |
| | HC69 | 40 | UCAAAUCCUGUC | 272 | UAGGGAUGACAG | 19 | |
| | | | AUCCCUA | | GAUUUGA | | |
| tRNA^{Gln(CUG)} | HC90 | 41 | GGUUCCAUGGUC | 273 | CUGACCACUAGAC | 22 | 5'-t |
| | | | UAGUGGUCAG | | CAUGGAACC | | |
| | HC91 | 42 | GGUUCCAUGGUC | 274 | ACCACUAGACCAU | 19 | |
| | | | UAGUGGU | | GGAACC | | |
| | HC92 | 43 | UCAGGUCUCGGU | 275 | UGGAGGUUCCAC | 22 | 3'-t |
| | | | GGAACCUCCA | | CGAGACCUGA | | |
| | HC93 | 44 | GGUCUCGGUGGA | 276 | UGGAGGUUCCAC | 19 | |
| | | | ACCUCCA | | CGAGACC | | |
| tRNA^{Glu(UUG)} | HC94 | 45 | UCCGUUGUCGUC | 277 | CUAACCGCUGGA | 22 | 5'-t |
| | | | CAGCGGUUAG | | CGACAACGGA | | |
| | HC95 | 46 | UCCGUUGUCGUC | 278 | ACCGCUGGACGA | 19 | |
| | | | CAGCGGU | | CAACGGA | | |
| | HC96 | 47 | UCGUUUCCCGGC | 279 | UGGUUCCGUUGC | 22 | 3'-t |
| | | | AACGGAACCA | | CGGGAAACGA | | |
| | HC97 | 48 | UUUCCCGGCAAC | 280 | UGGUUCCGUUGC | 19 | |
| | | | GGAACCA | | CGGGAAA | | |
| tRNA^{Cys(GCA)} | HC98 | 49 | GGCUAGGUAACA | 281 | AUUUCCAUUAUGU | 22 | 5'-t |
| | | | UAAUGGAAAU | | UACCUAGCC | | |
| | HC99 | 50 | GGCUAGGUAACA | 282 | UCCAUUAUGUUAC | 19 | |
| | | | UAAUGGA | | CUAGCC | | |
| | HC100 | 51 | UCGACCCCGUCC | 283 | UGGAGGCCAAGG | 22 | 3'-t |
| | | | UUGGCCUCCA | | ACGGGGUCGA | | |
| | HC101 | 52 | ACCCCGUCCUUG | 284 | UGGAGGCCAAGG | 19 | |
| | | | GCCUCCA | | ACGGGGU | | |
| tRNA^{Met(CAU)}_1 | HC102 | 53 | AGCGGGGUAGAG | 285 | UUGACCAUUACUC | 22 | 5'-t |
| | | | UAAUGGUCAA | | UACCCCGCU | | |
| | HC103 | 54 | AGCGGGGUAGAG | 286 | ACCAUUACUCUAC | 19 | |
| | | | UAAUGGU | | CCCGCU | | |
| | HC104 | 55 | UCGAAUCCUGUC | 287 | UGGAGGCGGGGA | 22 | 3'-t |
| | | | CCCGCCUCCA | | CAGGAUUCGA | | |
| | HC105 | 56 | AAUCCUGUCCCC | 288 | UGGAGGCGGGGA | 19 | |
| | | | GCCUCCA | | CAGGAUU | | |
| tRNA^{Pro(UGG)}_2 | HC106 | 57 | CGAGGUGUAGCG | 289 | GACCAGACUGCG | 22 | 5'-t |
| | | | CAGUCUGGUC | | CUACACCUCG | | |
| | HC107 | 58 | CGAGGUGUAGCG | 290 | CAGACUGCGCUA | 19 | |
| | | | CAGUCUG | | CACCUCG | | |
| | HC108 | 59 | UCGAAUCCUGUC | 291 | UGGUCAAGGUGA | 22 | 3'-t |
| | | | ACCUUGACCA | | CAGGAUUCGA | | |
| | HC109 | 60 | AAUCCUGUCACC | 292 | UGGUCAAGGUGA | 19 | |
| | | | UUGACCA | | CAGGAUU | | |
| tRNA^{Gly(GCC)}_2 | HC110 | 61 | GCACCAGUGGUC | 293 | UCUACCACUAGAC | 22 | 5'-t |
| | | | UAGUGGUAGA | | CACUGGUGC | | |
| | HC111 | 62 | GCACCAGUGGUC | 294 | ACCACUAGACCAC | 19 | |
| | | | UAGUGGU | | UGGUGC | | |
| | HC112 | 63 | UCGUUUCCCGGC | 295 | UGGUGCACCAGC | 22 | 3'-t |
| | | | UGGUGCACCA | | CGGGAAACGA | | |
| | HC113 | 64 | UUUCCCGGCUGG | 296 | UGGUGCACCAGC | 19 | |
| | | | UGCACCA | | CGGGAAA | | |
| tRNA^{Asp(GUC)} | HC114 | 65 | GUCGUUGUAGUA | 297 | CUUACCACUAUAC | 22 | 5'-t |
| | | | UAGUGGUAAG | | UACAACGAC | | |
| | HC115 | 66 | GUCGUUGUAGUA | 298 | ACCACUAUACUAC | 19 | |
| | | | UAGUGGU | | AACGAC | | |
| | HC116 | 67 | UCGAUCCCCGGC | 299 | UGGCGCCGUUGC | 22 | 3'-t |
| | | | AACGGCGCCA | | CGGGGAUCGA | | |
| | HC117 | 68 | AUCCCCGGCAAC | 300 | UGGCGCCGUUGC | 19 | |
| | | | GGCGCCA | | CGGGGAU | | |
| tRNA^{Try(GCA)} | HC118 | 69 | CCGACCUUAGCU | 301 | CUACCAACUGAGC | 22 | 5'-t |
| | | | CAGUUGGUAG | | UAAGGUCGG | | |
| | HC119 | 70 | CCGACCUUAGCU | 302 | CCAACUGAGCUAA | 19 | |
| | | | CAGUUGG | | GGUCGG | | |
| | HC120 | 71 | UCGAAUCCGGCU | 303 | UGGUCCGACCAG | 22 | 3'-t |
| | | | GGUCGGACCA | | CCGGAUUCGA | | |
| | HC121 | 72 | AAUCCGGCUGGU | 304 | UGGUCCGACCAG | 19 | |
| | | | CGGACCA | | CCGGAUU | | |
| tRNA^{Ala(AGC)} | HC122 | 73 | GGGGAUGUAGCU | 305 | CUACCAUCUGAGC | 22 | 5'-t |
| | | | CAGAUGGUAG | | UACAUCCCC | | |
| | HC123 | 74 | GGGGAUGUAGCU | 306 | CCAUCUGAGCUAC | 19 | |
| | | | CAGAUGG | | AUCCCC | | |
| | HC124 | 75 | UCGAUACCCCGC | 307 | UGGUGGAGAUGC | 22 | 3'-t |
| | | | AUCUCCACCA | | GGGGUAUCGA | | |
| | HC125 | 76 | AUACCCCGCAUC | 308 | UGGUGGAGAUGC | 19 | |
| | | | UCCACCA | | GGGGUAU | | |
| tRNA^{Glu(CUC)} | HC126 | 77 | UCCGUUGUAGUC | 309 | UGACCAACUAGAC | 22 | 5'-t |
| | | | UAGUUGGUCA | | UACAACGGA | | |
| | HC127 | 78 | UCCGUUGUAGUC | 310 | CCAACUAGACUAC | 19 | |
| | | | UAGUUGG | | AACGGA | | |
| | HC128 | 79 | UCAAGUCCCGGC | 311 | UGGUUCCGUUGC | 22 | 3'-t |
| | | | AACGGAACCA | | CGGGACUUGA | | |
| | HC129 | 80 | AGUCCCGGCAAC | 312 | UGGUUCCGUUGC | 19 | |
| | | | GGAACCA | | CGGGACU | | |
| tRNA^{Glu(UUC)}_2 | HC130 | 81 | GUCCCUUUCGUC | 313 | CUAACCACUGGAC | 22 | 5'-t |
| | | | CAGUGGUUAG | | GAAAGGGAC | | |
| | HC131 | 82 | GUCCCUUUCGUC | 314 | ACCACUGGACGAA | 19 | |
| | | | CAGUGGU | | AGGGAC | | |
| | HC132 | 83 | UCGAUUCCCGUA | 315 | UGGUACCCCUUA | 22 | 3'-t |
| | | | AGGGGUACCA | | CGGGAAUCGA | | |
| | HC133 | 84 | AUUCCCGUAAGG | 316 | UGGUACCCCUUA | 19 | |
| | | | GGUACCA | | CGGGAAU | | |
| tRNA^{Arg(CCU)} | HC134 | 85 | GCGCCUGUAGCU | 317 | CUAUCCACUGAGC | 22 | 5'-t |
| | | | CAGUGGAUAG | | UACAGGCGC | | |
| | HC135 | 86 | GCGCCUGUAGCU | 318 | UCCACUGAGCUAC | 19 | |
| | | | CAGUGGA | | AGGCGC | | |
| | HC136 | 87 | UCGACCCCUACC | 319 | UGGCGCGCCAGG | 22 | 3'-t |
| | | | UGGCGCGCCA | | UAGGGGUCGA | | |
| | HC137 | 88 | ACCCCUACCUGG | 320 | UGGCGCGCCAGG | 19 | |
| | | | CGCGCCA | | UAGGGGU | | |
| tRNA^{Val(AAC)} | HC138 | 89 | GGUUUCGUGGUG | 321 | UAACCAACUACAC | 22 | 5'-t |
| | | | UAGUUGGUUA | | CACGAAACC | | |
| | HC139 | 90 | GGUUUCGUGGUG | 322 | CCAACUACACCAC | 19 | |
| | | | UAGUUGG | | GAAACC | | |
| | HC140 | 91 | UCGAACCCGGGC | 323 | UGGUGGCUUCGC | 22 | 3'-t |
| | | | GAAGCCACCA | | CCGGGUUCGA | | |
| | HC141 | 92 | AACCCGGGCGAA | 324 | UGGUGGCUUCGC | 19 | |
| | | | GCCACCA | | CCGGGUU | | |
| tRNA^{Val(CAC)} | HC142 | 93 | GUCUGGGUGGU | 325 | UAACCGACUACAC | 22 | 5'-t |
| | | | GUAGUCGGUUA | | CACCCAGAC | | |
| | HC143 | 94 | GUCUGGGUGGU | 326 | CCGACUACACCAC | 19 | |
| | | | GUAGUCGG | | CCAGAC | | |
| | HC144 | 95 | UCGAACCCGGGC | 327 | UGGUGUCUGAGC | 22 | 3'-t |
| | | | UCAGACACCA | | CCGGGUUCGA | | |
| | HC145 | 96 | AACCCGGGCUCA | 328 | UGGUGUCUGAGC | 19 | |
| | | | GACACCA | | CCGGGUU | | |
| tRNA^{Ser(UGA)} | HC146 | 97 | GGAUGGAUGUCU | 329 | CCAACCGCUCAGA | 22 | 5'-t |
| | | | GAGCGGUUGG | | CAUCCAUCC | | |
| | HC147 | 98 | GGAUGGAUGUCU | 330 | ACCGCUCAGACAU | 19 | |
| | | | GAGCGGU | | CCAUCC | | |
| | HC148 | 99 | UCGAAUCCCUCU | 331 | UGGCGGAUGGAG | 22 | 3'-t |
| | | | CCAUCCGCCA | | AGGGAUUCGA | | |
| | HC149 | 100 | AAUCCCUCUCCA | 332 | UGGCGGAUGGAG | 19 | |
| | | | UCCGCCA | | AGGGAUU | | |
| tRNA^{Phe(GAA)} | HC150 | 101 | GCGGGGAUAGCU | 333 | CUCCCAACUGAGC | 22 | 5'-t |
| | | | CAGUUGGGAG | | UAUCCCCGC | | |
| | HC151 | 102 | GCGGGGAUAGCU | 334 | CCAACUGAGCUAU | 19 | |
| | | | CAGUUGG | | CCCCGC | | |
| | HC152 | 103 | UUGAUCCACGUU | 335 | UGGUGCGGUGAA | 22 | 3'-t |
| | | | CACCGCACCA | | CGUGGAUCAA | | |
| | HC153 | 104 | AUCCACGUUCAC | 336 | UGGUGCGGUGAA | 19 | |
| | | | CGCACCA | | CGUGGAU | | |
| tRNA^{His(CAU)} | HC154 | 105 | GCAUCCAUGGCU | 337 | UUAACCAUUCAGC | 22 | 5'-t |
| | | | GAAUGGUUAA | | CAUGGAUGC | | |
| | HC155 | 106 | GCAUCCAUGGCU | 338 | ACCAUUCAGCCAU | 19 | |
| | | | GAAUGGU | | GGAUGC | | |
| | HC156 | 107 | UCAAUUCCUACU | 339 | UGGUGCAUCCAG | 22 | 3'-t |
| | | | GGAUGCACCA | | UAGGAAUUGA | | |
| | HC157 | 108 | AUUCCUACUGGA | 340 | UGGUGCAUCCAG | 19 | |
| | | | UGCACCA | | UAGGAAU | | |
| tRNA^{Lys(UUU)}_1 | HC158 | 109 | GGGUUGCUAACU | 341 | UCUACCGUUGAG | 22 | 5'-t |
| | | | CAACGGUAGA | | UUAGCAACCC | | |
| | HC159 | 110 | GGGUUGCUAACU | 342 | ACCGUUGAGUUA | 19 | |
| | | | CAACGGU | | GCAACCC | | |
| | HC160 | 111 | UCGAAUCCCGGG | 343 | UGGUGGGUUGCC | 22 | 3'-t |
| | | | CAACCCACCA | | CGGGAUUCGA | | |
| | HC161 | 112 | AAUCCCGGGCAA | 344 | UGGUGGGUUGCC | 19 | |
| | | | CCCACCA | | CGGGAUU | | |
| tRNA^{Ser(UGA)} | HC162 | 113 | GGAGAGAUGGCU | 345 | UCAACCACUCAGC | 22 | 5'-t |
| | | | GAGUGGUUGA | | CAUCUCUCC | | |
| | HC163 | 114 | GGAGAGAUGGCU | 346 | ACCACUCAGCCAU | 19 | |
| | | | GAGUGGU | | CUCUCC | | |
| | HC164 | 115 | UCGAAUCCCUCU | 347 | UGGAGGAGAGAG | 22 | 3'-t |
| | | | CUCUCCUCCA | | AGGGAUUCGA | | |
| | HC165 | 116 | AAUCCCUCUCUC | 348 | UGGAGGAGAGAG | 19 | |
| | | | UCCUCCA | | AGGGAUU | | |
| tRNA^{Ser(GGA)} | HC166 | 117 | AGGAGAGAUGGC | 349 | CAACCACUCGGCC | 22 | 5'-t |
| | | | CGAGUGGUUG | | AUCUCUCCU | | |
| | HC167 | 118 | AGGAGAGAUGGC | 350 | CCACUCGGCCAU | 19 | |
| | | | CGAGUGG | | CUCUCCU | | |
| | HC168 | 119 | UCGAAUCCCUCU | 351 | UGGCGGAAAGAG | 22 | 3'-t |
| | | | CUUUCCGCCA | | AGGGAUUCGA | | |
| | HC169 | 120 | AAUCCCUCUCUU | 352 | UGGCGGAAAGAG | 19 | |
| | | | UCCGCCA | | AGGGAUU | | |
| tRNA^{Gly(UCC)} | HC170 | 121 | GCGGGUAUAGUU | 353 | UUUACCACUAAAC | 22 | 5'-t |
| | | | UAGUGGUAAA | | UAUACCCGC | | |
| | HC171 | 122 | GCGGGUAUAGUU | 354 | ACCACUAAACUAU | 19 | |
| | | | UAGUGGU | | ACCCGC | | |
| | HC172 | 123 | UCGAUUCCCGCU | 355 | UGGAGCGGGUAG | 22 | 3'-t |
| | | | ACCCGCUCCA | | CGGGAAUCGA | | |
| | HC173 | 124 | AUUCCCGCUACC | 356 | UGGAGCGGGUAG | 19 | |
| | | | CGCUCCA | | CGGGAAU | | |
| tRNA^{Arg(UCU)} | HC174 | 125 | GCGUCCAUUGUC | 357 | CUAUCCAUUAGAC | 22 | 5'-t |
| | | | UAAUGGAUAG | | AAUGGACGC | | |
| | HC175 | 126 | GCGUCCAUUGUC | 358 | UCCAUUAGACAAU | 19 | |
| | | | UAAUGGA | | GGACGC | | |
| | HC176 | 127 | UCAAAUCCUAUU | 359 | UGGUGCGUCCAA | 22 | 3'-t |
| | | | GGACGCACCA | | UAGGAUUUGA | | |
| | HC177 | 128 | AAUCCUAUUGGA | 360 | UGGUGCGUCCAA | 19 | |
| | | | CGCACCA | | UAGGAUU | | |
| tRNA^{Arg(ACG)} | HC178 | 129 | GGGCCUGUAGCU | 361 | UAAUCCUCUGAGC | 22 | 5'-t |
| | | | CAGAGGAUUA | | UACAGGCCC | | |
| | HC179 | 130 | GGGCCUGUAGCU | 362 | UCCUCUGAGCUA | 19 | |
| | | | CAGAGGA | | CAGGCCC | | |
| | HC180 | 131 | UCGAAUCCCUCC | 363 | UGGUGGGCGAGG | 22 | 3'-t |
| | | | UCGCCCACCA | | AGGGAUUCGA | | |
| | HC181 | 132 | AAUCCCUCCUCG | 364 | UGGUGGGCGAGG | 19 | |
| | | | CCCACCA | | AGGGAUU | | |
| tRNA^{Cys(GCA)} | HC182 | 133 | GGCGAUAUGGCC | 365 | CUUACCACUCGG | 22 | 5'-t |
| | | | GAGUGGUAAG | | CCAUAUCGCC | | |
| | HC183 | 134 | GGCGAUAUGGCC | 366 | ACCACUCGGCCAU | 19 | |
| | | | GAGUGGU | | AUCGCC | | |
| | HC184 | 135 | UCAAAUCCGGGU | 367 | UGGAGGCGACAC | 22 | 3'-t |
| | | | GUCGCCUCCA | | CCGGAUUUGA | | |
| | HC185 | 136 | AAUCCGGGUGUC | 368 | UGGAGGCGACAC | 19 | |
| | | | GCCUCCA | | CCGGAUU | | |
| tRNA^{Tyr(GUA)}_1 | HC186 | 137 | GGGUCGAUGCCC | 369 | UUAACCGCUCGG | 22 | 5'-t |
| | | | GAGCGGUUAA | | GCAUCGACCC | | |
| | HC187 | 138 | GGGUCGAUGCCC | 370 | ACCGCUCGGGCA | 19 | |
| | | | GAGCGGU | | UCGACCC | | |
| | HC188 | 139 | UCAAAUCCAGCU | 371 | UGGUGGGCCGAG | 22 | 3'-t |
| | | | CGGCCCACCA | | CUGGAUUUGA | | |
| | HC189 | 140 | AAUCCAGCUCGG | 372 | UGGUGGGCCGAG | 19 | |
| | | | CCCACCA | | CUGGAUU | | |
| tRNA^{Thr(GGU)} | HC190 | 141 | GCCCUUUUAACU | 373 | UCUACCGCUGAG | 22 | 5'-t |
| | | | CAGCGGUAGA | | UUAAAAGGGC | | |
| | HC191 | 142 | GCCCUUUUAACU | 374 | ACCGCUGAGUUAA | 19 | |
| | | | CAGCGGU | | AAGGGC | | |
| | HC192 | 143 | UCAAAUCCGAUA | 375 | UGGAGCCCCUUA | 22 | 3'-t |
| | | | AGGGGCUCCA | | UCGGAUUUGA | | |
| | HC193 | 144 | AAUCCGAUAAGG | 376 | UGGAGCCCCUUA | 19 | |
| | | | GGCUCCA | | UCGGAUU | | |
| tRNA^{Thr(UGU)} | HC194 | 145 | GCCUGCUUAGCU | 377 | CUAACCUCUGAGC | 22 | 5'-t |
| | | | CAGAGGUUAG | | UAAGCAGGC | | |
| | HC195 | 146 | GCCUGCUUAGCU | 378 | ACCUCUGAGCUAA | 19 | |
| | | | CAGAGGU | | GCAGGC | | |
| | HC196 | 147 | UCGAUUCCGAUA | 379 | UGGAGCCGGCUA | 22 | 3'-t |
| | | | GCCGGCUCCA | | UCGGAAUCGA | | |
| | HC197 | 148 | AUUCCGAUAGCC | 380 | UGGAGCCGGCUA | 19 | |
| | | | GGCUCCA | | UCGGAAU | | |
| tRNA^{Met(CAU)}_2 | HC198 | 149 | ACCUACUUAACU | 381 | CUAACCACUGAGU | 22 | 5'-t |
| | | | CAGUGGUUAG | | UAAGUAGGU | | |
| | HC199 | 150 | ACCUACUUAACU | 382 | ACCACUGAGUUAA | 19 | |
| | | | CAGUGGU | | GUAGGU | | |
| | HC200 | 151 | UCAAAUCCAAUA | 383 | UGGUACCUACUAU | 22 | 3'-t |
| | | | GUAGGUACCA | | UGGAUUUGA | | |
| | HC201 | 152 | AAUCCAAUAGUA | 384 | UGGUACCUACUAU | 19 | |
| | | | GGUACCA | | UGGAUU | | |
| tRNA^{Leu(UAA)} | HC202 | 153 | GGGGAUAUGGCG | 385 | CUACCAAUUCCGC | 22 | 5'-t |
| | | | GAAUUGGUAG | | CAUAUCCCC | | |
| | HC203 | 154 | GGGGAUAUGGCG | 386 | CCAAUUCCGCCAU | 19 | |
| | | | GAAUUGG | | AUCCCC | | |
| | HC204 | 155 | UCAAGUCCCUCU | 387 | UGGUGGGGAUAG | 22 | 3'-t |
| | | | AUCCCCACCA | | AGGGACUUGA | | |
| | HC205 | 156 | AGUCCCUCUAUC | 388 | UGGUGGGGAUAG | 19 | |
| | | | CCCACCA | | AGGGACU | | |
| tRNA^{Leu(UAG)} | HC206 | 157 | GCCGCUAUGGUG | 389 | CUACCGAUUUCAC | 22 | 5'-t |
| | | | AAAUCGGUAG | | CAUAGCGGC | | |
| | HC207 | 158 | GCCGCUAUGGUG | 390 | CCGAUUUCACCAU | 19 | |
| | | | AAAUCGG | | AGCGGC | | |
| | HC208 | 159 | UCGAGUCCGAGU | 391 | UGGUGCCGCCAC | 22 | 3'-t |
| | | | GGCGGCACCA | | UCGGACUCGA | | |
| | HC209 | 160 | AGUCCGAGUGGC | 392 | UGGUGCCGCCAC | 19 | |
| | | | GGCACCA | | UCGGACU | | |
| tRNA^{Phe(GAA)}_2 | HC210 | 161 | GUCGGGAUAGCU | 393 | CUACCAGCUGAG | 22 | 5'-t |
| | | | CAGCUGGUAG | | CUAUCCCGAC | | |
| | HC211 | 162 | GUCGGGAUAGCU | 394 | CCAGCUGAGCUA | 19 | |
| | | | CAGCUGG | | UCCCGAC | | |
| | HC212 | 163 | UCAAAUCUGGUU | 395 | UGGUGCCAGGAA | 22 | 3'-t |
| | | | CCUGGCACCA | | CCAGAUUUGA | | |
| | HC213 | 164 | AAUCUGGUUCCU | 396 | UGGUGCCAGGAA | 19 | |
| | | | GGCACCA | | CCAGAUU | | |
| tRNA^{Val(UAC)} | HC214 | 165 | AGGGCUAUAGCU | 397 | UACCUAACUGAGC | 22 | 5'-t |
| | | | CAGUUAGGUA | | UAUAGCCCU | | |
| | HC215 | 166 | AGGGCUAUAGCU | 398 | CUAACUGAGCUAU | 19 | |
| | | | CAGUUAG | | AGCCCU | | |
| | HC216 | 167 | CCGAGUCCGUAU | 399 | UGGUAGGGCUAU | 22 | 3'-t |
| | | | AGCCCUACCA | | ACGGACUCGG | | |
| | HC217 | 168 | AGUCCGUAUAGC | 400 | UGGUAGGGCUAU | 19 | |
| | | | CCUACCA | | ACGGACU | | |
| tRNA^{Val(GAC)} | HC218 | 169 | AGGGAUAUAACU | 401 | UCUACCGCUGAG | 22 | 5'-t |
| | | | CAGCGGUAGA | | UUAUAUCCCU | | |
| | HC219 | 170 | AGGGAUAUAACU | 402 | ACCGCUGAGUUA | 19 | |
| | | | CAGCGGU | | UAUCCCU | | |
| | HC220 | 171 | UCGAGCCUGAUU | 403 | UGGUAGGGAUAA | 22 | 3'-t |
| | | | AUCCCUACCA | | UCAGGCUCGA | | |
| | HC221 | 172 | AGCCUGAUUAUC | 404 | UGGUAGGGAUAA | 19 | |
| | | | CCUACCA | | UCAGGCU | | |
| tRNA^{Trp(CCA)} | HC222 | 173 | GCGCUCUUAGUU | 405 | UACCGAACUGAAC | 22 | 5'-t |
| | | | CAGUUCGGUA | | UAAGAGCGC | | |
| | HC223 | 174 | GCGCUCUUAGUU | 406 | CGAACUGAACUAA | 19 | |
| | | | CAGUUCG | | GAGCGC | | |
| | HC224 | 175 | UCAAAUCCUACA | 407 | UGGCACGCUCUG | 22 | 3'-t |
| | | | GAGCGUGCCA | | UAGGAUUUGA | | |
| | HC225 | 176 | AAUCCUACAGAG | 408 | UGGCACGCUCUG | 19 | |
| | | | CGUGCCA | | UAGGAUU | | |
| tRNA^{Ile(GAU)} | HC226 | 177 | GGGCUAUUAGCU | 409 | UCUACCACUGAGC | 22 | 5'-t |
| | | | CAGUGGUAGA | | UAAUAGCCC | | |
| | HC227 | 178 | GGGCUAUUAGCU | 410 | ACCACUGAGCUAA | 19 | |
| | | | CAGUGGU | | UAGCCC | | |
| | HC228 | 179 | UCAAGUCCAGGA | 411 | UGGUGGGCCAUC | 22 | 3'-t |
| | | | UGGCCCACCA | | CUGGACUUGA | | |
| | HC229 | 180 | AGUCCAGGAUGG | 412 | UGGUGGGCCAUC | 19 | |
| | | | CCCACCA | | CUGGACU | | |
| tRNA^{Ala(UGC)} | HC230 | 181 | GGGGAUAUAGCU | 413 | CUACCAACUGAGC | 22 | 5'-t |
| | | | CAGUUGGUAG | | UAUAUCCCC | | |
| | HC231 | 182 | GGGGAUAUAGCU | 414 | CCAACUGAGCUAU | 19 | |
| | | | CAGUUGG | | AUCCCC | | |
| | HC232 | 183 | UCGAGUCCGCUU | 415 | UGGUGGAGAUAA | 22 | 3'-t |
| | | | AUCUCCACCA | | GCGGACUCGA | | |
| | HC233 | 184 | AGUCCGCUUAUC | 416 | UGGUGGAGAUAA | 19 | |
| | | | UCCACCA | | GCGGACU | | |
| tRNA^{Lys(UUU)}_2 | HC234 | 185 | GGGUGUAUAGCU | 417 | CUACCAACUGAGC | 22 | 5'-t |
| | | | CAGUUGGUAG | | UAUACACCC | | |
| | HC235 | 186 | GGGUGUAUAGCU | 418 | CCAACUGAGCUAU | 19 | |
| | | | CAGUUGG | | ACACCC | | |
| | HC236 | 187 | UCAAGUCCUGCU | 419 | UGGUGGGUAUAG | 22 | 3'-t |
| | | | AUACCCACCA | | CAGGACUUGA | | |
| | HC237 | 188 | AGUCCUGCUAUA | 420 | UGGUGGGUAUAG | 19 | |
| | | | CCCACCA | | CAGGACU | | |
| tRNA^{Lys(CUU)} | HC238 | 189 | CACCCUGUAGCU | 421 | UCUUCCUCUGAG | 22 | 5'-t |
| | | | CAGAGGAAGA | | CUACAGGGUG | | |
| | HC239 | 190 | CACCCUGUAGCU | 422 | UCCUCUGAGCUA | 19 | |
| | | | CAGAGGA | | CAGGGUG | | |
| | HC240 | 191 | UCAAGUCCUACC | 423 | UGGGUAACCUGG | 22 | 3'-t |
| | | | AGGUUACCCA | | UAGGACUUGA | | |
| | HC241 | 192 | AGUCCUACCAGG | 424 | UGGGUAACCUGG | 19 | |
| | | | UUACCCA | | UAGGACU | | |
| tRNA^{Gln(UUG)}_2 | HC242 | 193 | UGGAGUAUAGCC | 425 | CUUACCACUUGG | 22 | 5'-t |
| | | | AAGUGGUAAG | | CUAUACUCCA | | |
| | HC243 | 194 | UGGAGUAUAGCC | 426 | ACCACUUGGCUAU | 19 | |
| | | | AAGUGGU | | ACUCCA | | |
| | HC244 | 195 | UCGAAUCCUUUU | 427 | UGGCUGGAGUAA | 22 | 3'-t |
| | | | ACUCCAGCCA | | AAGGAUUCGA | | |
| | HC245 | 196 | AAUCCUUUUACU | 428 | UGGCUGGAGUAA | 19 | |
| | | | CCAGCCA | | AAGGAUU | | |
| tRNA^{Met(CAU)}_3 | HC246 | 197 | GGGCUUAUAGUU | 429 | CAACCAAUUAAAC | 22 | 5'-t |
| | | | UAAUUGGUUG | | UAUAAGCCC | | |
| | HC247 | 198 | GGGCUUAUAGUU | 430 | CCAAUUAAACUAU | 19 | |
| | | | UAAUUGG | | AAGCCC | | |
| | HC248 | 199 | UCGAGCCCUACU | 431 | UGGUAGGCUUAG | 22 | 3'-t |
| | | | AAGCCUACCA | | UAGGGCUCGA | | |
| | HC249 | 200 | AGCCCUACUAAG | 432 | UGGUAGGCUUAG | 19 | |
| | | | CCUACCA | | UAGGGCU | | |
| tRNA^{Met(CAU)}_4 | HC250 | 201 | GCAUCCAUGGCU | 433 | UUAACCAUUCAGC | 22 | 5'-t |
| | | | GAAUGGUUAA | | CAUGGAUGC | | |
| | HC251 | 202 | GCAUCCAUGGCU | 434 | ACCAUUCAGCCAU | 19 | |
| | | | GAAUGGU | | GGAUGC | | |
| | HC252 | 203 | UCAAUUCCUACU | 435 | UGGUGCAUCCAG | 22 | 3'-t |
| | | | GGAUGCACCA | | UAGGAAUUGA | | |
| | HC253 | 204 | AUUCCUACUGGA | 436 | UGGUGCAUCCAG | 19 | |
| | | | UGCACCA | | UAGGAAU | | |
| tRNA^{Tyr(GUA)}_2 | HC254 | 205 | GGGAGAGUGGCC | 437 | UUGACCACUCGG | 22 | 5'-t |
| | | | GAGUGGUCAA | | CCACUCUCCC | | |
| | HC255 | 206 | GGGAGAGUGGCC | 438 | ACCACUCGGCCAC | 19 | |
| | | | GAGUGGU | | UCUCCC | | |
| | HC256 | 207 | UCGAAUCCUGCC | 439 | UGGUGGGAGAGG | 22 | 3'-t |
| | | | UCUCCCACCA | | CAGGAUUCGA | | |
| | HC257 | 208 | AAUCCUGCCUCU | 440 | UGGUGGGAGAGG | 19 | |
| | | | CCCACCA | | CAGGAUU | | |
| tRNA^{Ser(GCU)}_2 | HC258 | 209 | GGAGGUAUGGCU | 441 | UAAGCCACUCAGC | 22 | 5'-t |
| | | | GAGUGGCUUA | | CAUACCUCC | | |
| | HC259 | 210 | GGAGGUAUGGCU | 442 | GCCACUCAGCCAU | 19 | |
| | | | GAGUGGC | | ACCUCC | | |
| | HC260 | 211 | UCGAAUCCCAUU | 443 | UGGCGGAGGAAA | 22 | 3'-t |
| | | | UCCUCCGCCA | | UGGGAUUCGA | | |
| | HC261 | 212 | AAUCCCAUUUCC | 444 | UGGCGGAGGAAA | 19 | |
| | | | UCCGCCA | | UGGGAUU | | |
| tRNA^{Phe(GAA)}_3 | HC262 | 213 | GUUCAGGUAGCU | 445 | UAACCAGCUGAGC | 22 | 5'-t |
| | | | CAGCUGGUUA | | UACCUGAAC | | |
| | HC263 | 214 | GUUCAGGUAGCU | 446 | CCAGCUGAGCUA | 19 | |
| | | | CAGCUGG | | CCUGAAC | | |
| | HC264 | 215 | UCGAAUCCACUU | 447 | UGGCGCUUAGAA | 22 | 3'-t |
| | | | CUAAGCGCCA | | GUGGAUUCGA | | |
| | HC265 | 216 | AAUCCACUUCUA | 448 | UGGCGCUUAGAA | 19 | |
| | | | AGCGCCA | | GUGGAUU | | |
| tRNA^{Phe(AAA)} | HC266 | 217 | GUAACGAUCGAA | 449 | ACUUCCAUUAUUC | 22 | 5'-t |
| | | | UAAUGGAAGU | | GAUCGUUAC | | |
| | HC267 | 218 | GUAACGAUCGAA | 450 | UCCAUUAUUCGAU | 19 | |
| | | | UAAUGGA | | CGUUAC | | |
| | HC268 | 219 | UCAAAUCCAAUU | 451 | UGGAGUAACGAAU | 22 | 3'-t |
| | | | CGUUACUCCA | | UGGAUUUGA | | |
| | HC269 | 220 | AAUCCAAUUCGU | 452 | UGGAGUAACGAAU | 19 | |
| | | | UACUCCA | | UGGAUU | | |
| tRNA^{Pro(UGG)}_3 | HC270 | 221 | AGGGAUGUAGCG | 453 | UACCAAGCUGCG | 22 | 5'-t |
| | | | CAGCUUGGUA | | CUACAUCCCU | | |
| | HC271 | 222 | AGGGAUGUAGCG | 454 | CAAGCUGCGCUA | 19 | |
| | | | CAGCUUG | | CAUCCCU | | |
| | HC272 | 223 | UCCAAUCCUGUC | 455 | UGGUAGGGAUGA | 22 | 3'-t |
| | | | AUCCCUACCA | | CAGGAUUGGA | | |
| | HC273 | 224 | AAUCCUGUCAUC | 456 | UGGUAGGGAUGA | 19 | |
| | | | CCUACCA | | CAGGAUU | | |
| tRNA^{Ile(CAU)} | HC274 | 225 | GGGCUAUUAGCU | 457 | UCUACCACUGAGC | 22 | 5'-t |
| | | | CAGUGGUAGA | | UAAUAGCCC | | |
| | HC275 | 226 | GGGCUAUUAGCU | 458 | ACCACUGAGCUAA | 19 | |
| | | | CAGUGGU | | UAGCCC | | |
| | HC276 | 227 | UCAAGUCCAGGA | 459 | UGGUGGGCCAUC | 22 | 3'-t |
| | | | UGGCCCACCA | | CUGGACUUGA | | |
| | HC277 | 228 | AGUCCAGGAUGG | 460 | UGGUGGGCCAUC | 19 | |
| | | | CCCACCA | | CUGGACU | | |
| tRNA^{Gly(GCC)}_3 | HC278 | 229 | GCGGAAAUAGCU | 461 | UCUACCAUUAAGC | 22 | 5'-t |
| | | | UAAUGGUAGA | | UAUUUCCGC | | |
| | HC279 | 230 | GCGGAAAUAGCU | 462 | ACCAUUAAGCUAU | 19 | |
| | | | UAAUGGU | | UUCCGC | | |
| | HC280 | 231 | UCAAGUCCCUCC | 463 | UGGAGCGGAAGG | 22 | 3'-t |
| | | | UUCCGCUCCA | | AGGGACUUGA | | |
| | HC281 | 232 | AGUCCCUCCUUC | 464 | UGGAGCGGAAGG | 19 | |
| | | | CGCUCCA | | AGGGACU | | |

The inventors unexpectedly found that the RNA molecules isolated or derived from a plant of genus Panax in particular Panax ginseng C. A. Mey are effective on protecting cardiomyocytes, in particular they are capable of promoting the growth, proliferation and/or metastasis of cardiomyocytes.

Turning back to the method of treatment, the method comprises the step of administering an effective amount of RNA molecule as described above to the subject suffering from heart diseases. In an embodiment, the step of administering the RNA molecule to the subject comprises contacting cardiomyocytes of the subject with the RNA molecule.

The term “CHD” describes a physiological condition in subjects in which heart arteries are narrowed, less blood and oxygen reach the heart muscle. In an embodiment, the CHD to be treated is atherosclerosis, angina, heart attack and myocardial infarction. In a particular embodiment, the CHD is myocardial infarction. Accordingly, the method of the present invention can be applied to treat a subject suffering from a coronary heart disease and related disorders.

The term “subject” used herein refers to a living organism and can include but is not limited to a human and an animal. The subject is preferably a mammal, preferably a human. The RNA molecules may be administered through injection to the subject, preferably a human. The term injection encompasses intravenous, intramuscular, subcutaneous and intradermal administration. In an embodiment, the RNA molecule of the present invention is administered together with suitable excipient(s) to the subject through intravenous injection. For instance, the RNA molecule may be delivered to the subject or cells via transfection, electroporation or viral-mediated delivery.

The expression “effective amount” generally denotes an amount sufficient to produce therapeutically desirable results, wherein the exact nature of the result varies depending on the specific condition which is treated. In this invention, CHD is the condition to be treated and therefore the result is usually a promotion or protection of the growth and proliferation of cardiomyocytes, a protection or amelioration of symptoms related to CHD. In an embodiment, where the injury is hypoxia/reoxygenation (ischemia/reperfusion) injury, the result is usually a promotion of the growth and proliferation of cardiomyocytes, relief of destruction of the cytoskeleton or amelioration of symptoms related to injured cardiomyocytes.

The RNA molecule of the present invention may be administered in form of a pharmaceutical composition comprising the RNA molecule and at least one pharmaceutically tolerable excipient. The pharmaceutically tolerable excipient may be one or more of a diluent, a filler, a binder, a disintegrant, a lubricant, a coloring agent, a surfactant, a gene delivery carrier and a preservative. The pharmaceutical composition can be present in solid, semisolid or liquid form, preferably in liquid form. The pharmaceutical composition can be liposome freeze-dried powder, polypeptide nanometer freeze-dried powder, spray and tablets. The pharmaceutical composition may comprise further pharmaceutical effective ingredients such as therapeutic compounds which are used for treating CHD such as Rg1. The skilled technician is able to select suitable pharmaceutically tolerable excipients depending on the form of the pharmaceutical composition and is aware of methods for manufacturing pharmaceutical compositions as well as able to select a suitable method for preparing the pharmaceutical composition depending on the kind of pharmaceutically tolerable excipients and the form of the pharmaceutical composition.

In an embodiment, RNA molecules provided as a composition containing a gene delivery vector. A gene delivery vector is any molecule that act as a carrier to deliver a gene to a cell. In embodiments where RNA molecules are transfected into cells, gene delivery vectors are considered to be transfection agents. In the embodiment of delivering RNA molecules by a recombinant viral vector, the gene delivery vector is a viral vector carrying a double-stranded RNA molecule describe above in the present invention. Gene delivery vectors include but are not limited to vectors such as virus vectors, collagens such as terminated peptide collagens, polymers such as polyetenimine (PEI), polypeptides such as poly (L-lysine) and protamine, and liposomes such as Lipofectamine. Gene delivery vectors can be commercially available, such as transfection reagents from Thermo Fisher, U.S.A. including Lipofectamine RNAiMAX, Lipofectamine 3000, Lipofectamine 2000 and DharmaFECT series from Dharmacon; RNAi-Mate from GenePharma, China; terminated peptide collagens from Koken Co. Ltd, Japan; and Histidine-lysine peptide copolymer from siRNAomics, China. Gene delivery vectors can be viral vectors based on retroviruses, adeno-associated viruses, adenoviruses, and lentiviruses. The gene delivery vector should be of low toxicity and not induce significant immune response in subjects. In an embodiment, the pharmaceutical composition may further comprise a nucleic acid stabilizer. The nucleic acid stabilizer refers to any chemicals that are capable of maintaining the stability of the RNA molecule in the composition to minimize or avoid degradation, in particular those having ability to deactivate activity of nucleases or the like degrading the RNA molecules.

Accordingly, the present invention also pertains to a pharmaceutical composition as described above, in particular comprising the RNA molecule and a pharmaceutically tolerable excipient as defined above. In an embodiment, the RNA molecule comprises at least one sequence selected from SEQ ID NO: 1 to 232 or a functional variant or homologue thereof.

Preferably, the RNA molecule is isolated or derived from a plant of the genus Panax as described above, in particular from Panax ginseng C. A. Mey.

The RNA molecules of the present invention are also suitable for promoting the growth and proliferation of cardiomyocytes. In another aspect of the invention, there is provided a method of promoting the growth and proliferation of cardiomyocytes comprising a step of contacting said cells with an effective amount of a RNA molecule as defined above. Preferably the RNA molecule is isolated or derived from a plant of the genus Panax or comprises a sequence selected from SEQ ID NO: 1 to SEQ ID NO: 232 or a functional variant or homologue thereof.

In an embodiment, the RNA molecule has a sequence length of from about 50 to 200 nucleotides, more preferably has a length of from about 60 to about 150 nucleotides, in particular from about 70 to about 100 nucleotides. The RNA molecule is a non-coding molecule preferably a transfer RNA molecule. Preferably, the RNA molecule comprises a sequence selected from SEQ ID NO: 465 to SEQ ID NO: 522 or a functional variant or homologue thereof; or the RNA molecule comprises SEQ ID NO: 465 to SEQ ID NO: 468 or a functional variant or homologue thereof; or the RNA molecule consists of a sequence selected from SEQ ID NO: 465 to SEQ ID NO: 522 or SEQ ID NO: 465 to SEQ ID NO: 468 or a functional variant or homologue thereof.

In an alternative embodiment, the RNA molecule has a sequence length of from about 10 to about 30 base pairs, from about 15 to about 25 base pairs, from about 19 to about 22 base pairs, 19 base pairs or 22 base pairs. Preferably, the RNA molecule is a double-stranded RNA molecule comprising a sense sequence selected from SEQ ID NO: 1 to SEQ ID NO: 232 or a functional variant or homologue thereof, in particular SEQ ID NO: 1 to SEQ ID NO: 40 or a functional variant or homologue thereof; or consists of a sequence selected from SEQ ID NO: 1 to SEQ ID NO: 232, in particular SEQ ID NO: 1 to SEQ ID NO: 40 or a functional variant or homologue thereof. The double-stranded RNA molecule comprises a complementary antisense sequence. The RNA molecule may further comprise 2 mer of 3′ overhangs.

The step of contacting the cardiomyocytes with the RNA molecule of the present invention may be carried out by applying a composition in particular an incubation solution comprising the RNA molecule to said cardiomyocytes which incubation solution may further comprise suitable excipients as defined above, a buffer or a suitable growth medium. In such embodiment of the present invention, the cardiomyocytes are taken from a subject such as an animal or human, in particular a human. The RNA molecule is provided in the composition at a concentration of at least 0.3 nM, at least 3 nM, from about 0.3 nM to about 900 nM, from about 10 nM to about 100 nM, or from about 50 nM to about 300 nM. In addition, excipients may include gene delivery vectors, such as, but not limited to, collagen-based vectors or liposome formers.

In addition to the above, the present invention pertains to a double-stranded RNA molecule as described above, i.e. comprising a sense sequence selected from SEQ ID NO: 1 to SEQ ID NO: 232 or a functional variant or homologue thereof, and a complementary antisense sequence. In particular, the double-stranded RNA molecule consists of a sense sequence selected from SEQ ID NO: 1 to SEQ ID NO: 232 or a functional variant or homologue thereof, a complementary antisense sequence selected from SEQ ID NO: 233 to SEQ ID NO: 464, and optionally a 3′ overhang. Example embodiments of the double-stranded RNA molecule are presented in Table 2. The double-stranded RNA may be subject to modification and therefore may carry at least one modified nucleoside selected form inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

In further aspect of the invention, there is provided a vector comprising a nucleic acid molecule, wherein the nucleic acid molecule is a RNA molecule as described above. In particular, the RNA molecule having a sequence selected from SEQ ID NO: 1 to SEQ ID NO: 232 or a functional variant or homologue thereof. In an embodiment, the vector is a recombinant vector comprising the double-stranded RNA molecule as described above. The vector may be viral-based vector derived from retrovirus, adeno-associated virus, adenovirus, or lentivirus. An ordinary skilled in the art would appreciate suitable approach to incorporate the RNA molecule of the present invention into a vector.

Still further, the present invention pertains to use of a nucleic acid molecule in the preparation of a medicament for treating CHD. The nucleic acid is a RNA molecule as described above including a functional variant or homologue thereof. It would also be appreciated that the RNA molecule of the present invention can be used as a small interfering RNA molecule to interfere the expression of certain genes in the target CHD, thereby to cause gene silencing, inhibition of apoptosis and injury, or the like to achieve the desired therapeutic effect.

Accordingly, the present invention provides a novel and effective approach for treating CHD from various origins by administration of a RNA molecule that is isolated or derived from a plant of the genus Panax, or in particular a RNA molecule comprising a sequence selected from SEQ ID NO: 1 to 232. Administration of said RNA molecule is also suitable for promoting the growth and proliferation of cardiomyocytes. The RNA molecules are found to be highly effective at promoting the growth and proliferation of cardiomyocytes in vitro.

The invention is now described in the following non-limiting examples.

### EXAMPLES

### Chemicals and Materials

Fresh roots of Panax ginseng C. A. Mey were collected from Fusong Town in the year of 2017 from Jilin Province, China. Cetrimonium bromide (CTAB) and sodium chloride were purchased from Kingdin Industrial Co., Ltd. (Hong Kong, China). Water-saturated phenol was purchased from Leagene Co., Ltd. (Beijing, China). Chloroform and ethanol were purchased from Anaqua Chemicals Supply Inc. Ltd. (U.S.A.). Isopentanol and guanidinium thiocyanate were purchased from Tokyo Chemical Industry CO., Ltd. (Japan). Tris-HCl and ethylenediaminetetraacetic acid (EDTA) were purchased from Acros Organics (U.S.A), low range ssRNA ladder was purchased from New England Biolabs (Beverly, Mass., U.S.A.). TRIzol® Reagent (Invitrogen), mirVana™ miRNA isolation kit, SYBR gold nucleic acid gel stain and gel loading buffer II were purchased from Thermo Fisher Scientific (U.S.A.). 40% acrylamide/bis solution (19:1), tris/borate/EDTA (TBE), ammonium persulphate (APS) and tetramethylethylenediamine (TEMED) were purchased from Biorad Laboratories Inc. (U.S.A). Rat cardiomyocyte cell line (H9C2) were purchased from ATCC (Manassas, Va., U.S.A.). Opti-MEM I Reduced Serum Media, Dulbecco's Modified Eagle Medium (DMEM), Glucose free Dulbecco's Modified Eagle Medium (glucose free DMEM), Fetal Bovine Serum (FBS), Penicillin-Streptomycin were purchased from Gibco (Life Technologies, Auckland, New Zealand). 3-(4,5-Dimethylthiazol-2-yl)-2,5-Diphenyltetrazolium Bromide (MTT) and DAPI was purchased from Sigma (St. Louis, Mo., U.S.A.). Mitochondrial viability stain solution was purchased from Abcam (Cambridge, England). Rhodamine Phalloidin was purchased from Cytoskeleton, Inc. (Denver, U.S.A.).

### Example 1

Isolation of RNA molecules from a plant of genus Panax Roots of Panax ginseng C. A. Mey were freshly collected and immediately stored in liquid nitrogen until use. RNAs having a length of 200 nucleotides or below, i.e. small RNAs species, were extracted from Panax ginseng C. A. Mey by using a polysaccharase-aided RNA isolation (PARI) method, which method is described for the first time. Briefly, plant tissues were ground into a fine powder in liquid nitrogen and then homogenized in TRIzol reagent using a digital dispersing device (IKA, Germany). After fully lysed for 10 min at room temperature, an equal volume of chloroform was added and followed by centrifugation at 12,000×g for 15 min at 4° C. The supernatant was collected and precipitated by adding 1/25 volume of 5 M sodium chloride and 1.25 volume of cold absolute ethanol, and stored at −20° C. for 30 min. Then precipitation was hydrolyzed by polysaccharase, until the pellet was completely dissolved. The hydrolysate was mixed with 2×CTAB buffer, and extracted with an equal volume of phenol:chloroform:isopentanol (50:48:1) by vortexing vigorously. Phases were separated at 4° C. by centrifugation at 12,000×g for 15 min and the supernatant was extracted again as described above with chloroform:isopentanol (24:1). The supernatant was collected and mixed with an equal volume of 6 M guanidinium thiocyanate, followed by adding 100% ethanol to a final concentration of 55%. The mixture was passed through a filter cartridge containing a silica membrane, which immobilizes the RNAs. The filter was then washed for several times with 80% (v/v) ethanol solution, and finally all RNAs were eluted with a low ionic-strength solution or RNase-free water. The small RNA species were isolated and enriched by using a mirVana™ miRNA isolation kit following the manufacturer's instruction.

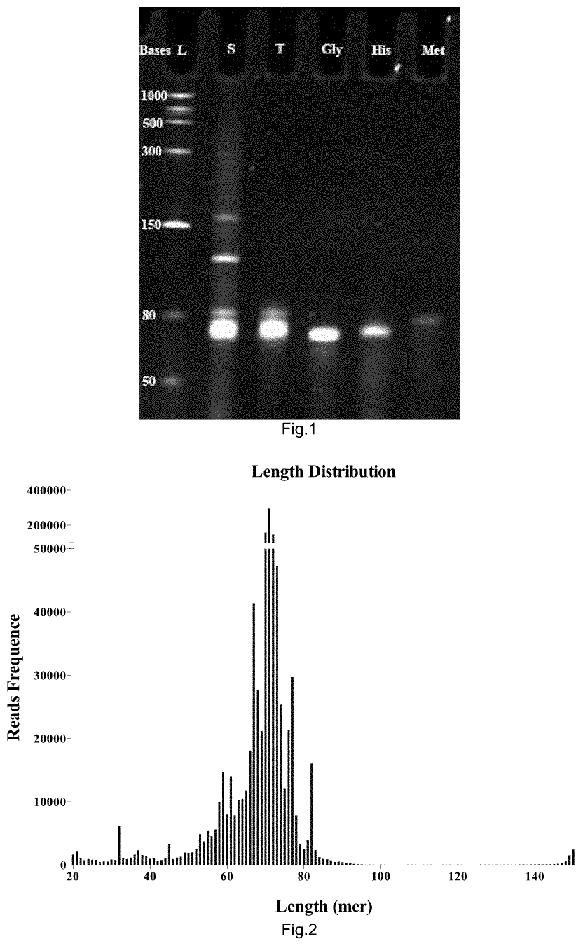

Further, the total tRNAs in the isolated small RNA species were separated by electrophoresis in 6% polyacrylamide TBE gels containing 8 M urea prepared according to the manufacturer's protocol (Biorad, U.S.A.). After staining with SYBR Gold nucleic acid gel stain, polyacrylamide gels were examined using a UV lamp and the region of gels containing total tRNAs were cut off by using a clean and sharp scalpel. FIG. 1 shows gel electrophoresis profiles of small RNA species from Panax ginseng C. A. Mey, including low range ssRNA Ladder, small RNA molecules, transfer RNAs and individual transfer RNA including tRNA^{Gly(GCC)}, tRNA^{His(GUG)}, tRNA^{Met(CAU)}. The band was sliced into small pieces and the total tRNAs were recovered from the gel by electroelution in a 3 kD molecular weight cut-off dialysis tubing (Spectrum, C.A.) at 100 V for 50 min in 1×TAE buffer. The eluents in the dialysis tubing were recovered and the total tRNAs were desalted and concentrated by using the mirVana™ miRNA isolation kit. The quality and purity of the RNA products were then confirmed using a Nanodrop Spectrophotometer (Thermo Scientific, U.S.A.) and Agilent 2100 Bioanalyzer (Agilent, U.S.A.).

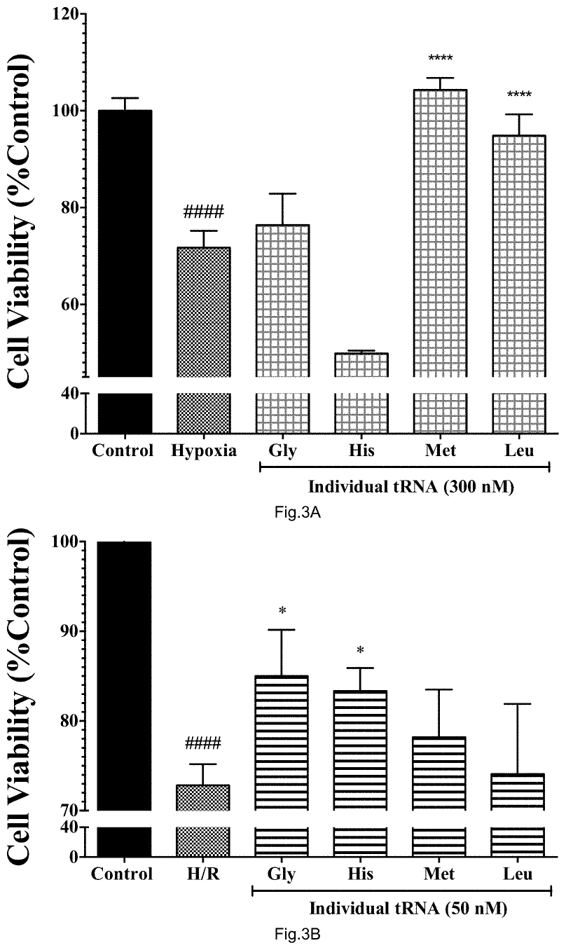

The inventors then constructed the total tRNAs library and performed sequencing. Sequencing libraries were generated by using TruSeq small RNA Library Preparation Kit (Illumina, U.S.A.), followed by a round of adaptor ligation, reverse transcription and PCR enrichment. PCR products were then purified and libraries were quantified on the Agilent Bioanalyzer 2100 system (Agilent Technologies, U.S.A.). The library preparations were sequenced at the Novogene Bioinformatics Institute (Beijing, China) on an Illumina HiSeq platform using the 150 bp paired-end (PE150) strategy to generate over 15 million raw paired reads. U.S. Pat. No. 5,772,569 clean reads were obtained by removing low quality regions and adaptor sequences. FIG. 2 is a bar chart showing read length distribution of tRNAs. The tRNA genes were identified by using the tRNAscan-SE 2.0 program (http://lowelab.ucsc.edu/tRNAscan-SE/) and annotated by searching the Nucleotide Collection (nr/nt) database using Basic Local Alignment Search Tool (BLAST) program (https://blast.ncbi.nlm.nih.gov/Blast.cgi). 58 tRNA sequences from Panax ginseng C. A. Mey were identified and listed in Table 1.

Each of the tRNAs was then isolated from a mixture of small RNAs (<200 mer) from Panax ginseng C. A. Mey by immobilization of the target tRNAs onto the streptavidin-coated magnetic beads with specific biotinylated capture DNA probes. To bind specific tRNA molecules, a corresponded single stranded DNA oligonucleotide (20 to 45-mer) were synthesized, which was designed based on the sequence information of Illumina sequencing and should be complementary to a unique segment of the target tRNA. Cognate DNA probes were incubated with small RNA mixture for about 1.5 h in annealing buffer and allowed to hybridize to the targeted tRNA molecules in solution at the proper annealing temperatures that were generally 5° C. lower than the melting temperature (Tₘ). Streptavidin-coated magnetic beads were then added to the mixture and incubated for 30 min at the annealing temperatures. After the hybridized sequences are immobilized onto the magnetic beads via the streptavidin-biotin bond, the biotinylated DNA/tRNA coated beads were separated with a magnet for 1-2 min and washed 3-4 times in washing buffer at 40° C. The magnetic beads were resuspended to a desired concentration in RNase-free water and thereby to release the immobilized tRNA molecules by incubation at 70° C. for 5 min. Accordingly, the isolated and purified tRNA molecules of SEQ ID NO: 465 to 522 were obtained.

### Example 2

### Synthesis of RNA Molecules

The inventors designed and synthesized RNA molecules having a length of about 19 to 22 bp based on the 58 isolated tRNA sequences in Example 1. In particular, the tRNA sequences are considered to have at least 3 portions, namely a 5′-terminal portion (5′-t), a 3′-terminal portion (3′-t) and an anticodon portion. Each of the specifically designed RNA molecules contains any one of the portions. For instance, designed RNA molecules containing a 5′ terminal portion of the corresponding full-length tRNA sequence are referred as 5′-t group RNA molecules; designed RNA molecules containing a 3′ terminal portion of the corresponding full-length tRNA sequence are referred as 3′-t group RNA molecules; designed RNA molecules containing an anticodon portion of the corresponding full-length tRNA sequence are referred as anticodon group RNA molecules. The RNA molecules having a sense sequence selected from SEQ ID NO: 1 to SEQ ID NO: 232 and a complementary antisense sequence selected from SEQ ID NO: 233 to SEQ ID NO: 464, as shown in Table 2, were designed and synthesized by cleavage at different sites on the tRNA sequences in Table 1.

### Example 3

### Cardioprotective Effect of RNA Molecules on Cardiomyocytes

H9C2 cell lines were cultured in Dulbecco's Modified Eagle Medium (DMEM) containing 10% FBS and 1% penicillin/streptomycin at humidified atmosphere containing 5% CO₂ at 37° C.

In the cell viability assay or mitochondrial viability assay, exponentially growing cells of H9C2 cell line were plated in 96-well microplate at a density of 5000 cells per well in 100 μL of culture medium and allowed to adhere for 24 h before treatment. For hypoxia, hypoxic treatment was achieved by exposing cells to KRB buffer (composition in: NaCl 115 mM, KCl 4.7 mM, CaCl₂ 2.5 mM, KH₂PO₄ 1.2 mM, MgSO₄ 1.2 mM, NaHCO₃ 24 mM, HEPES 10 mM; pH 7.4) at 37° C. for 3 hr in an oxygen-free hypoxic chamber (Stem Cell Technologies, United States), serial concentrations of RNA molecules obtained in Example 1 were then added to the cells before hypoxic treatment. For hypoxia/reoxygenation (H/R), Hypoxic treatment was achieved by exposing cells in glucose-free DMEM under conditions of 94.9% N₂/5% CO₂/0.1% O₂ for 12 hr at a hypoxystation (whitley H35 hypoxystation, Don Whitley Scientific Ltd., England), serial concentrations of RNA molecules obtained in Example 1 and 2 were then added to the cells and reoxygenation by incubating in the normoxic condition (95% air/5% CO₂) at 37° C. for 6 hr. After hypoxia or hypoxia/reoxygenation, MTT solution (100 μL per well, 0.5 mg/mL solution) or mitochondrial viability stain solution (follow the manufacture's instruction) was added to each well and incubated for 4 h at 37° C. Subsequently, for cell viability assay, 150 μL dimethyl sulfoxide (DMSO) were added and the optical densities of the resulting solutions were calorimetrically determined at 570 nm using a SpectraMax Paradigm multi-mode microplate reader (Molecular Devices, Sunnyvale, Calif., U.S.A). For mitochondrial viability assay, fluorescence detected at 550 nM excitation and 590 nM emission using SpectraMax Paradigm multi-mode microplate reader. Dose-response curves were obtained and calculated by GraphPad Prism 6 (GraphPad, La Jolla, Calif., USA). Each experiment was carried out for three times and expressed as means±standard deviation.

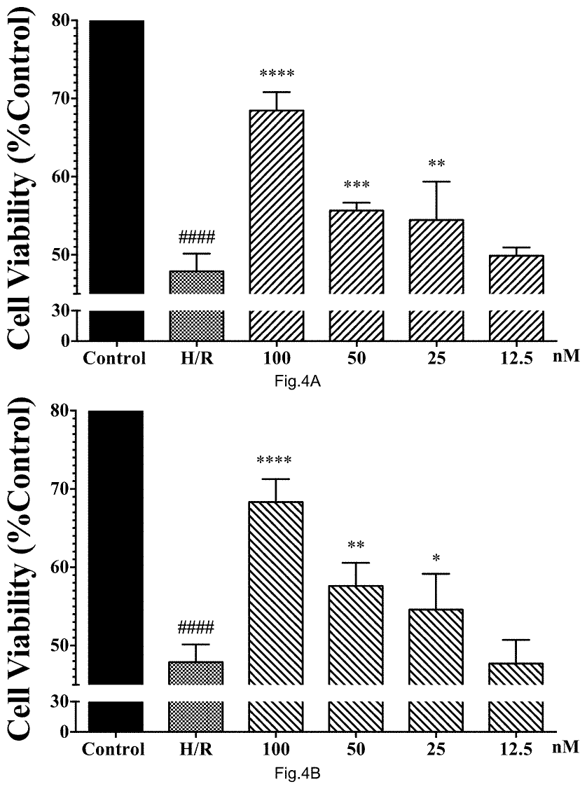

With reference to FIG. 3A, H9C2 cells were treated with 300 nM RNA molecules of tRNA^{Gly(GCC)}, tRNA^{His(GUG)}, tRNA^{Met(CAU)} and tRNA^{Leu(CAA)}, i.e. SEQ ID NO: 465 to 468, and cultured under hypoxia before addition of MTT solution. The cell viability of these cells is compared to a control group and a hypoxia group. The results show that tRNA^{Gly(GCC)}, tRNA^{Met(CAU)} and tRNA^{Le(CAA)} are capable to promote the growth and proliferation of cardiomyocytes, indicating these RNA molecules can protect cardiomyocytes from hypoxic injury.

With reference to FIG. 3B, H9C2 cells were treated with 50 nM RNA molecules of tRNA^{Gly(GCC)}, tRNA^{His(GUG)}, tRNA^{Met(CAU)} and tRNA^{Leu(CAA)}, i.e. SEQ ID NO: 465 to 468, and cultured under hypoxia/reoxygenation before addition of MTT solution. The cell viability of these cells is compared to a control group and a H/R group. The results show that tRNA^{Gly(GCC)}, tRNA^{His(GUG)} molecules are capable to promote the growth and proliferation of cardiomyocytes, indicating these RNA molecules can protect cardiomyocytes from hypoxia/reoxygenation (H/R) injury.

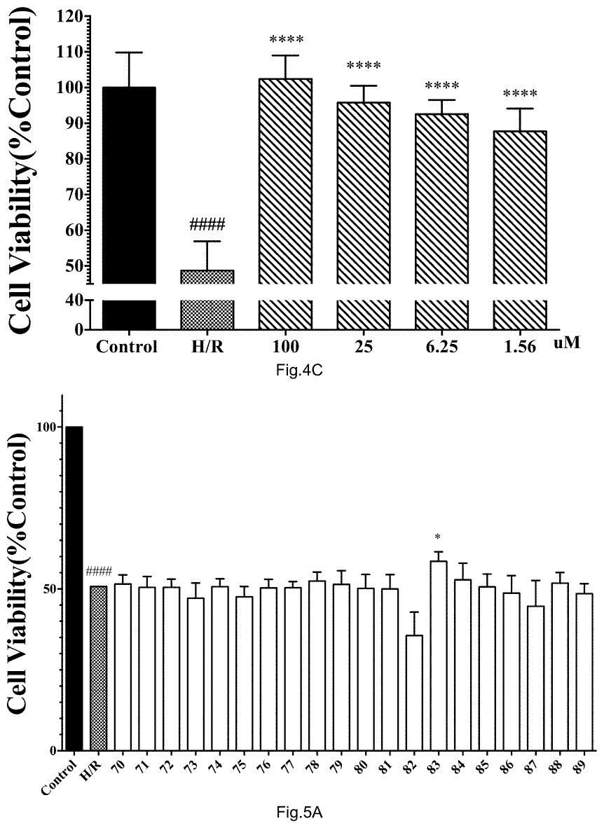

FIG. 4A shows the cardioprotective effect of tRNA^{His(GUG)}, i.e. SEQ ID NO: 465, on H9C2 cells. Different concentrations of tRNA^{His(GUG)} were used, i.e. 100 nM, 50 nM, 25 nM, and 12.5 nM, and compared to a control group and a H/R group. It is shown that the tRNA^{His(GUG)} on cardiomyocytes in particular H9C2 cells exhibit significant cardioprotective effects in a dose-dependent manner.

FIG. 4B shows the cardioprotective effect of tRNA^{Gly(GCC)}, i.e. SEQ ID NO: 466, on H9C2 cells. Different concentrations of tRNA^{Gly(GCC)} were used, i.e. 100 nM, 50 nM, 25 nM, and 12.5 nM, and compared to a control group and a H/R group. It is shown that the tRNA^{Gly(GCC)} on cardiomyocytes in particular H9C2 cells exhibit significant cardioprotective effects in a dose-dependent manner.

A comparative example of ginsenoside Rg1 implementation was used, and the results were shown in FIG. 4C.

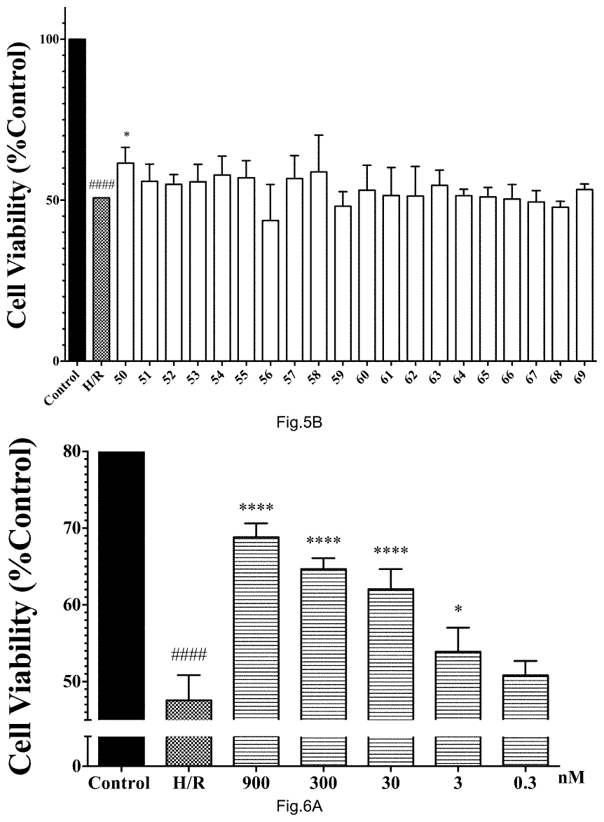

FIG. 5A and FIG. 5B show the cardioprotective effect of RNA molecules synthesized in Example 2 on H9C2 cells, in particular those having sense sequence of SEQ ID NO: 1 to 40. The results show that the RNA molecules HC50 and HC83 are effective in promoting the growth and proliferation of cardiomyocytes in particular H9C2 cells in this example. In other words, RNA molecules HC50 and HC83 are useful in protecting cardiomyocytes from hypoxia/reoxygenation (H/R) injury.

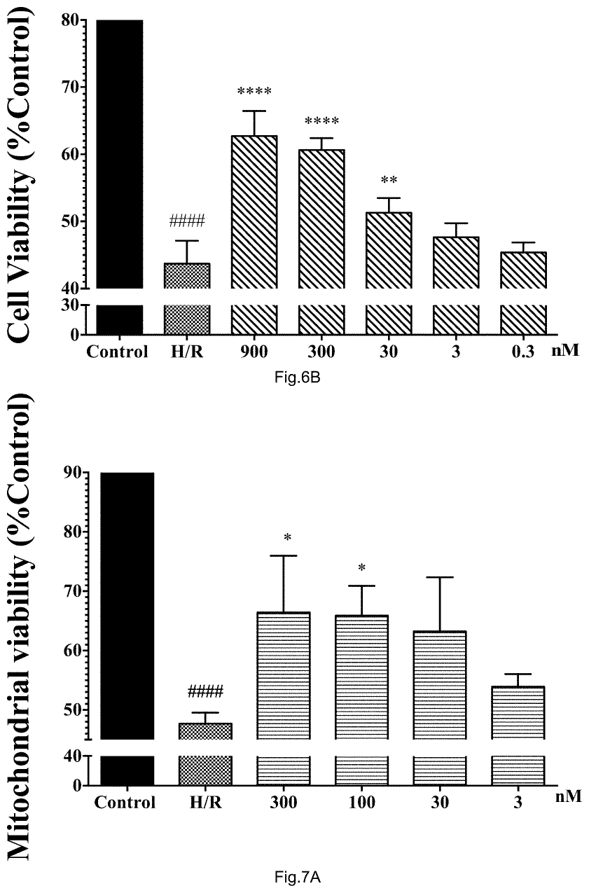

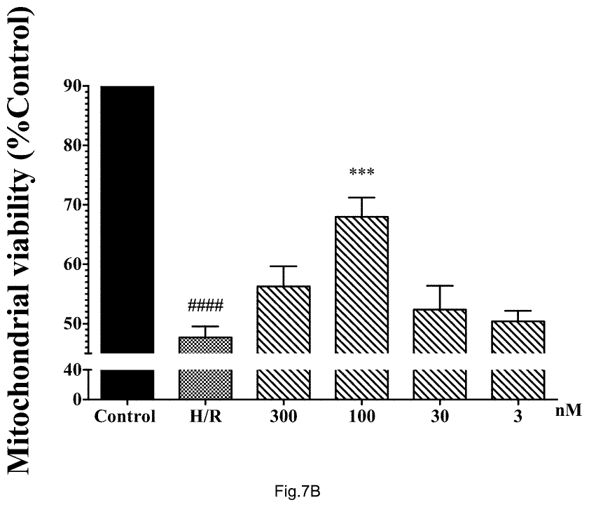

The inventors then specifically determined the cardioprotective effect of RNA molecule HC50 and HC83 on H9C2 cells, at different concentrations, i.e. 900 nM, 300 nM, 30 nM, 3 nM and 0.3 nM. As shown in FIG. 6A, FIG. 6B, FIG. 7A and FIG. 7B, the results are compared to a control group and a H/R group. The results demonstrated that RNA molecule HC50 and HC83 has a dose-dependent protective effect against hypoxia/reoxygenation (H/R) injury.

### Example 4

### Cytoskeleton Protection of RNA Molecules on Cardiomyocytes

H9C2 cells were plated in p-slide 8 well plate (Ibidi GmbH, Germany) at a density of 10000 cells per well in 200 μL of culture medium and allowed to adhere for 24 h before treatment. Hypoxic treatment was achieved by exposing cells in glucose-free DMEM under conditions of 94.9% N₂/5% CO₂/0.1% O₂ for 12 hr at a hypoxystation (whitley H35 hypoxystation, Don Whitley Scientific Ltd., England), serial concentrations of RNA molecules obtained in Example 2 were then added to the cells and reoxygenation by incubating in the normoxic condition (95% air/5% CO₂) at 37° C. for 6 hr. After hypoxia/reoxygenation, cells were stained with Rhodamine Phalloidin and DAPI following the manufacturer's instruction. Images were acquired on a Leica TCS SP8 Confocal Microscopy with a 20× objective.

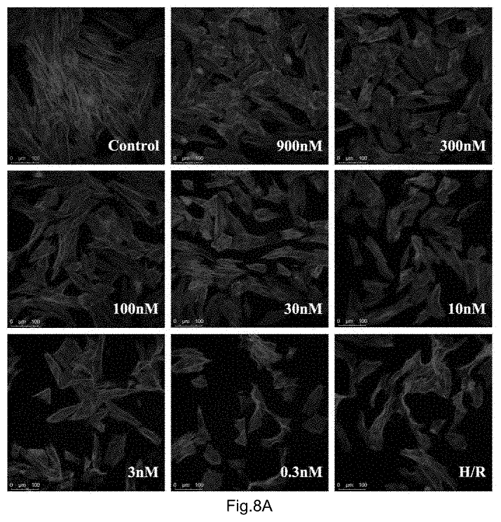

The inventors specifically determined the protective effects of RNA molecule HC50 and HC83 on H9C2 cell cytoskeleton at different concentrations, i.e. 900 nM, 300 nM, 100 nM, 30 nM, 10 nM, 3 nM, and 0.3 nM. With reference to FIG. 8A and FIG. 8B, the results are compared to a control group and a H/R group. The cytoskeleton imaging showed RNA molecule HC50 and HC83 can significantly relieve cytoskeleton destruction of H9C2 cells caused by hypoxia/reoxygenation (H/R) injury in a dose-dependent manner.

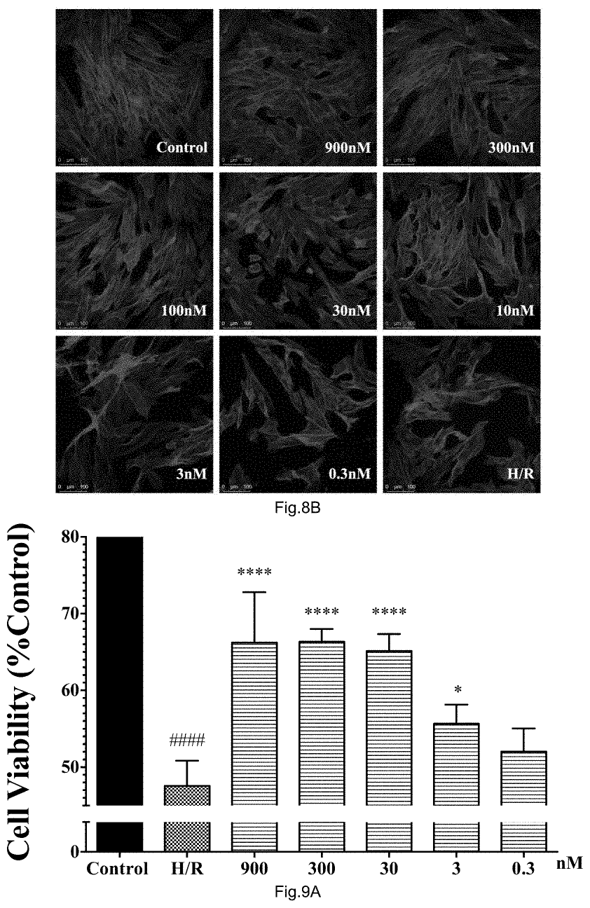

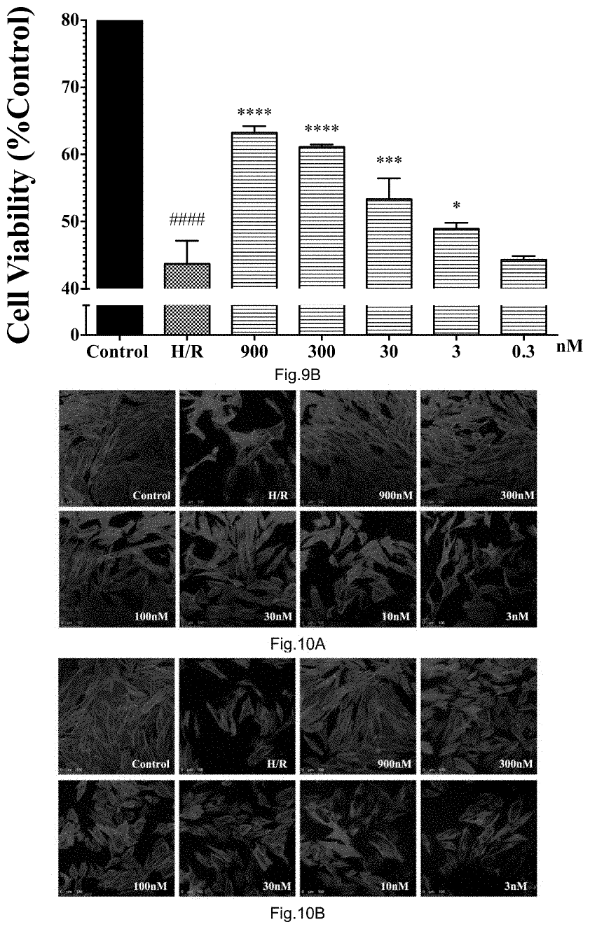

Further, the inventors determined the protective effects of cholesterol-conjugated RNA molecule HC50 and HC83 on H9C2 cells at different concentrations, i.e. 900 nM, 300 nM, 100 nM, 30 nM, 10 nM, 3 nM, and 0.3 nM. With reference to FIG. 9A and FIG. 9B, the results are compared to a control group and a H/R group. The results showed that cholesterol-conjugated RNA molecule HC50 and HC83 has a dose-dependent protective effect against hypoxia/reoxygenation (H/R) injury. The inventors also determined the protective effects of cholesterol-conjugated RNA molecule HC50 and HC83 on H9C2 cell cytoskeleton at different concentrations, i.e. 900 nM, 300 nM, 100 nM, 30 nM, 10 nM, 3 nM, and 0.3 nM. With reference to FIG. 10A and FIG. 10B, the results are compared to a control group and a H/R group. The cytoskeleton imaging showed cholesterol-conjugated RNA molecule HC50 and HC83 can significantly relieve cytoskeleton destruction of H9C2 cells caused by hypoxia/reoxygenation (H/R) injury in a dose-dependent manner.

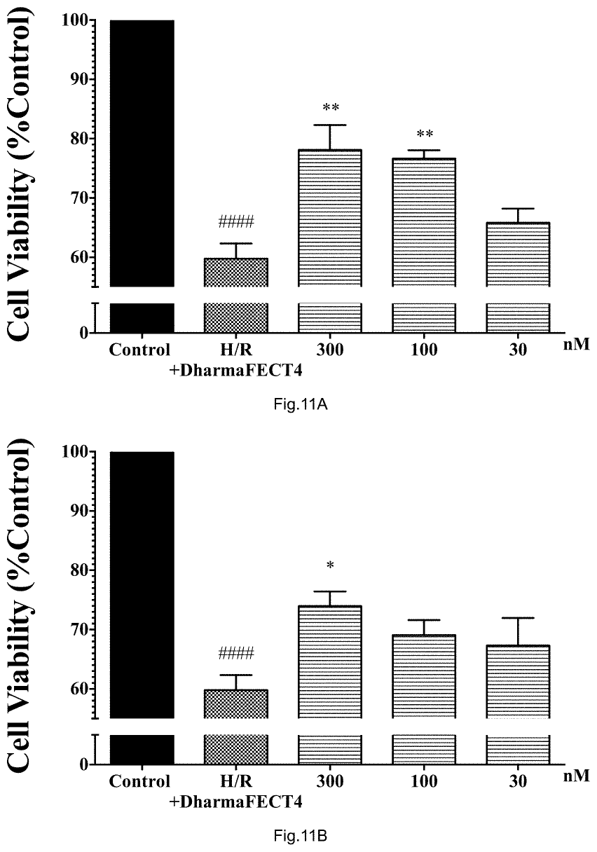

The inventors further compared the results to a control group and H/R along with DharmaFECT4 treated group (H/R+ DharmaFECT4), as shown in FIG. 11A and FIG. 11B, RNA molecule HC50 and HC83 promoted the growth and proliferation of cardiomyocytes against hypoxia/reoxygenation (H/R) injury in a dose-dependent manner.

Based on the above results, it is found that the small tRNA molecules isolated or derived from Panax ginseng C. A. Mey are highly effective on cardioprotection in vitro.

The embodiments described above are some examples of the present invention. For ordinary technicians in this field, several deformations and improvements can be made on the premise of not separating from the creative idea of the present invention, which belong to the protection scope of the present invention.

### Numbered Embodiments

The implementation is further described with reference to the following numbered embodiments:

1. A method of preventing or treating a subject suffering from heart disease comprising administering a transfer RNA molecule, a fragment derived from the transfer RNA molecule or a functional variant or homolog thereof, wherein the transfer RNA molecule is isolated from or derived from a plant of a genus Panax.

2. The method of embodiment 1, wherein the plant of the genus Panax is Panax ginseng C. A. Mey, Panax notoginseng (Burkill) F. H. Chen or Panax quinquefolius Linn.

3. The method of embodiment 1, wherein the transfer RNA molecule is a nucleic acid sequence selected from any one of SEQ ID NO: 465 to SEQ ID NO: 522.

4. The method of embodiment 1, wherein the fragment derived from the transfer RNA molecule is a double-stranded RNA molecule comprising a sense sequence selected from any one of SEQ ID NO: 1 to SEQ ID NO: 232 or a functional variant or homolog thereof, and a complementary antisense sequence.

5. The method of embodiment 1, wherein the transfer RNA molecule, the fragment derived from the transfer RNA molecule or the functional variant or homolog thereof comprises a 2 mer of 3′ overhang.

6. The method of embodiment 1, wherein the transfer RNA molecule, the fragment derived from the transfer RNA molecule or the functional variant or homolog thereof comprises a 3′ cholesterol conjugation.

7. The method of embodiment 1, wherein the transfer RNA molecule, the fragment derived from the transfer RNA molecule or the functional variant or homolog thereof comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

8. The method of embodiment 1, wherein the heart disease is selected from one or more of angina pectoris, myocardial infarction, myocardial ischemic injury, coronary heart disease, cardiac hypertrophy, and myocardial fibrosis.

9. A pharmaceutical composition for preventing or treating heart disease, wherein the pharmaceutical composition comprises an effective amount of a transfer RNA molecule, a fragment derived from the transfer RNA molecule or a functional variant or homolog thereof and a pharmaceutically tolerable vector, virus or excipient, wherein the transfer RNA molecule is isolated or derived from a plant of a genus Panax.

10. The pharmaceutical composition of embodiment 9, wherein the plant of the genus Panax is Panax ginseng C. A. Mey, Panax notoginseng (Burkill) F. H. Chen or Panax quinquefolius Linn.

11. The pharmaceutical composition of embodiment 9, wherein the transfer RNA molecule is a nucleic acid sequence selected from any one of SEQ ID NO: 465 to SEQ ID NO: 522.

12. The pharmaceutical composition of embodiment 9, wherein the fragment derived from the transfer RNA molecule is a double-stranded RNA molecule comprising a sense sequence selected from any one of SEQ ID NO: 1 to SEQ ID NO: 232 or a functional variant or homolog thereof, and a complementary antisense sequence.

13. The pharmaceutical composition of embodiment 9, wherein the transfer RNA molecule, the fragment derived from the transfer RNA molecule or the functional variant or homolog thereof comprises a 2 mer of 3′ overhang.

14. The pharmaceutical composition of embodiment 9, wherein the transfer RNA molecule, the fragment derived from the transfer RNA molecule or the functional variant or homolog thereof comprises a 3′ cholesterol conjugation.

15. The pharmaceutical composition of embodiment 9, wherein the transfer RNA molecule, the fragment derived from the transfer RNA molecule or the functional variant or homolog thereof comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

16. The pharmaceutical composition of embodiment 9, wherein the heart disease is selected from one or more of angina pectoris, myocardial infarction, myocardial ischemic injury, coronary heart disease, cardiac hypertrophy, and myocardial fibrosis.

17. A recombinant vector comprising a double-stranded RNA molecule, wherein the double-stranded RNA molecule comprises a sense sequence selected from any one of SEQ ID NO: 1 to SEQ ID NO: 232 or a functional variant or homolog thereof, and a complementary antisense sequence.

18. The recombinant vector of embodiment 17, wherein the double-stranded RNA molecule comprises a 2 mer of 3′ overhang.

19. The recombinant vector of embodiment 17, wherein the double-stranded RNA molecule comprises a 3′ cholesterol conjugation.

20. The recombinant vector of embodiment 17, wherein the double-stranded RNA molecule comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methyl inosine, pseudouridine, dihydrouridine, or 2-methylthio-N⁶-methyladenosine.

What is claimed is:

1. A method of preventing or treating a subject suffering from heart disease comprising administering a transfer RNA molecule, a fragment derived from the transfer RNA molecule or a functional variant or homolog thereof, wherein the transfer RNA molecule is isolated from or derived from a plant of a genus Panax.

2. The method of claim 1, wherein the plant of the genus Panax is Panax ginseng C. A. Mey, Panax notoginseng (Burkill) F. H. Chen or Panax quinquefolius Linn.

3. The method of claim 1, wherein the transfer RNA molecule is a nucleic acid sequence selected from any one of SEQ ID NO: 465 to SEQ ID NO: 522.

4. The method of claim 1, wherein the fragment derived from the transfer RNA molecule is a double-stranded RNA molecule comprising a sense sequence selected from any one of SEQ ID NO: 1 to SEQ ID NO: 232 or a functional variant or homolog thereof, and a complementary antisense sequence.

5. The method of claim 1, wherein the transfer RNA molecule, the fragment derived from the transfer RNA molecule or the functional variant or homolog thereof comprises a 2 mer of 3′ overhang.

6. The method of claim 1, wherein the transfer RNA molecule, the fragment derived from the transfer RNA molecule or the functional variant or homolog thereof comprises a 3′ cholesterol conjugation.

7. The method of claim 1, wherein the transfer RNA molecule, the fragment derived from the transfer RNA molecule or the functional variant or homolog thereof comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.

8. The method of claim 1, wherein the heart disease is selected from one or more of angina pectoris, myocardial infarction, myocardial ischemic injury, coronary heart disease, cardiac hypertrophy, and myocardial fibrosis.

9. A pharmaceutical composition for preventing or treating heart disease, wherein the pharmaceutical composition comprises an effective amount of a transfer RNA molecule, a fragment derived from the transfer RNA molecule or a functional variant or homolog thereof and a pharmaceutically tolerable vector, virus or excipient, wherein the transfer RNA molecule is isolated or derived from a plant of a genus Panax.

10. The pharmaceutical composition of claim 9, wherein the plant of the genus Panax is Panax ginseng C. A. Mey, Panax notoginseng (Burkill) F. H. Chen or Panax quinquefolius Linn.

11. The pharmaceutical composition of claim 9, wherein the transfer RNA molecule is a nucleic acid sequence selected from any one of SEQ ID NO: 465 to SEQ ID NO: 522.

12. The pharmaceutical composition of claim 9, wherein the fragment derived from the transfer RNA molecule is a double-stranded RNA molecule comprising a sense sequence selected from any one of SEQ ID NO: 1 to SEQ ID NO: 232 or a functional variant or homolog thereof, and a complementary antisense sequence.

13. The pharmaceutical composition of claim 9, wherein the transfer RNA molecule, the fragment derived from the transfer RNA molecule or the functional variant or homolog thereof comprises a 2 mer of 3′ overhang.

14. The pharmaceutical composition of claim 9, wherein the transfer RNA molecule, the fragment derived from the transfer RNA molecule or the functional variant or homolog thereof comprises a 3′ cholesterol conjugation.

15. The pharmaceutical composition of claim 9, wherein the transfer RNA molecule, the fragment derived from the transfer RNA molecule or the functional variant or homolog thereof comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methyl inosine, pseudouridine, dihydrouridine, or 2-methylthio-N⁶-methyladenosine.

16. The pharmaceutical composition of claim 9, wherein the heart disease is selected from one or more of angina pectoris, myocardial infarction, myocardial ischemic injury, coronary heart disease, cardiac hypertrophy, and myocardial fibrosis.

17. A recombinant vector comprising a double-stranded RNA molecule, wherein the double-stranded RNA molecule comprises a sense sequence selected from any one of SEQ ID NO: 1 to SEQ ID NO: 232 or a functional variant or homolog thereof, and a complementary antisense sequence.

18. The recombinant vector of claim 17, wherein the double-stranded RNA molecule comprises a 2 mer of 3′ overhang.

19. The recombinant vector of claim 17, wherein the double-stranded RNA molecule comprises a 3′ cholesterol conjugation.

20. The recombinant vector of claim 17, wherein the double-stranded RNA molecule comprises at least one modified nucleoside selected from inosine, 1-methyladenosine, 2-methyladenosine, N⁶-methyladenosine, N⁶-isopentenyladenosine, 2′-O-methyladenosine, N⁶-acetyladenosine, 1-methylinosine, pseudouridine, dihydrouridine, or 2-methylthio-N⁶-methyladenosine.
